(19) United States
(12) Patent Application Publication
(10) Pub. No.: US 2019/0174760 A1
(43) Pub. Date: Jun. 13, 2019

(54) N-ALKYLSULFONYL CONDENSED HETEROCYCLIC COMPOUND OR SALT THEREOF, INSECTICIDE COMPRISING THE COMPOUND, AND METHOD FOR USING THE INSECTICIDE

(71) Applicant: Nihon Nohyaku Co., Ltd., Tokyo (JP)

(72) Inventors: Yutaka Abe, Osaka (JP); Chiaki Yamauchi, Osaka (JP); Yuhji Sakurai, Osaka (JP); Hirokazu Fujihara, Osaka (JP); Soichiro Matsuo, Osaka (JP); Ikki Yonemura, Osaka (JP); Akiyuki Suwa, Osaka (JP); Shunpei Fujie, Osaka (JP); Ryosuke Tanaka, Osaka (JP)

(21) Appl. No.: 16/311,139

(22) PCT Filed: Jun. 30, 2017
(86) PCT No.: PCT/JP2017/024158

§ 371 Date: Dec. 18, 2018

(30) Foreign Application Priority Data
Jul. 1, 2016 (JP) 2016-131280
Sep. 15, 2016 (JP) 2016-180927

(51) Int. Cl. A01N 43/90; C07D 487/04; C07D 471/04; C07D 413/04

(52) U.S. Cl. CPC A01N 43/90; C07D 413/04; C07D 471/04; C07D 487/04

(57) ABSTRACT

In crop production in the fields of agriculture, horticulture and the like, the damage caused by insect pests etc. is still immense, and insect pests resistant to existing insecticides have emerged. Under such circumstances, the development of novel agricultural and horticultural insecticides is desired. The present invention provides a condensed heterocyclic compound represented by the general formula (1):

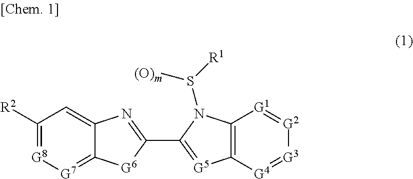

{wherein R¹ represents a (C₁-C₆) alkyl group, R² represents a halo (C₁-C₆) alkyl group, G¹, G², G³, G⁴, G⁵, G⁶, G⁷ and G⁸ each represent CH or N, and m represents an integer of 0 to 2} or a salt thereof; an agricultural and horticultural insecticide comprising the compound or the salt as an active ingredient; and a method for using the insecticide.

### TECHNICAL FIELD

The present invention relates to an insecticide comprising an N-alkylsulfonyl condensed heterocyclic compound or a salt thereof as an active ingredient, and a method for using the insecticide.

### BACKGROUND ART

Various compounds have been examined for their potential as agricultural and horticultural insecticides, and among them, certain kinds of condensed heterocyclic compounds have been reported to be useful as insecticides (for example, see Patent Literature 1 to 8). In particular, Patent Literature 8 discloses a compound having two condensed heterocycles bound to each other. However, none of the compounds disclosed in the literature have an N-alkylsulfonyl condensed heterocycle as one of the condensed heterocycles.

### CITATION LIST

### Patent Literature

Patent Literature 1: JP-A 2009-280574

Patent Literature 2: JP-A 2010-275301

Patent Literature 3: JP-A 2011-79774

Patent Literature 4: JP-A 2012-131780

Patent Literature 5: WO 2012/086848

Patent Literature 6: WO 2013/018928

Patent literature 7: WO 2014/157600

Patent literature 8: WO 2016/091731

### SUMMARY OF INVENTION

### Technical Problem

In crop production in the fields of agriculture, horticulture and the like, the damage caused by insect pests etc. is still immense, and insect pests resistant to existing insecticides have emerged. Under such circumstances, the development of novel insecticides and acaricides is desired.

### Solution to Problem

The present inventors conducted extensive research to develop a novel insecticide, particularly an agricultural and horticultural insecticide. As a result, the present inventors found that a compound represented by the general formula (1) of the present invention which has an N-alkylsulfonyl condensed heterocycle or a salt of the compound is highly effective as an insecticide. Based on this finding, the present inventors completed the present invention.

That is, the present invention includes the following.

[1] A compound represented by the general formula (1):

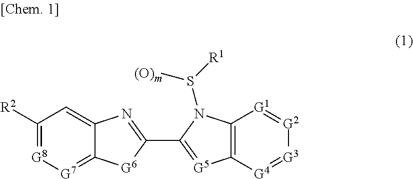

{wherein

R¹ represents

(a1) a (C₁-C₆) alkyl group;
(a2) a (C₃-C₆) cycloalkyl group;
(a3) a (C₂-C₆) alkenyl group; or
(a4) a (C₂-C₆) alkynyl group,

R² represents

(b1) a halogen atom;
(b2) a cyano group;
(b3) a nitro group;
(b4) a (C₁-C₆) alkyl group;
(b5) a (C₁-C₆) alkoxy group;
(b6) a (C₂-C₆) alkenyloxy group;
(b7) a (C₂-C₆) alkynyloxy group;
(b8) a halo (C₁-C₆) alkyl group;
(b9) a halo (C₁-C₆) alkoxy group;
(b10) a halo (C₂-C₆) alkenyloxy group;
(b11) a halo (C₂-C₆) alkynyloxy group;
(b12) a (C₁-C₆) alkylthio group;
(b13) a (C₁-C₆) alkylsulfinyl group;
(b14) a (C₁-C₆) alkylsulfonyl group;
(b15) a halo (C₁-C₆) alkylthio group;
(b16) a halo (C₁-C₆) alkylsulfinyl group; or
(b17) a halo (C₁-C₆) alkylsulfonyl group,

G¹, G², G³ and G⁴ independently represent C—R³ or N

(wherein

each R³ independently represents

(c1) a hydrogen atom;
(c2) a halogen atom;
(c3) a hydroxyl group;
(c4) a formyl group;
(c5) a cyano group;
(c6) a (C₁-C₆) alkyl group;
(c7) a (C₃-C₆) cycloalkyl group;
(c8) a (C₁-C₆) alkoxy group;
(c9) a halo (C₁-C₆) alkyl group;
(c10) a halo (C₃-C₆) cycloalkyl group;
(c11) a halo (C₁-C₆) alkoxy group;
(c12) a (C₁-C₆) alkylthio group;
(c13) a (C₁-C₆) alkylsulfinyl group;
(c14) a (C₁-C₆) alkylsulfonyl group;
(c15) a halo (C₁-C₆) alkylthio group;
(c16) a halo (C₁-C₆) alkylsulfinyl group;
(c17) a halo (C₁-C₆) alkylsulfonyl group;
(c18) a (C₃-C₆) cycloalkyl (C₁-C₆) alkoxy group;
(c19) an R⁹(R¹⁰)N group (wherein R⁹ and R¹⁰ independently represent a hydrogen atom; a (C₁-C₆) alkyl group; a (C₃-C₆) cycloalkyl (C₁-C₆) alkyl group; a halo (C₁-C₆) alkyl group; a (C₁-C₆) alkylcarbonyl group; a (C₁-C₆) alkoxycarbonyl group; or a phenylcarbonyl group);
(c20) a mono (C₁-C₆) alkylaminocarbonyl NH group;
(c21) a mono (C₁-C₆) alkylaminothiocarbonyl NH group;
(c22) an R¹⁰ON═C(R⁹) group (wherein R⁹ and R¹⁰ are as defined above);
(c23) a carboxyl group;
(c24) a (C₁-C₆) alkoxycarbonyl group;
(c25) an R⁹(R¹⁰)N carbonyl group (wherein R⁹ and R¹⁰ are as defined above);
(c26) an aryl group;
(c27) an aryl group having, on the ring, 1 to 5 substituting groups independently selected from the group consisting of a halogen atom, a (C₁-C₆) alkyl group, a halo (C₁-C₆) alkyl group, a (C₁-C₆) alkoxy group, a halo (C₁-C₆) alkoxy group, a (C₁-C₆) alkylthio group, a halo (C₁-C₆) alkylthio group, a (C₁-C₆) alkylsulfinyl group, a halo (C₁-C₆) alkylsulfinyl group, a (C₁-C₆) alkylsulfonyl group and a halo (C₁-C₆) alkylsulfonyl group;
(c28) an aryl (C₁-C₆) alkyl group;
(c29) an aryl (C₁-C₆) alkyl group having, on the ring, 1 to 5 substituting groups independently selected from the group consisting of a halogen atom, a (C₁-C₆) alkyl group, a halo (C₁-C₆) alkyl group, a (C₁-C₆) alkoxy group, a halo (C₁-C₆) alkoxy group, a (C₁-C₆) alkylthio group, a halo (C₁-C₆) alkylthio group, a (C₁-C₆) alkylsulfinyl group, a halo (C₁-C₆) alkylsulfinyl group, a (C₁-C₆) alkylsulfonyl group and a halo (C₁-C₆) alkylsulfonyl group;
(c30) an aryl (C₁-C₆) alkoxy group;
(c31) an aryl (C₁-C₆) alkoxy group having, on the ring, 1 to 5 substituting groups independently selected from the group consisting of a halogen atom, a (C₁-C₆) alkyl group, a halo (C₁-C₆) alkyl group, a (C₁-C₆) alkoxy group, a halo (C₁-C₆) alkoxy group, a (C₁-C₆) alkylthio group, a halo (C₁-C₆) alkylthio group, a (C₁-C₆) alkylsulfinyl group, a halo (C₁-C₆) alkylsulfinyl group, a (C₁-C₆) alkylsulfonyl group and a halo (C₁-C₆) alkylsulfonyl group;
(c32) an aryl (C₁-C₆) alkyl NH group;
(c33) an aryl (C₁-C₆) alkyl NH group having, on the ring, 1 to 5 substituting groups independently selected from the group consisting of a halogen atom, a (C₁-C₆) alkyl group, a halo (C₁-C₆) alkyl group, a (C₁-C₆) alkoxy group, a halo (C₁-C₆) alkoxy group, a (C₁-C₆) alkylthio group, a halo (C₁-C₆) alkylthio group, a (C₁-C₆) alkylsulfinyl group, a halo (C₁-C₆) alkylsulfinyl group, a (C₁-C₆) alkylsulfonyl group and a halo (C₁-C₆) alkylsulfonyl group;
(c34) a heterocyclic group;
(c35) a heterocyclic group having, on the ring, 1 to 3 substituting groups independently selected from the group consisting of a halogen atom, a (C₁-C₆) alkyl group, a halo (C₁-C₆) alkyl group, a (C₁-C₆) alkoxy group, a halo (C₁-C₆) alkoxy group, a (C₁-C₆) alkylthio group, a halo (C₁-C₆) alkylthio group, a (C₁-C₆) alkylsulfinyl group, a halo (C₁-C₆) alkylsulfinyl group, a (C₁-C₆) alkylsulfonyl group and a halo (C₁-C₆) alkylsulfonyl group; or
(c36) a (C₁-C₆) alkoxycarbonyl NH group),

G⁵ represents C—R⁴ or N

(wherein

R⁴ represents

(d1) a hydrogen atom;
(d2) a halogen atom;
(d3) a (C₁-C₆) alkyl group;
(d4) a (C₃-C₆) cycloalkyl group;
(d5) a (C₂-C₆) alkenyl group;
(d6) a (C₂-C₆) alkynyl group;
(d7) a (C₁-C₆) alkoxy group;
(d8) a halo (C₁-C₆) alkyl group;
(d9) a halo (C₃-C₆) cycloalkyl group;
(d10) a halo (C₂-C₆) alkenyl group;
(d11) a halo (C₂-C₆) alkynyl group;
(d12) a halo (C₁-C₆) alkoxy group;
(d13) a cyano group;
(d14) an R⁹(R¹⁰)N group (wherein R⁹ and R¹⁰ are as defined above);
(d15) a (C₁-C₆) alkylthio group;
(d16) a (C₁-C₆) alkylsulfinyl group;
(d17) a (C₁-C₆) alkylsulfonyl group;
(d18) a (C₁-C₆) alkylthio group;
(d19) a formyl group;
(d20) a (C₁-C₆) alkoxycarbonyl group;
(d21) an R⁹(R¹⁰)N carbonyl group (wherein R⁹ and R¹⁰ are as defined above); or
(d22) an R¹⁰ON═C(R⁹) group (wherein R⁹ and R¹⁰ are as defined above)),

G⁶ represents N—R⁵; O; or S

(wherein

R⁵ represents

(e1) a hydrogen atom;
(e2) a (C₁-C₆) alkyl group;
(e3) a (C₃-C₆) cycloalkyl group;
(e4) a (C₂-C₆) alkenyl group; or
(e5) a (C₂-C₆) alkynyl group),
G⁷ and G⁸ independently represent C—H or N, and
m represents an integer of 0 to 2}, or
   a salt thereof.
   [2] The compound or the salt according to the above [1], wherein

R¹ is (a1) a (C₁-C₆) alkyl group,

R² is

(b8) a halo (C₁-C₆) alkyl group;
(b15) a halo (C₁-C₆) alkylthio group; or
(b17) a halo (C₁-C₆) alkylsulfonyl group,

each R³ is independently

(c1) a hydrogen atom;
(c2) a halogen atom;
(c4) a formyl group;
(c5) a cyano group;
(c6) a (C₁-C₆) alkyl group;
(c7) a (C₃-C₆) cycloalkyl group;
(c8) a (C₁-C₆) alkoxy group;
(c9) a halo (C₁-C₆) alkyl group;
(c11) a halo (C₁-C₆) alkoxy group;
(c12) a (C₁-C₆) alkylthio group;
(c13) a (C₁-C₆) alkylsulfinyl group;
(c14) a (C₁-C₆) alkylsulfonyl group;
(c15) a halo (C₁-C₆) alkylthio group;
(c19) an R⁹(R¹⁰)N group (wherein R⁹ and R¹⁰ independently represent a hydrogen atom; a (C₁-C₆) alkyl group; a (C₃-C₆) cycloalkyl (C₁-C₆) alkyl group; a halo (C₁-C₆) alkyl group; a (C₁-C₆) alkylcarbonyl group; a (C₁-C₆) alkoxycarbonyl group; or a phenylcarbonyl group);
(c22) an R¹⁰ON═C(R⁹) group (wherein R⁹ and R¹⁰ are as defined above);
(c24) a (C₁-C₆) alkoxycarbonyl group;
(c25) an R⁹(R¹⁰)N carbonyl group (wherein R⁹ and R¹⁰ are as defined above);
(c27) an aryl group having, on the ring, 1 to 5 substituting groups independently selected from the group consisting of a halogen atom, a (C₁-C₆) alkyl group, a halo (C₁-C₆) alkyl group, a (C₁-C₆) alkoxy group, a halo (C₁-C₆) alkoxy group, a (C₁-C₆) alkylthio group, a halo (C₁-C₆) alkylthio group, a (C₁-C₆) alkylsulfinyl group, a halo (C₁-C₆) alkylsulfinyl group, a (C₁-C₆) alkylsulfonyl group and a halo (C₁-C₆) alkylsulfonyl group; or
(c36) a (C₁-C₆) alkoxycarbonyl NH group,

R⁴ is

(d1) a hydrogen atom;
(d2) a halogen atom;
(d3) a (C₁-C₆) alkyl group;
(d4) a (C₃-C₆) cycloalkyl group;
(d7) a (C₁-C₆) alkoxy group;
(d12) a halo (C₁-C₆) alkoxy group;
(d13) a cyano group;
(d14) an R⁹(R¹⁰)N group (wherein R⁹ and R¹⁰ independently represent a hydrogen atom; a (C₁-C₆) alkyl group; a (C₃-C₆) cycloalkyl (C₁-C₆) alkyl group; a halo (C₁-C₆) alkyl group; a (C₁-C₆) alkylcarbonyl group; a (C₁-C₆) alkoxycarbonyl group; or a phenylcarbonyl group);
(d15) a (C₁-C₆) alkylthio group;
(d16) a (C₁-C₆) alkylsulfinyl group;
(d17) a (C₁-C₆) alkylsulfonyl group;
(d18) a (C₁-C₆) alkylthio group;
(d19) a formyl group;
(d20) a (C₁-C₆) alkoxycarbonyl group;
(d21) an R⁹(R¹⁰)N carbonyl group (wherein R⁹ and R²⁰ are as defined above); or
(d22) an R¹⁰ON═C(R⁹) group (wherein R⁹ and R¹⁰ are as defined above),

G⁶ is N—R⁵ or O, and

R⁵ is (e2) a (C₁-C₆) alkyl group.

[3] The compound or the salt according to the above [1] or [2], wherein

R² is

(b8) a halo (C₁-C₆) alkyl group or
(b15) a halo (C₁-C₆) alkylthio group, each R³ is independently
(c1) a hydrogen atom;
(c2) a halogen atom;
(c8) a (C₁-C₆) alkoxy group;
(c9) a halo (C₁-C₆) alkyl group; or
(c11) a halo (C₁-C₆) alkoxy group, and

R⁴ is

(d1) a hydrogen atom or
(d2) a halogen atom.
[4] An insecticide comprising the compound or the salt according to any of the above [1] to [3] as an active ingredient.
[5] An agricultural and horticultural insecticide comprising the compound or the salt according to any of the above [1] to [3] as an active ingredient.
[6] An animal ectoparasite control agent comprising the compound or the salt according to any of the above [1] to [3] as an active ingredient.
[7] A method for using an insecticide, comprising treating plants or soil with an effective amount of the insecticide according to the above [4].
[8] Use of the compound or the salt according to any of the above [1] to [3] as an insecticide.
[9] A condensed heterocyclic compound represented by the general formula (1):

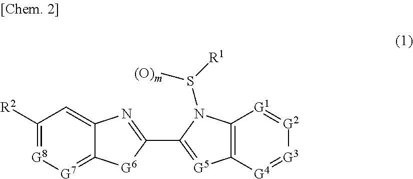

{wherein

R₁ represents

(a1) a (C₁-C₆) alkyl group;
(a2) a (C₃-C₆) cycloalkyl group;
(a3) a (C₂-C₆) alkenyl group; or
(a4) a (C₂-C₆) alkynyl group,

R² represents

(b1) a halogen atom;
(b2) a cyano group;
(b3) a nitro group;
(b4) a (C₁-C₆) alkyl group;
(b5) a (C₁-C₆) alkoxy group;
(b6) a (C₂-C₆) alkenyloxy group;
(b7) a (C₂-C₆) alkynyloxy group;
(b8) a halo (C₁-C₆) alkyl group;
(b9) a halo (C₁-C₆) alkoxy group;
(b10) a halo (C₂-C₆) alkenyloxy group;
(b11) a halo (C₂-C₆) alkynyloxy group;
(b12) a (C₁-C₆) alkylthio group;
(b13) a (C₁-C₆) alkylsulfinyl group;
(b14) a (C₁-C₆) alkylsulfonyl group;
(b15) a halo (C₁-C₆) alkylthio group;
(b16) a halo (C₁-C₆) alkylsulfinyl group; or
(b17) a halo (C₁-C₆) alkylsulfonyl group,

G¹, G², G³ and G⁴ may be the same or different and each represent C—R³ or N

(wherein

R³s may be the same or different and represents

(c1) a hydrogen atom;
(c2) a halogen atom;
(c3) a hydroxyl group;
(c4) a formyl group;
(c5) a cyano group;
(c6) a (C₁-C₆) alkyl group;
(c7) a (C₃-C₆) cycloalkyl group;
(c8) a (C₁-C₆) alkoxy group;
(c9) a halo (C₁-C₆) alkyl group;
(c10) a halo (C₃-C₆) cycloalkyl group;
(c11) a halo (C₁-C₆) alkoxy group;
(c12) a (C₁-C₆) alkylthio group;
(c13) a (C₁-C₆) alkylsulfinyl group;
(c14) a (C₁-C₆) alkylsulfonyl group;
(c15) a halo (C₁-C₆) alkylthio group;
(c16) a halo (C₁-C₆) alkylsulfinyl group;
(c17) a halo (C₁-C₆) alkylsulfonyl group;
(c18) a (C₃-C₆) cycloalkyl (C₁-C₆) alkoxy group;
(c19) an R⁹(R¹⁰)N group (wherein R⁹ and R¹⁰ may be the same or different and each represent a hydrogen atom; a (C₁-C₆) alkyl group; a (C₃-C₆) cycloalkyl (C₁-C₆) alkyl group; a halo (C₁-C₆) alkyl group; a (C₁-C₆) alkylcarbonyl group; a (C₁-C₆) alkoxycarbonyl group; or a phenylcarbonyl group);
(c20) a mono (C₁-C₆) alkylaminocarbonyl NH group;
(c21) a mono (C₁-C₆) alkylaminothiocarbonyl NH group;
(c22) a C(R⁹)═NOR¹⁰ group (wherein R⁹ and R¹⁰ are as defined above);
(c23) a carboxyl group;
(c24) a (C₁-C₆) alkoxycarbonyl group;
(c25) an R⁹(R¹⁰)N carbonyl group (wherein R⁹ and R¹⁰ are as defined above);
(c26) an aryl group;
(c27) an aryl group having, on the ring, 1 to 5 substituting groups which may be the same or different and are selected from the group consisting of a halogen atom, a
(C₁-C₆) alkyl group, a halo (C₁-C₆) alkyl group, a (C₁-C₆) alkoxy group, a halo (C₁-C₆) alkoxy group, a (C₁-C₆) alkylthio group, a halo (C₁-C₆) alkylthio group, a (C₁-C₆) alkylsulfinyl group, a halo (C₁-C₆) alkylsulfinyl group, a (C₁-C₆) alkylsulfonyl group and a halo (C₁-C₆) alkylsulfonyl group;
(c28) an aryl (C₁-C₆) alkyl group;
(c29) an aryl (C₁-C₆) alkyl group having, on the ring, 1 to 5 substituting groups which may be the same or different and are selected from the group consisting of a halogen atom, a (C₁-C₆) alkyl group, a halo (C₁-C₆) alkyl group, a (C₁-C₆) alkoxy group, a halo (C₁-C₆) alkoxy group, a (C₁-C₆) alkylthio group, a halo (C₁-C₆) alkylthio group, a (C₁-C₆) alkylsulfinyl group, a halo (C₁-C₆) alkylsulfinyl group, a (C₁-C₆) alkylsulfonyl group and a halo (C₁-C₆) alkylsulfonyl group;
(c30) an aryl (C₁-C₆) alkoxy group;
(c31) an aryl (C₁-C₆) alkoxy group having, on the ring, 1 to 5 substituting groups which may be the same or different and are selected from the group consisting of a halogen atom, a (C₁-C₆) alkyl group, a halo (C₁-C₆) alkyl group, a (C₁-C₆) alkoxy group, a halo (C₁-C₆) alkoxy group, a (C₁-C₆) alkylthio group, a halo (C₁-C₆) alkylthio group, a (C₁-C₆) alkylsulfinyl group, a halo (C₁-C₆) alkylsulfinyl group, a (C₁-C₆) alkylsulfonyl group and a halo (C₁-C₆) alkylsulfonyl group;
(c32) an aryl (C₁-C₆) alkyl NH group;
(c33) an aryl (C₁-C₆) alkyl NH group having, on the ring, 1 to 5 substituting groups which may be the same or different and are selected from the group consisting of a halogen atom, a (C₁-C₆) alkyl group, a halo (C₁-C₆) alkyl group, a (C₁-C₆) alkoxy group, a halo (C₁-C₆) alkoxy group, a (C₁-C₆) alkylthio group, a halo (C₁-C₆) alkylthio group, a (C₁-C₆) alkylsulfinyl group, a halo (C₁-C₆) alkylsulfinyl group, a (C₁-C₆) alkylsulfonyl group and a halo (C₁-C₆) alkylsulfonyl group;
(c34) a heterocyclic group; or
(c35) a heterocyclic group having, on the ring, 1 to 3 substituting groups which may be the same or different and are selected from the group consisting of a halogen atom, a (C₁-C₆) alkyl group, a halo (C₁-C₆) alkyl group, a (C₁-C₆) alkoxy group, a halo (C₁-C₆) alkoxy group, a (C₁-C₆) alkylthio group, a halo (C₁-C₆) alkylthio group, a (C₁-C₆) alkylsulfinyl group, a halo (C₁-C₆) alkylsulfinyl group, a (C₁-C₆) alkylsulfonyl group and a halo (C₁-C₆) alkylsulfonyl group),

G⁵ represents C—R⁴ or N

(wherein

R⁴ represents

(d1) a hydrogen atom;
(d2) a halogen atom;
(d3) a (C₁-C₆) alkyl group;
(d4) a (C₃-C₆) cycloalkyl group;
(d5) a (C₂-C₆) alkenyl group;
(d6) a (C₂-C₆) alkynyl group;
(d7) a (C₁-C₆) alkoxy group;
(d8) a halo (C₁-C₆) alkyl group;
(d9) a halo (C₃-C₆) cycloalkyl group;
(d10) a halo (C₂-C₆) alkenyl group;
(d11) a halo (C₂-C₆) alkynyl group; or
(d12) a halo (C₁-C₆) alkoxy group),

G⁶ represents N—R⁵, O or S

(wherein

R⁵ represents

(e1) a hydrogen atom;
(e2) a (C₁-C₆) alkyl group;
(e3) a (C₃-C₆) cycloalkyl group;
(e4) a (C₂-C₆) alkenyl group; or
(e5) a (C₂-C₆) alkynyl group),

G⁷ and G⁸ may be the same or different and each represent C—H or N, and

m represents an integer of 0 to 2}, or

a salt thereof.
[10] The condensed heterocyclic compound or the salt according to the above [9], wherein

R₁ represents (a1) a (C₁-C₆) alkyl group,

R² is

(b8) a halo (C₁-C₆) alkyl group;
(b15) a halo (C₁-C₆) alkylthio group; or
(b17) a halo (C₁-C₆) alkylsulfonyl group,

G¹, G², G³ and G⁴ each represent C—R³

(wherein

R³s may be the same or different and represents

(c1) a hydrogen atom;
(c2) a halogen atom;
(c4) a formyl group;
(c5) a cyano group;
(c6) a (C₁-C₆) alkyl group;
(c7) a (C₃-C₆) cycloalkyl group;
(c8) a (C₁-C₆) alkoxy group;
(c9) a halo (C₁-C₆) alkyl group;
(c12) a (C₁-C₆) alkylthio group;
(c15) a halo (C₁-C₆) alkylthio group;
(c19) an R⁹(R¹⁰)N group (wherein R⁹ and R²⁹ may be the same or different and each represent a hydrogen atom; a (C₁-C₆) alkyl group; a (C₃-C₆) cycloalkyl (C₁-C₆) alkyl group; a halo (C₁-C₆) alkyl group; a (C₁-C₆) alkylcarbonyl group; a (C₁-C₆) alkoxycarbonyl group; or a phenylcarbonyl group);
(c22) a C(R⁹)═NOR¹⁰ group (wherein R⁹ and R²⁹ are as defined above);
(c24) a (C₁-C₆) alkoxycarbonyl group; or
(c25) an R⁹(R¹⁰)N carbonyl group (wherein R⁹ and R²⁰ are as defined above)),

G⁵ represents C—R⁴ (wherein R⁴ represents (d1) a hydrogen atom), and

G⁶ represents N—R⁵, O or S (wherein R⁵ represents (e2) a (C₁-C₆) alkyl group).

[11] The condensed heterocyclic compound or the salt according to the above [9] or [10], wherein

R² is

(b8) a halo (C₁-C₆) alkyl group or
(b15) a halo (C₁-C₆) alkylthio group,

G², G², G³ and G⁴ each represent C—R³

(wherein

R³s may be the same or different and represents

(c1) a hydrogen atom;
(c2) a halogen atom; or
(c9) a halo (C₁-C₆) alkyl group), and

G⁶ represents N—R⁵ or O (wherein R⁵ represents (e2) a (C₁-C₆) alkyl group).

[12] An agricultural and horticultural insecticide comprising the condensed heterocyclic compound or the salt according to any of the above [9] to [11] as an active ingredient.
[13] A method for using an agricultural and horticultural insecticide, comprising treating plants or soil with an effective amount of the agricultural and horticultural insecticide according to the above [12].
[14] Use of the condensed heterocyclic compound or the salt according to any of the above [9] to [11] as an agricultural and horticultural insecticide.

### Advantageous Effects of Invention

The compound of the present invention or a salt thereof is highly effective as an insecticide. The compound of the present invention or a salt thereof is effective not only against agricultural and horticultural pests but also against pests which live on pets such as dogs and cats and domestic animals such as cattle and sheep.

### DESCRIPTION OF EMBODIMENTS

In the definitions of the general formula (1) representing the compound of the present invention, “halo” refers to a “halogen atom” and represents a chlorine atom, a bromine atom, an iodine atom or a fluorine atom.

The “(C₁-C₆) alkyl group” refers to a straight-chain or branched-chain alkyl group of 1 to 6 carbon atoms, for example, a methyl group, an ethyl group, a n-propyl group, an isopropyl group, a n-butyl group, an isobutyl group, a sec-butyl group, a tert-butyl group, a n-pentyl group, an isopentyl group, a tert-pentyl group, a neopentyl group, a 2,3-dimethylpropyl group, a 1-ethylpropyl group, a 1-methylbutyl group, a 2-methylbutyl group, a n-hexyl group, an isohexyl group, a 2-hexyl group, a 3-hexyl group, a 2-methylpentyl group, a 3-methylpentyl group, a 1,1,2-trimethyl propyl group, a 3,3-dimethylbutyl group or the like.

The “(C₂-C₆) alkenyl group” refers to a straight-chain or branched-chain alkenyl group of 2 to 6 carbon atoms, for example, a vinyl group, an allyl group, an isopropenyl group, a 1-butenyl group, a 2-butenyl group, a 2-methyl-2-propenyl group, a 1-methyl-2-propenyl group, a 2-methyl-1-propenyl group, a pentenyl group, a 1-hexenyl group, a 3,3-dimethyl-1-butenyl group or the like. The “(C₂-C₆) alkynyl group” refers to a straight-chain or branched-chain alkynyl group of 2 to 6 carbon atoms, for example, an ethynyl group, a 1-propynyl group, a 2-propynyl group, a 1-butynyl group, a 2-butynyl group, a 3-butynyl group, a 3-methyl-1-propynyl group, a 2-methyl-3-propynyl group, a pentynyl group, a 1-hexynyl group, a 3-methyl-1-butynyl group, a 3,3-dimethyl-1-butynyl group or the like.

The “(C₃-C₆) cycloalkyl group” refers to a cyclic alkyl group of 3 to 6 carbon atoms, for example, a cyclopropyl group, a cyclobutyl group, a cyclopentyl group, a cyclohexyl group or the like. The “(C₁-C₆) alkoxy group” refers to a straight-chain or branched-chain alkoxy group of 1 to 6 carbon atoms, for example, a methoxy group, an ethoxy group, a n-propoxy group, an isopropoxy group, a n-butoxy group, a sec-butoxy group, a tert-butoxy group, a n-pentyloxy group, an isopentyloxy group, a tert-pentyloxy group, a neopentyloxy group, a 2,3-dimethylpropyloxy group, a 1-ethylpropyloxy group, a 1-methylbutyloxy group, a n-hexyloxy group, an isohexyloxy group, a 1,1,2-trimethylpropyloxy group or the like.

The “(C₂-C₆) alkenyloxy group” refers to a straight-chain or branched-chain alkenyloxy group of 2 to 6 carbon atoms, for example, a propenyloxy group, a butenyloxy group, a pentenyloxy group, a hexenyloxy group or the like. The “(C₂-C₆) alkynyloxy group” refers to a straight-chain or branched-chain alkynyloxy group of 2 to 6 carbon atoms, for example, a propynyloxy group, a butynyloxy group, a pentynyloxy group, a hexynyloxy group or the like.

The “(C₁-C₆) alkylthio group” refers to a straight-chain or branched-chain alkylthio group of 1 to 6 carbon atoms, for example, a methylthio group, an ethylthio group, a n-propylthio group, an isopropylthio group, a n-butylthio group, a sec-butylthio group, a tert-butylthio group, a n-pentylthio group, an isopentylthio group, a tert-pentylthio group, a neopentylthio group, a 2,3-dimethylpropylthio group, a 1-ethylpropylthio group, a 1-methylbutylthio group, a n-hexylthio group, an isohexylthio group, a 1,1,2-trimethylpropylthio group or the like.

The “(C₁-C₆) alkylsulfinyl group” refers to a straight-chain or branched-chain alkylsulfinyl group of 1 to 6 carbon atoms, for example, a methylsulfinyl group, an ethylsulfinyl group, a n-propylsulfinyl group, an isopropylsulfinyl group, a n-butylsulfinyl group, a sec-butylsulfinyl group, a tert-butylsulfinyl group, a n-pentylsulfinyl group, an isopentylsulfinyl group, a tert-pentylsulfinyl group, a neopentylsulfinyl group, a 2,3-dimethylpropylsulfinyl group, a 1-ethylpropylsulfinyl group, a 1-methylbutylsulfinyl group, a n-hexylsulfinyl group, an isohexylsulfinyl group, a 1,1,2-trimethylpropylsulfinyl group or the like.

The “(C₁-C₆) alkylsulfonyl group” refers to a straight-chain or branched-chain alkylsulfonyl group of 1 to 6 carbon atoms, for example, a methylsulfonyl group, an ethylsulfonyl group, a n-propylsulfonyl group, an isopropylsulfonyl group, a n-butylsulfonyl group, a sec-butylsulfonyl group, a tert-butylsulfonyl group, a n-pentylsulfonyl group, an isopentylsulfonyl group, a tert-pentylsulfonyl group, a neopentylsulfonyl group, a 2,3-dimethylpropylsulfonyl group, a 1-ethylpropylsulfonyl group, a 1-methylbutylsulfonyl group, a n-hexylsulfonyl group, an isohexylsulfonyl group, a 1,1,2-trimethylpropylsulfonyl group or the like.

The above-mentioned “(C₁-C₆) alkyl group”, “(C₂-C₆) alkenyl group”, “(C₂-C₆) alkynyl group”, “(C₁-C₆) alkoxy group”, “(C₂-C₆) alkenyloxy group”, “(C₂-C₆) alkynyloxy group”, “(C₁-C₆) alkylthio group”, “(C₁-C₆) alkylsulfinyl group”, “(C₁-C₆) alkylsulfonyl group” and “(C₃-C₆) cycloalkyl group” may be substituted with one or more halogen atoms at a substitutable position(s), and in the case where any of the above-listed groups is substituted with two or more halogen atoms, the halogen atoms may be the same or different.

The above-mentioned “groups substituted with one or more halogen atoms” are expressed as a “halo (C₁-C₆) alkyl group”, a “halo (C₂-C₆) alkenyl group”, a “halo (C₂-C₆) alkynyl group”, a “halo (C₁-C₆) alkoxy group”, a “halo (C₂-C₆) alkenyloxy group”, a “halo (C₂-C₆) alkynyloxy group”, a “halo (C₁-C₆) alkylthio group”, a “halo (C₁-C₆) alkylsulfinyl group”, a “halo (C₁-C₆) alkylsulfonyl group” and a “halo (C₃-C₆) cycloalkyl group”.

The expressions “(C₁-C₆)”, “(C₂-C₆)”, “(C₃-C₆)”, etc. each refer to the range of the number of carbon atoms in each group. The same definition holds true for groups in which two or more of the above-mentioned groups are coupled together, and for example, the “(C₁-C₆) alkoxy (C₁-C₆) alkyl group” means that a straight-chain or branched-chain alkoxy group of 1 to 6 carbon atoms is bound to a straight-chain or branched-chain alkyl group of 1 to 6 carbon atoms.

The “aryl group” refers to an aromatic hydrocarbon group of 6 to 10 carbon atoms, for example, a phenyl group, a 1-naphthyl group, a 2-naphthyl group or the like.

The “heterocyclic group” and the “heterocyclic ring” refer to a 5- or 6-membered monocyclic aromatic heterocyclic group containing, as ring atoms, one or more carbon atoms and 1 to 4 heteroatoms selected from an oxygen atom, a sulfur atom and a nitrogen atom; or a 4- to 6-membered monocyclic non-aromatic heterocyclic group containing, as ring atoms, one or more carbon atoms and 1 to 4 heteroatoms selected from an oxygen atom, a sulfur atom and a nitrogen atom.

Examples of the “aromatic heterocyclic group” include monocyclic aromatic heterocyclic groups such as furyl, thienyl, pyridyl, pyrimidinyl, pyridazinyl, pyrazinyl, pyrrolyl, imidazolyl, pyrazolyl, triazolyl, isothiazolyl, oxazolyl, isoxazolyl, oxadiazolyl, thiadiazolyl, triazolyl, tetrazolyl and triazinyl.

Examples of the “non-aromatic heterocyclic group” include monocyclic non-aromatic heterocyclic groups such as oxetanyl, thietanyl, azetidinyl, pyrrolidinyl, piperidinyl, morpholinyl, thiomorpholinyl, piperazinyl, hexamethyleneiminyl, oxazolidinyl, thiazolidinyl, imidazolidinyl, oxazolinyl, thiazolinyl, isoxazolinyl, imidazolinyl, dioxolyl, dioxolanyl, dihydrooxadiazolyl, 2-oxo-pyrrolidin-1-yl, 2-oxo-1,3-oxazolidin-5-yl, 5-oxo-1,2,4-oxadiazolin-3-yl, 1,3-dioxolan-2-yl, 1,3-dioxan-2-yl, 1,3-dioxepan-2-yl, pyranyl, tetrahydropyranyl, thiopyranyl, tetrahydrothiopyranyl, 1-oxide tetrahydrothiopyranyl, 1,1-dioxide tetrahydrothiopyranyl, tetrahydrofuranyl, dioxanyl, pyrazolidinyl, pyrazolinyl, tetrahydropyrimidinyl, dihydrotriazolyl and tetrahydrotriazolyl.

Examples of the salt of the compound represented by the general formula (1) of the present invention include inorganic acid salts, such as hydrochlorides, sulfates, nitrates and phosphates; organic acid salts, such as acetates, fumarates, maleates, oxalates, methanesulfonates, benzenesulfonates and p-toluenesulfonates; and salts with an inorganic or organic base such as a sodium ion, a potassium ion, a calcium ion and a trimethylammonium ion.

The compound represented by the general formula (1) of the present invention and a salt thereof can have one or more chiral centers in the structural formula, and can exist as two or more kinds of optical isomers or diastereomers. All the optical isomers and mixtures of the isomers at any ratio are also included in the present invention. Further, the compound represented by the general formula (1) of the present invention and a salt thereof can exist as two kinds of geometric isomers due to a carbon-carbon double bond in the structural formula. All the geometric isomers and mixtures of the isomers at any ratio are also included in the present invention.

Preferable embodiments of the compound represented by the general formula (1) of the present invention are described below.

R¹ is preferably (a1) a (C₁-C₆) alkyl group.

R² is preferably

(b8) a halo (C₁-C₆) alkyl group;
(b15) a halo (C₁-C₆) alkylthio group; or
(b17) a halo (C₁-C₆) alkylsulfonyl group, and more preferably
(b8) a halo (C₁-C₆) alkyl group or
(b15) a halo (C₁-C₆) alkylthio group.

Each R³ is independently

(c1) a hydrogen atom;
(c2) a halogen atom;
(c4) a formyl group;
(c5) a cyano group;
(c6) a (C₁-C₆) alkyl group;
(c7) a (C₃-C₆) cycloalkyl group;
(c8) a (C₁-C₆) alkoxy group;
(c9) a halo (C₁-C₆) alkyl group;
(c11) a halo (C₁-C₆) alkoxy group;
(c12) a (C₁-C₆) alkylthio group;
(c13) a (C₁-C₆) alkylsulfinyl group;
(c14) a (C₁-C₆) alkylsulfonyl group;
(c15) a halo (C₁-C₆) alkylthio group;
(c19) an R⁹(R¹⁰)N group (wherein R⁹ and R¹⁰ independently represent a hydrogen atom; a (C₁-C₆) alkyl group; a (C₃-C₆) cycloalkyl (C₁-C₆) alkyl group; a halo (C₁-C₆) alkyl group; a (C₁-C₆) alkylcarbonyl group; a (C₁-C₆) alkoxycarbonyl group; or a phenylcarbonyl group);
(c22) an R¹⁰ON═C(R⁹) group (wherein R⁹ and R¹⁰ are as defined above);
(c24) a (C₁-C₆) alkoxycarbonyl group;
(c25) an R⁹(R¹⁰)N carbonyl group (wherein R⁹ and R¹⁰ are as defined above);
(c27) an aryl group having, on the ring, 1 to 5 substituting groups independently selected from the group consisting of a halogen atom, a (C₁-C₆) alkyl group, a halo (C₁-C₆) alkyl group, a (C₁-C₆) alkoxy group, a halo (C₁-C₆) alkoxy group, a (C₁-C₆) alkylthio group, a halo (C₁-C₆) alkylthio group, a (C₁-C₆) alkylsulfinyl group, a halo (C₁-C₆) alkylsulfinyl group, a (C₁-C₆) alkylsulfonyl group and a halo (C₁-C₆) alkylsulfonyl group; or
(c36) a (C₁-C₆) alkoxycarbonyl NH group,
and more preferably
(c1) a hydrogen atom;
(c2) a halogen atom;
(c8) a (C₁-C₆) alkoxy group;
(c9) a halo (C₁-C₆) alkyl group; or
(c11) a halo (C₁-C₆) alkoxy group.

R⁴ is preferably

(d1) a hydrogen atom;
(d2) a halogen atom;
(d3) a (C₁-C₆) alkyl group;
(d4) a (C₃-C₆) cycloalkyl group;
(d7) a (C₁-C₆) alkoxy group;
(d12) a halo (C₁-C₆) alkoxy group;
(d13) a cyano group;
(d14) an R⁹(R¹⁰)N group (wherein R⁹ and R¹⁰ are as defined above);
(d15) a (C₁-C₆) alkylthio group;
(d16) a (C₁-C₆) alkylsulfinyl group;
(d17) a (C₁-C₆) alkylsulfonyl group;
(d18) a (C₁-C₆) alkylthio group;
(d19) a formyl group;
(d20) a (C₁-C₆) alkoxycarbonyl group;
(d21) an R⁹(R¹⁰)N carbonyl group (wherein R⁹ and R¹⁰ are as defined above); or
(d22) an R¹⁰ON═C(R⁹) group (wherein R⁹ and R¹⁰ are as defined above),
and more preferably
(d1) a hydrogen atom or
(d2) a halogen atom.

G⁶ is preferably N—R⁵ or O.

R⁵ is preferably (e2) a (C₁-C₆) alkyl group.

The compounds of the present invention can be produced according to, for example, the production methods described below, which are non-limiting examples.

### Production Method 1

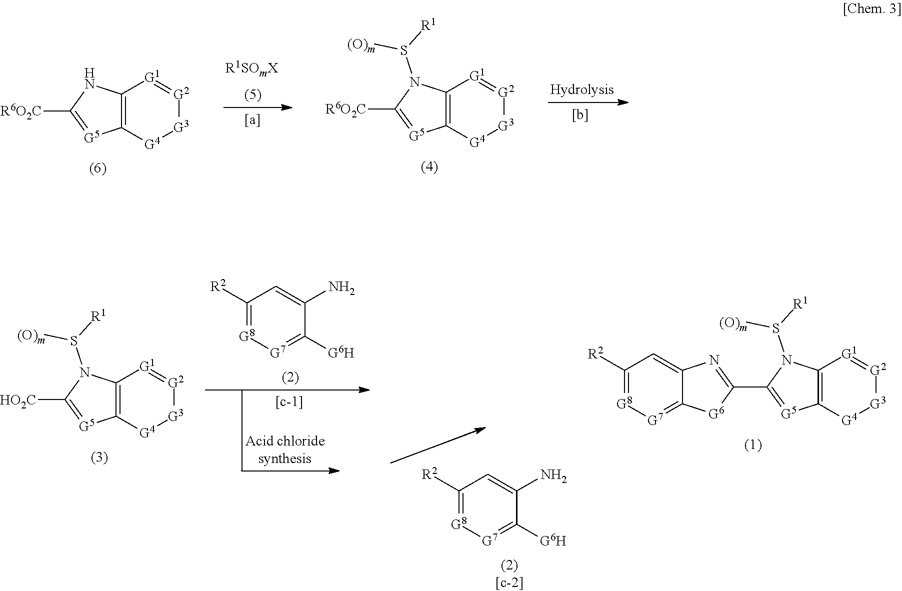

(In the formula, R², R², R⁶, G², G², G³, G⁴, G⁵, G⁶, G⁷, G⁸ and m are as defined above, and X represents a halogen atom.)

The compound represented by the general formula (1) of the present invention can be produced through the steps [a], [b] and [c-1] or [c-2] described below.

Step [a]

A step of reacting the compound represented by the general formula (6) with the compound represented by the general formula (5), to produce the compound represented by the general formula (4).

Step [b]

A step of hydrolyzing the compound represented by the general formula (4), to produce the compound represented by the general formula (3).

Step [c-1]

A step of reacting the compound represented by the general formula (3) with the compound represented by the general formula (2) in the presence of a condensing agent for amide condensation, to produce the compound represented by the general formula (1).

Step [c-2]

A step of synthesizing an acid halide from the compound represented by the general formula (3) and reacting the acid halide with the compound represented by the general formula (2), to produce the compound represented by the general formula (1).

Production Method at Step [a]

The compound represented by the general formula (4) can be produced by reacting the compound represented by the general formula (6) with the compound represented by the general formula (5) in the presence of a base and an inert solvent.

Examples of the base that can be used in this reaction include alkyllithiums such as sec-butyllithium and tert-butyllithium; organometallic compounds such as lithium hexamethyldisilazane and sodium hexamethyldisilazane; alkoxides such as sodium tert-butoxide and potassium tert-butoxide; and metal hydrides such as sodium hydride and potassium hydride. The amount of the base used is usually in the range of a 1- to 10-fold molar amount relative to the compound represented by the general formula (6).

The inert solvent used in this reaction may be any solvent that does not markedly inhibit the progress of the reaction, and examples include straight-chain or cyclic saturated hydrocarbons such as pentane, hexane and cyclohexane; aromatic hydrocarbons such as benzene, toluene and xylene; straight-chain or cyclic ethers such as diethyl ether, methyl tert-butyl ether, dioxane and tetrahydrofuran; and aprotic polar solvents such as N,N-dimethylformamide, N,N-dimethylacetamide, dimethyl sulfoxide and 1,3-dimethyl-2-imidazolidinone. One of these inert solvents may be used alone, and also two or more of them may be used as a mixture. The amount of the inert solvent used is usually selected as appropriate from the range of 0.1 to 100 L relative to 1 mol of the compound represented by the general formula (2).

Since this reaction is an equimolar reaction of the compounds, they are basically used in equimolar amounts, but either of them may be used in an excess amount. The reaction temperature in this reaction is usually in the range of about −78° C. to the boiling point of the solvent used. The reaction time varies with the reaction scale, the reaction temperature and the like and is not the same in every case, but is usually selected as appropriate from the range of a few minutes to 48 hours. After the reaction is completed, the compound of interest is isolated from the post-reaction mixture by the usual method. As needed, recrystallization, column chromatography, etc. can be employed for the purification of the compound of interest.

Production Method at Step [b]

The compound represented by the general formula (3) can be produced by hydrolyzing the compound represented by the general formula (4) in the presence of a base, an inert solvent and water.

Examples of the base that can be used in this reaction include hydroxides such as lithium hydroxide, sodium hydroxide, potassium hydroxide and calcium hydroxide; and alkoxides such as sodium methoxide, sodium ethoxide, sodium tert-butoxide and potassium tert-butoxide. The amount of the base used is usually in the range of a 1- to 10-fold molar amount relative to the compound represented by the general formula (4).

The inert solvent used in this reaction may be any solvent that does not markedly inhibit the reaction, and examples include alcohols such as methanol, ethanol, propanol, butanol and 2-propanol; straight-chain or cyclic ethers such as diethyl ether, tetrahydrofuran (THF) and dioxane; aprotic polar solvents such as N,N-dimethylformamide, N,N-dimethylacetamide, dimethyl sulfoxide and 1,3-dimethyl-2-imidazolidinone; and water. One of these inert solvents may be used alone, or two or more of them may be used as a mixture. The amount of the inert solvent used is usually selected as appropriate from the range of 0.1 to 100 L relative to 1 mol of the compound represented by the general formula (4).

The reaction temperature in this reaction is usually in the range of about 0° C. to the boiling point of the solvent used. The reaction time varies with the reaction scale, the reaction temperature and the like and is not the same in every case, but is usually selected as appropriate from the range of a few minutes to 48 hours. After the reaction is completed, the compound of interest is isolated from the post-reaction mixture by the usual method. As needed, recrystallization, column chromatography, etc. can be employed for the purification of the compound of interest.

Production Method at Step [c-1]

The compound represented by the general formula (3) is reacted with the compound represented by the general formula (2) in the presence of a condensing agent, a base and an inert solvent. The resulting intermediate is optionally isolated, and is allowed to react under acid conditions, to yield the compound represented by the general formula (1).

Examples of the condensing agent used in this reaction include 1-ethyl-3-(3-dimethylaminopropyl)carbodiimide hydrochloride (EDC), diethyl phosphorocyanidate (DEPC), carbonyldiimidazole (CDI), 1,3-dicyclohexylcarbodiimide (DCC), chlorocarbonic esters and 2-chloro-1-methylpyridinium iodide. The amount of the condensing agent used is usually selected as appropriate from the range of a 1- to 2-fold molar amount relative to the compound represented by the general formula (3).

Examples of the base that can be used in this reaction include carbonates such as lithium carbonate, lithium hydrogen carbonate, sodium carbonate, sodium hydrogen carbonate, potassium carbonate, potassium hydrogen carbonate, calcium carbonate and magnesium carbonate; acetates such as lithium acetate, sodium acetate and potassium acetate; and organic bases such as pyridine, picoline, lutidine, triethylamine, tributylamine and diisopropylethylamine. The amount of the base used is usually selected as appropriate from the range of a 1- to 10-fold molar amount relative to the compound represented by the general formula (3).

The inert solvent used in this reaction may be any solvent that does not markedly inhibit the reaction, and examples include aromatic hydrocarbons such as benzene, toluene and xylene; halogenated hydrocarbons such as methylene chloride, chloroform and carbon tetrachloride; halogenated aromatic hydrocarbons such as chlorobenzene and dichlorobenzene; straight-chain or cyclic ethers such as diethyl ether, tetrahydrofuran (THF) and dioxane; nitriles such as acetonitrile and propionitrile; esters such as methyl acetate; and aprotic polar solvents such as N,N-dimethylformamide, N,N-dimethylacetamide, dimethyl sulfoxide and 1,3-dimethyl-2-imidazolidinone. One of these inert solvents may be used alone, or two or more of them may be used as a mixture. The amount of the inert solvent used is usually selected as appropriate from the range of 0.1 to 100 L relative to 1 mol of the compound represented by the general formula (3).

Since this reaction is an equimolar reaction of the compounds, they are basically used in equimolar amounts, but either of them may be used in an excess amount. The reaction temperature in this reaction is usually in the range of about 0° C. to the boiling point of the solvent used. The reaction time varies with the reaction scale, the reaction temperature and the like and is not the same in every case, but is usually selected as appropriate from the range of a few minutes to 48 hours.

After the reaction is completed, the resulting intermediate is optionally isolated from the post-reaction mixture, and is allowed to react in the presence of an acid and an inert solvent, to yield the compound represented by the general formula (1).

Examples of the acid used in this reaction include inorganic acids such as hydrochloric acid, sulfuric acid and nitric acid; organic acids such as formic acid, acetic acid, propionic acid, trifluoroacetic acid and benzoic acid; sulfonic acids such as methanesulfonic acid and trifluoromethanesulfonic acid; and phosphoric acid. The amount of the acid used is usually selected as appropriate from the range of a 0.01- to 10-fold molar amount relative to the compound represented by the general formula (3).

The inert solvent used in this reaction may be any solvent that does not markedly inhibit the progress of the reaction, and examples include aromatic hydrocarbons such as benzene, toluene and xylene; halogenated hydrocarbons such as methylene chloride, chloroform and carbon tetrachloride; halogenated aromatic hydrocarbons such as chlorobenzene and dichlorobenzene; straight-chain or cyclic ethers such as diethyl ether, methyl tert-butyl ether, dioxane and tetrahydrofuran; esters such as ethyl acetate; amides such as dimethylformamide and dimethylacetamide; ketones such as acetone and methyl ethyl ketone; and aprotic polar solvents such as dimethyl sulfoxide and 1,3-dimethyl-2-imidazolidinone. One of these inert solvents may be used alone, or two or more of them may be used as a mixture. The amount of the inert solvent used is usually selected as appropriate from the range of 0.1 to 100 L relative to 1 mol of the compound represented by the general formula (3).

The reaction temperature in this reaction is usually in the range of about 0° C. to the boiling point of the solvent used. The reaction time varies with the reaction scale, the reaction temperature and the like and is not the same in every case, but is usually selected as appropriate from the range of a few minutes to 48 hours. After the reaction is completed, the compound of interest is isolated from the post-reaction mixture by the usual method. As needed, recrystallization, column chromatography, etc. can be employed for the purification of the compound of interest.

Production Method at Step [c-2]

The compound represented by the general formula (3) is reacted with a halogenating agent in the presence of an inert solvent to give an acid halide. The acid halide is reacted with the compound represented by the general formula (2) in the presence of an inert solvent and a base to give an intermediate. The obtained intermediate is optionally isolated, and is allowed to react under acid conditions, to yield the compound represented by the general formula (1).

Examples of the halogenating agent used in this reaction include phosphorus pentachloride, thionyl chloride, phosphorus oxychloride and oxalyl chloride. The amount of the halogenating agent used is usually selected as appropriate from the range of a 1- to 1.5-fold molar amount relative to the compound represented by the general formula (3).

Examples of the base that can be used in this reaction include carbonates such as lithium carbonate, lithium hydrogen carbonate, sodium carbonate, sodium hydrogen carbonate, potassium carbonate, potassium hydrogen carbonate, calcium carbonate and magnesium carbonate; acetates such as lithium acetate, sodium acetate and potassium acetate; and organic bases such as pyridine, picoline, lutidine, triethylamine, tributylamine and diisopropylethylamine. The amount of the base used is usually selected as appropriate from the range of a 1- to 10-fold molar amount relative to the compound represented by the general formula (3).

The inert solvent used in this reaction may be any solvent that does not markedly inhibit the reaction, and examples include aromatic hydrocarbons such as benzene, toluene and xylene; halogenated hydrocarbons such as methylene chloride, chloroform and carbon tetrachloride; halogenated aromatic hydrocarbons such as chlorobenzene and dichlorobenzene; straight-chain or cyclic ethers such as diethyl ether, tetrahydrofuran (THF) and dioxane; nitriles such as acetonitrile and propionitrile; esters such as methyl acetate; and polar solvents such as N,N-dimethylformamide, N,N-dimethylacetamide, dimethyl sulfoxide and 1,3-dimethyl-2-imidazolidinone. One of these inert solvents may be used alone, or two or more of them may be used as a mixture. The amount of the inert solvent used is usually selected as appropriate from the range of 0.1 to 100 L relative to 1 mol of the compound represented by the general formula (3).

Since this reaction is an equimolar reaction of the compounds, they are basically used in equimolar amounts, but either of them may be used in an excess amount. The reaction temperature in this reaction is usually in the range of about 0° C. to the boiling point of the solvent used. The reaction time varies with the reaction scale, the reaction temperature and the like and is not the same in every case, but is usually selected as appropriate from the range of a few minutes to 48 hours.

After the reaction is completed, the resulting intermediate is optionally isolated from the post-reaction mixture, and is allowed to react in the presence of an acid and an inert solvent, to yield the compound of interest.

Examples of the acid used in this reaction include inorganic acids such as hydrochloric acid, sulfuric acid and nitric acid; organic acids such as formic acid, acetic acid, propionic acid, trifluoroacetic acid and benzoic acid; sulfonic acids such as methanesulfonic acid and trifluoromethanesulfonic acid; and phosphoric acid. The amount of the acid used is usually selected as appropriate from the range of a 0.01- to 10-fold molar amount relative to the compound represented by the general formula (3).

The inert solvent used in this reaction may be any solvent that does not markedly inhibit the progress of the reaction, and examples include aromatic hydrocarbons such as benzene, toluene and xylene; halogenated hydrocarbons such as methylene chloride, chloroform and carbon tetrachloride; halogenated aromatic hydrocarbons such as chlorobenzene and dichlorobenzene; straight-chain or cyclic ethers such as diethyl ether, methyl tert-butyl ether, dioxane and tetrahydrofuran; esters such as ethyl acetate; amides such as dimethylformamide and dimethylacetamide; ketones such as acetone and methyl ethyl ketone; and aprotic polar solvents such as dimethyl sulfoxide and 1,3-dimethyl-2-imidazolidinone. One of these inert solvents may be used alone, or two or more of them may be used as a mixture. The amount of the inert solvent used is usually selected as appropriate from the range of 0.1 to 100 L relative to 1 mol of the compound represented by the general formula (3).

The reaction temperature in this reaction is usually in the range of about 0° C. to the boiling point of the solvent used. The reaction time varies with the reaction scale, the reaction temperature and the like and is not the same in every case, but is usually selected as appropriate from the range of a few minutes to 48 hours. After the reaction is completed, the compound of interest is isolated from the post-reaction mixture by the usual method. As needed, recrystallization, column chromatography, etc. can be employed for the purification of the compound of interest.

### Production Method 2

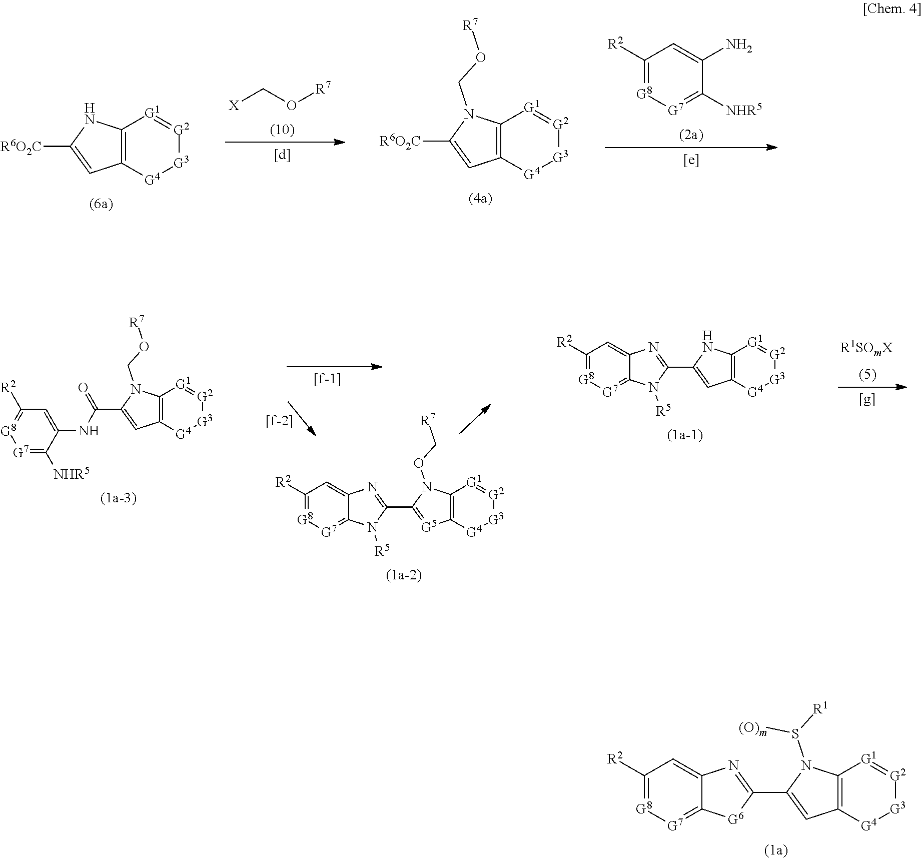

(In the formula, R¹, R², R⁵, R⁶, G¹, G², G³, G⁴, G⁷, G⁸, m and X are as defined above, and R⁷ represents a (C₁-C₆) alkyl group or a phenyl (C₁-C₆) alkyl group.)

The compound represented by the general formula (1a) of the present invention can be produced through the steps [d], [e], [g] and [f-1] or [f-2] described below.

Step [d]

A step of reacting the compound represented by the general formula (6a) with the compound represented by the general formula (10), to produce the compound represented by the general formula (4a).

Step [e]

A step of reacting the compound represented by the general formula (4a) with the compound represented by the general formula (2a), to produce the compound represented by the general formula (1a-3).

Step [f-1]

A step of subjecting the compound represented by the general formula (1a-3) to cyclization and concomitant deprotection of the protective group on the nitrogen atom, to produce the compound represented by the general formula (1a-1).

Step [f-2]

A step of subjecting the compound represented by the general formula (1a-3) to cyclization to produce the compound represented by the general formula (1a-2), followed by deprotection of the protective group on the nitrogen atom, to produce the compound represented by the general formula (1a-1).

Step [g]

A step of reacting the compound represented by the general formula (1a-1) with the compound represented by the general formula (5), to produce the compound represented by the general formula (1a).

Production Method at Step [d]

The compound represented by the general formula (4a) can be produced by reacting the compound represented by the general formula (6a) with the compound represented by the general formula (10) in the presence of a base and an inert solvent.

Examples of the base that can be used in this reaction include alkyllithiums such as methyllithium, n-butyllithium, sec-butyllithium and tert-butyllithium; organometallic compounds such as lithium hexamethyldisilazane and sodium hexamethyldisilazane; hydroxides such as lithium hydroxide, sodium hydroxide, potassium hydroxide and calcium hydroxide; carbonates such as lithium carbonate, lithium hydrogen carbonate, sodium carbonate, sodium hydrogen carbonate, potassium carbonate, potassium hydrogen carbonate, calcium carbonate and magnesium carbonate; acetates such as lithium acetate, sodium acetate and potassium acetate; alkoxides such as sodium methoxide, sodium ethoxide, sodium tert-butoxide and potassium tert-butoxide; and metal hydrides such as sodium hydride and potassium hydride. The amount of the base used is usually selected as appropriate from the range of a 1- to 5-fold molar amount relative to the compound represented by the general formula (6a).

The inert solvent used in this reaction may be any solvent that does not markedly inhibit the progress of the reaction, and examples include straight-chain or cyclic saturated hydrocarbons such as pentane, hexane and cyclohexane; aromatic hydrocarbons such as benzene, toluene and xylene; straight-chain or cyclic ethers such as diethyl ether, methyl tert-butyl ether, dioxane and tetrahydrofuran; and aprotic polar solvents such as N,N-dimethylformamide, N,N-dimethylacetamide, dimethyl sulfoxide and 1,3-dimethyl-2-imidazolidinone. One of these inert solvents may be used alone, or two or more of them may be used as a mixture. The amount of the inert solvent used is usually selected as appropriate from the range of 0.1 to 100 L relative to 1 mol of the compound represented by the general formula (6a).

Since this reaction is an equimolar reaction of the compounds, they are basically used in equimolar amounts, but either of them may be used in an excess amount. The reaction temperature in this reaction is usually in the range of about 0° C. to the boiling point of the solvent used. The reaction time varies with the reaction scale, the reaction temperature and the like and is not the same in every case, but is usually selected as appropriate from the range of a few minutes to 48 hours. After the reaction is completed, the compound of interest is isolated from the post-reaction mixture by the usual method. As needed, recrystallization, column chromatography, etc. can be employed for the purification of the compound of interest.

Production Method at Step [e]

The compound represented by the general formula (1a-3) can be produced by reacting the compound represented by the general formula (4a) with the compound represented by the general formula (2a) in the presence of a base and an inert solvent.

Examples of the base that can be used in this reaction include alkyllithiums such as sec-butyllithium and tert-butyllithium; organometallic compounds such as lithium hexamethyldisilazane and sodium hexamethyldisilazane; alkoxides such as sodium tert-butoxide and potassium tert-butoxide; and metal hydrides such as sodium hydride and potassium hydride. The amount of the base used is usually selected as appropriate from the range of a 1- to 5-fold molar amount relative to the compound represented by the general formula (4a).

The inert solvent used in this reaction may be any solvent that does not markedly inhibit the progress of the reaction, and examples include straight-chain or cyclic saturated hydrocarbons such as pentane, hexane and cyclohexane; aromatic hydrocarbons such as benzene, toluene and xylene; straight-chain or cyclic ethers such as diethyl ether, methyl tert-butyl ether, dioxane and tetrahydrofuran; and aprotic polar solvents such as N,N-dimethylformamide, N,N-dimethylacetamide, dimethyl sulfoxide and 1,3-dimethyl-2-imidazolidinone. One of these inert solvents may be used alone, or two or more of them may be used as a mixture. The amount of the inert solvent used is usually selected as appropriate from the range of 0.1 to 100 L relative to 1 mol of the compound represented by the general formula (4a).

Since this reaction is an equimolar reaction of the compounds, they are basically used in equimolar amounts, but either of them may be used in an excess amount. The reaction temperature in this reaction is usually in the range of about 0° C. to the boiling point of the solvent used. The reaction time varies with the reaction scale, the reaction temperature and the like and is not the same in every case, but is usually selected as appropriate from the range of a few minutes to 48 hours. After the reaction is completed, the compound of interest is isolated from the post-reaction mixture by the usual method. As needed, recrystallization, column chromatography, etc. can be employed for the purification of the compound of interest.

Production Method at Step [f-1]

The compound represented by the general formula (1a-1) can be produced by allowing the compound represented by the general formula (1a-3) to react in the presence of an acid and an inert solvent.

Examples of the acid that can be used in this reaction include inorganic acids such as hydrochloric acid, sulfuric acid and nitric acid; and organic acids such as methanesulfonic acid, p-toluenesulfonic acid, trifluoroacetic acid and trifluoromethanesulfonic acid. The amount of the acid used is usually selected as appropriate from the range of a 0.01- to 10-fold molar amount relative to the compound represented by the general formula (1a-3).

The inert solvent used in this reaction may be any solvent that does not markedly inhibit the progress of the reaction, and examples include alcohols such as methanol, ethanol, propanol, butanol and 2-propanol; straight-chain or cyclic saturated hydrocarbons such as pentane, hexane and cyclohexane; aromatic hydrocarbons such as benzene, toluene and xylene; halogenated hydrocarbons such as methylene chloride, chloroform and carbon tetrachloride; halogenated aromatic hydrocarbons such as chlorobenzene and dichlorobenzene; straight-chain or cyclic ethers such as diethyl ether, methyl tert-butyl ether, dioxane, tetrahydrofuran and cyclopentyl methyl ether; esters such as ethyl acetate; nitriles such as acetonitrile and propionitrile; amides such as dimethylformamide and dimethylacetamide; ketones such as acetone and methyl ethyl ketone; aprotic polar solvents such as dimethyl sulfoxide and 1,3-dimethyl-2-imidazolidinone; and water. One of these inert solvents may be used alone, or two or more of them may be used as a mixture. The amount of the inert solvent used is usually selected as appropriate from the range of 0.1 to 100 L relative to 1 mol of the compound represented by the general formula (1a-3).

The reaction temperature in this reaction is usually in the range of about 0° C. to the boiling point of the solvent used. The reaction time varies with the reaction scale, the reaction temperature and the like and is not the same in every case, but is usually selected as appropriate from the range of a few minutes to 48 hours. After the reaction is completed, the compound of interest is isolated from the post-reaction mixture by the usual method. As needed, recrystallization, column chromatography, etc. can be employed for the purification of the compound of interest.

Production Method at Step [f-2]

The compound represented by the general formula (1a-3) is allowed to react in the presence of an acid and an inert solvent to give the compound represented by the general formula (1a-2). The compound represented by the general formula (1a-2) is allowed to react in the presence of an acid and an inert solvent, to yield the compound represented by the general formula (1a-1).

Examples of the acid that can be used in this reaction include inorganic acids such as hydrochloric acid, sulfuric acid, nitric acid and phosphoric acid; and organic acids such as acetic acid, methanesulfonic acid, p-toluenesulfonic acid, trifluoroacetic acid and trifluoromethanesulfonic acid. The amount of the acid used is usually selected as appropriate from the range of a 0.01- to 10-fold molar amount relative to the compound represented by the general formula (1a-3).

The inert solvent used in this reaction may be any solvent that does not markedly inhibit the progress of the reaction, and examples include alcohols such as methanol, ethanol, propanol, butanol and 2-propanol; straight-chain or cyclic saturated hydrocarbons such as pentane, hexane and cyclohexane; aromatic hydrocarbons such as benzene, toluene and xylene; halogenated hydrocarbons such as methylene chloride, chloroform and carbon tetrachloride; halogenated aromatic hydrocarbons such as chlorobenzene and dichlorobenzene; straight-chain or cyclic ethers such as diethyl ether, methyl tert-butyl ether, dioxane, tetrahydrofuran and cyclopentyl methyl ether; esters such as ethyl acetate; nitriles such as acetonitrile and propionitrile; amides such as dimethylformamide and dimethylacetamide; ketones such as acetone and methyl ethyl ketone; aprotic polar solvents such as dimethyl sulfoxide and 1,3-dimethyl-2-imidazolidinone; and water. One of these inert solvents may be used alone, or two or more of them may be used as a mixture. The amount of the inert solvent used is usually selected as appropriate from the range of 0.1 to 100 L relative to 1 mol of the compound represented by the general formula (1a-3).

The reaction temperature in this reaction is usually in the range of about 0° C. to the boiling point of the solvent used. The reaction time varies with the reaction scale, the reaction temperature and the like and is not the same in every case, but is usually selected as appropriate from the range of a few minutes to 48 hours. After the reaction is completed, the compound of interest is isolated from the post-reaction mixture by the usual method. As needed, recrystallization, column chromatography, etc. can be employed for the purification of the compound of interest.

Production Method of General Formula (1a) from General Formula (1a-1)

The compound represented by the general formula (1a) can be produced by subjecting the compound represented by the general formula (1a-1) to the reaction according to the production method at step [a] described in production method 1.

### Production Method 3

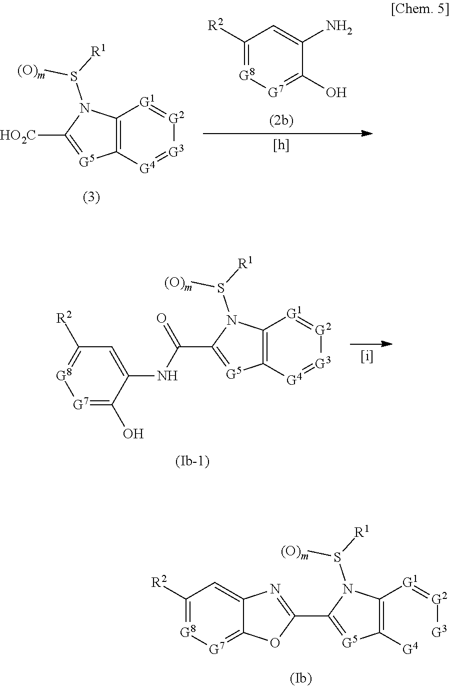

(In the formula, R¹, R², G¹, G², G³, G⁴, G⁵, G⁷, G⁸ and m are as defined above.)

The compound represented by the general formula (1b) of the present invention can be produced through the steps [h] and [i] described below.

Step [h]

A step of reacting the compound represented by the general formula (3) with the compound represented by the general formula (2b), to produce the compound represented by the general formula (1b-1).

Step [i]

A step of cyclizing the compound represented by the general formula (1b-1), to produce the compound represented by the general formula (1b).

Production Method at Step [h]

The compound represented by the general formula (1b-1) can be produced by reacting the compound represented by the general formula (3) with the compound represented by the general formula (2b) in the presence of a condensing agent, a base and an inert solvent.

Examples of the condensing agent used in this reaction include, but are not limited to, diethyl phosphorocyanidate (DEPC), carbonyldiimidazole (CDI), 1,3-dicyclohexylcarbodiimide (DCC), 1-ethyl-3-(3-dimethylaminopropyl)-carbodiimide (EDC), chlorocarbonic esters and 2-chloro-1-methylpyridinium iodide. The amount of the condensing agent used is usually selected as appropriate from the range of a 1- to 2-fold molar amount relative to the compound represented by the general formula (3).

Examples of the base that can be used in this reaction include carbonates such as lithium carbonate, lithium hydrogen carbonate, sodium carbonate, sodium hydrogen carbonate, potassium carbonate, potassium hydrogen carbonate, calcium carbonate and magnesium carbonate; acetates such as lithium acetate, sodium acetate and potassium acetate; and organic bases such as pyridine, picoline, lutidine, triethylamine, tributylamine and diisopropylethylamine. The amount of the base used is usually selected as appropriate from the range of a 1- to 10-fold molar amount relative to the compound represented by the general formula (3).

The inert solvent used in this reaction may be any solvent that does not markedly inhibit the reaction, and examples include aromatic hydrocarbons such as benzene, toluene and xylene; halogenated hydrocarbons such as methylene chloride, chloroform and carbon tetrachloride; halogenated aromatic hydrocarbons such as chlorobenzene and dichlorobenzene; straight-chain or cyclic ethers such as diethyl ether, tetrahydrofuran (THF) and dioxane; nitriles such as acetonitrile and propionitrile; esters such as methyl acetate; and aprotic polar solvents such as N,N-dimethylformamide, N,N-dimethylacetamide, dimethyl sulfoxide and 1,3-dimethyl-2-imidazolidinone. One of these inert solvents may be used alone, or two or more of them may be used as a mixture. The amount of the inert solvent used is usually selected as appropriate from the range of 0.1 to 100 L relative to 1 mol of the compound represented by the general formula (3).

Since this reaction is an equimolar reaction of the compounds, they are basically used in equimolar amounts, but either of them may be used in an excess amount. The reaction temperature in this reaction is usually in the range of about 0° C. to the boiling point of the solvent used. The reaction time varies with the reaction scale, the reaction temperature and the like and is not the same in every case, but is usually selected as appropriate from the range of a few minutes to 48 hours. After the reaction is completed, the compound of interest is isolated from the post-reaction mixture by the usual method. As needed, recrystallization, column chromatography, etc. can be employed for the purification of the compound of interest.

Production Method of General Formula (1b) from General Formula (1b-1)

The compound represented by the general formula (1b) can be produced by allowing the compound represented by the general formula (1b-1) to react in the presence of an azodicarboxylic acid ester, triphenylphosphine and an inert solvent.

Examples of the azodicarboxylic acid ester that can be used in this reaction include, but are not limited to, diethyl azodicarboxylate (DEAD), diisopropyl azodicarboxylate and bis(2-methoxyethyl) azodicarboxylate. The amount of the azodicarboxylic acid ester used is usually selected as appropriate from the range of a 1- to 5-fold molar amount relative to the compound represented by the general formula (3).

The inert solvent used in this reaction may be any solvent that does not markedly inhibit the reaction, and examples include aromatic hydrocarbons such as benzene, toluene and xylene; halogenated hydrocarbons such as methylene chloride, chloroform and carbon tetrachloride; halogenated aromatic hydrocarbons such as chlorobenzene and dichlorobenzene; straight-chain or cyclic ethers such as diethyl ether, tetrahydrofuran (THF) and dioxane; nitriles such as acetonitrile and propionitrile; esters such as methyl acetate; and polar solvents such as N,N-dimethylformamide, N,N-dimethylacetamide, dimethyl sulfoxide and 1,3-dimethyl-2-imidazolidinone. One of these inert solvents may be used alone, or two or more of them may be used as a mixture. The amount of the inert solvent used is usually selected as appropriate from the range of 0.1 to 100 L relative to 1 mol of the compound represented by the general formula (1b-1).

The reaction temperature in this reaction is usually in the range of about 0° C. to the boiling point of the solvent used. The reaction time varies with the reaction scale, the reaction temperature and the like and is not the same in every case, but is usually selected as appropriate from the range of a few minutes to 48 hours. After the reaction is completed, the compound of interest is isolated from the post-reaction mixture by the usual method. As needed, recrystallization, column chromatography, etc. can be employed for the purification of the compound of interest.

### Production Method (1) of Intermediate

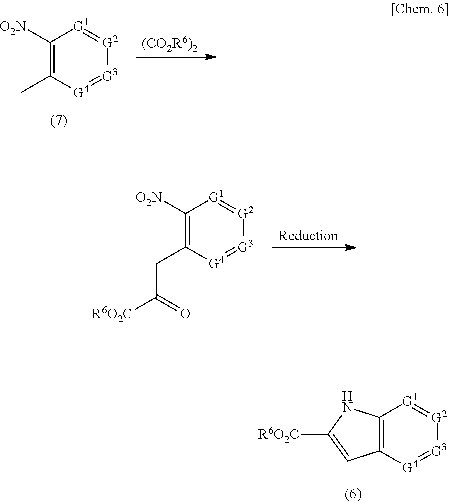

(In the formula, G¹, G², G³ and G⁴ are as defined above, and R⁶ represents a (C₁-C₆) alkyl group.)
Production Method of General Formula (6) from General Formula (7)

The compound represented by the general formula (6) can be produced by subjecting the compound represented by the general formula (7) to the reaction according to the method described in the literature (J. O. C., 2001, 66, 3474.).

### Production Method (2) of Intermediate

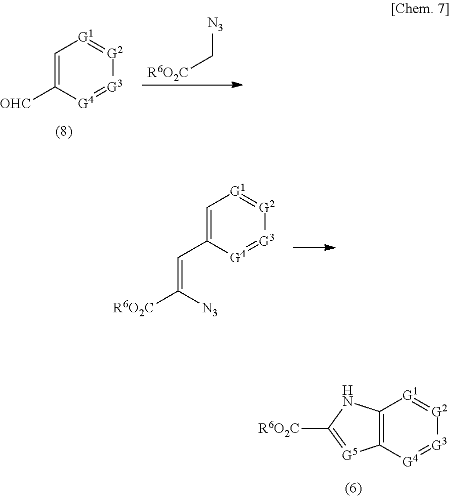

(In the formula, G¹, G², G³, G⁴ and R⁶ are as defined above.)
Production Method of General Formula (6) from General Formula (8)

The compound represented by the general formula (6) can be produced by subjecting the compound represented by the general formula (8) to the reaction according to the method described in the literature (J. Med. Chem., 2004, 47, 6270.).

### Production Method (3) of Intermediate

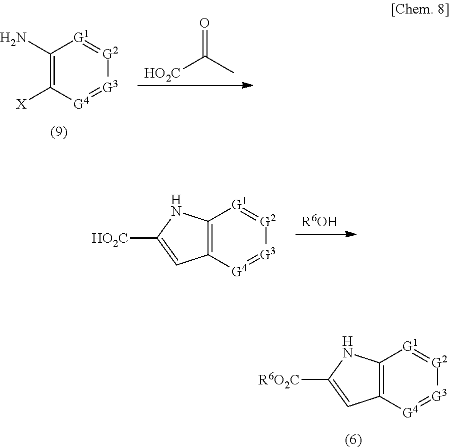

Production Method of General Formula (6) from General Formula (9)

The compound represented by the general formula (6) can be produced by subjecting the compound represented by the general formula (9) to the reaction according to the method described in the literature (WO 2014/073627).

Representative examples of the compounds represented by the general formula (1) of the present invention are shown in Tables 1 to 10 below, but the present invention is not limited thereto.

In the tables below, “Me” represents a methyl group, “Et” represents an ethyl group, “Pr” represents a propyl group, “Ac” represents an acetyl group, “Ph” represents a phenyl group, and “c-” represents cyclo. Shown in the column of “Physical property” is a melting point (° C.), a refractive index n_{D} (the figure in parentheses is the measurement temperature (° C.)) or “NMR”. NMR data are shown in Table 11.

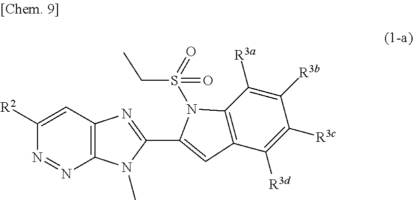

TABLE 1

| Compound | | | | | | Physical |
|---|---|---|---|---|---|---|
| No. | R^{3a} | R^{3b} | R^{3c} | R^{3d} | R² | property |
| 1-1 | H | H | H | H | CF₃ | 158-159 |
| 1-2 | Cl | H | H | H | CF₃ | |
| 1-3 | H | Cl | H | H | CF₃ | |
| 1-4 | H | H | Cl | H | CF₃ | |
| 1-5 | H | H | H | Cl | CF₃ | |
| 1-6 | Me | H | H | H | CF₃ | |
| 1-7 | H | Me | H | H | CF₃ | |
| 1-8 | H | H | Me | H | CF₃ | |
| 1-9 | H | H | H | Me | CF₃ | |
| 1-10 | H | CF₃ | H | H | CF₃ | |
| 1-11 | H | H | CF₃ | H | CF₃ | |
| 1-12 | H | CN | H | H | CF₃ | |
| 1-13 | H | H | CN | H | CF₃ | |
| 1-14 | H | OMe | H | H | CF₃ | |
| 1-15 | H | H | OMe | H | CF₃ | |
| 1-16 | H | NH₂ | H | H | CF₃ | |
| 1-17 | H | H | NH₂ | H | CF₃ | |
| 1-18 | H | NMe₂ | H | H | CF₃ | |
| 1-19 | H | H | NMe₂ | H | CF₃ | |
| 1-20 | H | NHAc | H | H | CF₃ | |
| 1-21 | H | H | NHAc | H | CF₃ | |
| 1-22 | H | SMe | H | H | CF₃ | |
| 1-23 | H | H | SMe | H | CF₃ | |
| 1-24 | H | SCF₃ | H | H | CF₃ | |
| 1-25 | H | H | SCF₃ | H | CF₃ | |
| 1-26 | H | CHO | H | H | CF₃ | |
| 1-27 | H | CO₂Et | H | H | CF₃ | |
| 1-28 | H | CONHMe | H | H | CF₃ | |
| 1-29 | H | CH═NOEt | H | H | CF₃ | |
| 1-30 | H | c-Pr | H | H | CF₃ | |
| 1-31 | H | Br | H | H | CF₃ | |
| 1-32 | H | I | H | H | CF₃ | |
| 1-33 | H | H | H | H | CF₂CF₃ | 145-146 |
| 1-34 | Cl | H | H | H | CF₂CF₃ | |
| 1-35 | H | Cl | H | H | CF₂CF₃ | 92-94 |
| 1-36 | H | H | Cl | H | CF₂CF₃ | NMR |
| 1-37 | H | H | H | Cl | CF₂CF₃ | |
| 1-38 | Me | H | H | H | CF₂CF₃ | |
| 1-39 | H | Me | H | H | CF₂CF₃ | |
| 1-40 | H | H | Me | H | CF₂CF₃ | |
| 1-41 | H | H | H | Me | CF₂CF₃ | |
| 1-42 | H | CF₃ | H | H | CF₂CF₃ | |
| 1-43 | H | H | CF₃ | H | CF₂CF₃ | 201-202 |
| 1-44 | H | CN | H | H | CF₂CF₃ | |
| 1-45 | H | H | CN | H | CF₂CF₃ | |
| 1-46 | H | OMe | H | H | CF₂CF₃ | |
| 1-47 | H | H | OMe | H | CF₂CF₃ | |
| 1-48 | H | NH₂ | H | H | CF₂CF₃ | |
| 1-49 | H | H | NH₂ | H | CF₂CF₃ | |
| 1-50 | H | NMe₂ | H | H | CF₂CF₃ | |
| 1-51 | H | H | NMe₂ | H | CF₂CF₃ | |
| 1-52 | H | NHAc | H | H | CF₂CF₃ | |
| 1-53 | H | H | NHAc | H | CF₂CF₃ | |
| 1-54 | H | SMe | H | H | CF₂CF₃ | |
| 1-55 | H | H | SMe | H | CF₂CF₃ | |
| 1-56 | H | SCF₃ | H | H | CF₂CF₃ | |
| 1-57 | H | H | SCF₃ | H | CF₂CF₃ | |
| 1-58 | H | CHO | H | H | CF₂CF₃ | |
| 1-59 | H | CO₂Et | H | H | CF₂CF₃ | |
| 1-60 | H | CONHMe | H | H | CF₂CF₃ | |
| 1-61 | H | CH═NOEt | H | H | CF₂CF₃ | |
| 1-62 | H | c-Pr | H | H | CF₂CF₃ | |
| 1-63 | H | Br | H | H | CF₂CF₃ | 106-110 |
| 1-64 | H | I | H | H | CF₂CF₃ | |
| 1-65 | H | F | H | H | CF₂CF₃ | 82-86 |
| 1-66 | H | Cl | Br | H | CF₂CF₃ | 261-263 |

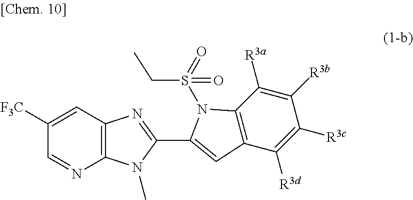

TABLE 2

| Compound No. | R^{3a} | R^{3b} | R^{3c} | R^{3d} | Physical property |
|---|---|---|---|---|---|
| 2-1 | H | H | H | H | 131-132 |
| 2-2 | Cl | H | H | H | |
| 2-3 | H | Cl | H | H | 170-172 |
| 2-4 | H | H | Cl | H | |
| 2-5 | H | H | H | Cl | 154-156 |
| 2-6 | Me | H | H | H | |
| 2-7 | H | Me | H | H | 104-107 |
| 2-8 | H | H | Me | H | |
| 2-9 | H | H | H | Me | |
| 2-10 | H | CF₃ | H | H | 124-128 |
| 2-11 | H | H | CF₃ | H | 134-135 |
| 2-12 | H | CN | H | H | |
| 2-13 | H | H | CN | H | |
| 2-14 | H | OMe | H | H | 171-173 |
| 2-15 | H | H | OMe | H | 208-212 |
| 2-16 | H | NH₂ | H | H | |
| 2-17 | H | H | NH₂ | H | |
| 2-18 | H | NMe₂ | H | H | |
| 2-19 | H | H | NMe₂ | H | |
| 2-20 | H | NHAc | H | H | |
| 2-21 | H | H | NHAc | H | |
| 2-22 | H | SMe | H | H | |
| 2-23 | H | H | SMe | H | |
| 2-24 | H | SCF₃ | H | H | |
| 2-25 | H | H | SCF₃ | H | |
| 2-26 | H | CHO | H | H | |
| 2-27 | H | CO₂Et | H | H | |
| 2-28 | H | CONHMe | H | H | |
| 2-29 | H | CH═NOEt | H | H | |
| 2-30 | H | c-Pr | H | H | 154-156 |
| 2-31 | H | Br | H | H | 175-177 |
| 2-32 | H | I | H | H | |
| 2-33 | H | H | H | CF₃ | |
| 2-34 | H | H | H | CN | |
| 2-35 | OMe | H | H | H | |
| 2-36 | H | H | H | OMe | |
| 2-37 | H | OCH₂CF₃ | H | H | |
| 2-38 | H | H | OCH₂CF₃ | H | |
| 2-39 | H | H | H | OCH₂CF₃ | |
| 2-40 | H | H | H | NH₂ | |
| 2-41 | H | H | H | NMe₂ | |
| 2-42 | H | H | H | NHAc | |
| 2-43 | H | H | H | SMe | |
| 2-44 | H | SOMe | H | H | |
| 2-45 | H | H | SOMe | H | |
| 2-46 | H | H | H | SOMe | |
| 2-47 | H | SO₂Me | H | H | |
| 2-48 | H | H | SO₂Me | H | |
| 2-49 | H | H | H | SO₂Me | |
| 2-50 | H | H | H | SCF₃ | |
| 2-51 | H | H | CHO | H | |
| 2-52 | H | H | H | CHO | |
| 2-53 | H | H | CO₂Et | H | |
| 2-54 | H | H | H | CO₂Et | |
| 2-55 | H | H | CONHMe | H | |
| 2-56 | H | H | H | CONHMe | |
| 2-57 | H | H | C═NOEt | H | |
| 2-58 | H | H | H | C═NOEt | |
| 2-59 | H | C═NOCH₂CF₃ | H | H | |
| 2-60 | H | H | C═NOCH₂CF₃ | H | |
| 2-61 | H | H | H | C═NOCH₂CF₃ | |
| 2-62 | H | H | c-Pr | H | 155-157 |
| 2-63 | H | H | H | c-Pr | |
| 2-64 | H | H | Br | H | 101-104 |
| 2-65 | H | H | OCF₃ | H | 113-115 |
| 2-66 | H | OEt | H | H | 127-128 |
| 2-67 | H | Cl | I | H | 204-205 |

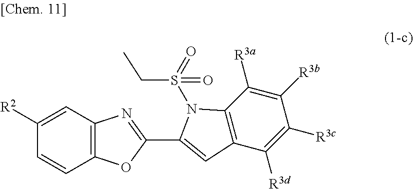

TABLE 3

| Compound | | | | | | Physical |
|---|---|---|---|---|---|---|
| No. | R^{3a} | R^{3b} | R^{3c} | R^{3d} | R² | property |
| 3-1 | H | H | H | H | SCF₃ | 118-119 |
| 3-2 | Cl | H | H | H | SCF₃ | |
| 3-3 | H | Cl | H | H | SCF₃ | |
| 3-4 | H | H | Cl | H | SCF₃ | 126-130 |
| 3-5 | H | H | H | Cl | SCF₃ | |
| 3-6 | Me | H | H | H | SCF₃ | |
| 3-7 | H | Me | H | H | SCF₃ | |
| 3-8 | H | H | Me | H | SCF₃ | |
| 3-9 | H | H | H | Me | SCF₃ | |
| 3-10 | H | CF₃ | H | H | SCF₃ | |
| 3-11 | H | H | CF₃ | H | SCF₃ | 155-157 |
| 3-12 | H | CN | H | H | SCF₃ | |
| 3-13 | H | H | CN | H | SCF₃ | |
| 3-14 | H | OMe | H | H | SCF₃ | |
| 3-15 | H | H | OMe | H | SCF₃ | |
| 3-16 | H | NH₂ | H | H | SCF₃ | |
| 3-17 | H | H | NH₂ | H | SCF₃ | |
| 3-18 | H | NMe₂ | H | H | SCF₃ | |
| 3-19 | H | H | NMe₂ | H | SCF₃ | |
| 3-20 | H | NHAc | H | H | SCF₃ | |
| 3-21 | H | H | NHAc | H | SCF₃ | |
| 3-22 | H | SMe | H | H | SCF₃ | |
| 3-23 | H | H | SMe | H | SCF₃ | |
| 3-24 | H | SCF₃ | H | H | SCF₃ | |
| 3-25 | H | H | SCF₃ | H | SCF₃ | |
| 3-26 | H | CHO | H | H | SCF₃ | |
| 3-27 | H | CO₂Et | H | H | SCF₃ | |
| 3-28 | H | CONHMe | H | H | SCF₃ | |
| 3-29 | H | CH═NOEt | H | H | SCF₃ | |
| 3-30 | H | c-Pr | H | H | SCF₃ | |
| 3-31 | H | Br | H | H | SCF₃ | |
| 3-32 | H | I | H | H | SCF₃ | |
| 3-33 | H | H | H | H | SO₂CF₃ | |
| 3-34 | Cl | H | H | H | SO₂CF₃ | |
| 3-35 | H | Cl | H | H | SO₂CF₃ | |
| 3-36 | H | H | Cl | H | SO₂CF₃ | |
| 3-37 | H | H | H | Cl | SO₂CF₃ | |
| 3-38 | Me | H | H | H | SO₂CF₃ | |
| 3-39 | H | Me | H | H | SO₂CF₃ | |
| 3-40 | H | H | Me | H | SO₂CF₃ | |
| 3-41 | H | H | H | Me | SO₂CF₃ | |
| 3-42 | H | CF₃ | H | H | SO₂CF₃ | |
| 3-43 | H | H | CF₃ | H | SO₂CF₃ | |
| 3-44 | H | CN | H | H | SO₂CF₃ | |
| 3-45 | H | H | CN | H | SO₂CF₃ | |
| 3-46 | H | OMe | H | H | SO₂CF₃ | |
| 3-47 | H | H | OMe | H | SO₂CF₃ | |
| 3-48 | H | NH₂ | H | H | SO₂CF₃ | |
| 3-49 | H | H | NH₂ | H | SO₂CF₃ | |
| 3-50 | H | NMe₂ | H | H | SO₂CF₃ | |
| 3-51 | H | H | NMe₂ | H | SO₂CF₃ | |
| 3-52 | H | NHAc | H | H | SO₂CF₃ | |
| 3-53 | H | H | NHAc | H | SO₂CF₃ | |
| 3-54 | H | SMe | H | H | SO₂CF₃ | |
| 3-55 | H | H | SMe | H | SO₂CF₃ | |
| 3-56 | H | SCF₃ | H | H | SO₂CF₃ | |
| 3-57 | H | H | SCF₃ | H | SO₂CF₃ | |
| 3-58 | H | CHO | H | H | SO₂CF₃ | |
| 3-59 | H | CO₂Et | H | H | SO₂CF₃ | |
| 3-60 | H | CONHMe | H | H | SO₂CF₃ | |
| 3-61 | H | C═NOEt | H | H | SO₂CF₃ | |
| 3-62 | H | c-Pr | H | H | SO₂CF₃ | |
| 3-63 | H | Br | H | H | SO₂CF₃ | |
| 3-64 | H | I | H | H | SO₂CF₃ | |

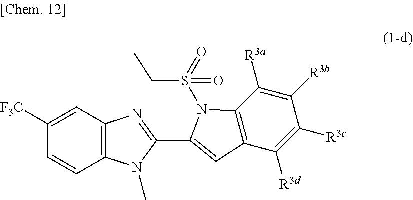

TABLE 4

| Compound No. | R^{3a} | R^{3b} | R^{3c} | R^{3d} | Physical property |
|---|---|---|---|---|---|
| 4-1 | H | H | H | H | 135-137 |
| 4-2 | Cl | H | H | H | |
| 4-3 | H | Cl | H | H | |
| 4-4 | H | H | Cl | H | |
| 4-5 | H | H | H | Cl | |
| 4-6 | Me | H | H | H | |
| 4-7 | H | Me | H | H | |
| 4-8 | H | H | Me | H | |
| 4-9 | H | H | H | Me | |
| 4-10 | H | CF₃ | H | H | |
| 4-11 | H | H | CF₃ | H | |
| 4-12 | H | CN | H | H | |
| 4-13 | H | H | CN | H | |
| 4-14 | H | OMe | H | H | |
| 4-15 | H | H | OMe | H | |
| 4-16 | H | NH₂ | H | H | |
| 4-17 | H | H | NH₂ | H | |
| 4-18 | H | NMe₂ | H | H | |
| 4-19 | H | H | NMe₂ | H | |
| 4-20 | H | NHAc | H | H | |
| 4-21 | H | H | NHAc | H | |
| 4-22 | H | SMe | H | H | |
| 4-23 | H | H | SMe | H | |
| 4-24 | H | SCF₃ | H | H | |
| 4-25 | H | H | SCF₃ | H | |
| 4-26 | H | CHO | H | H | |
| 4-27 | H | CO₂Et | H | H | |
| 4-28 | H | CONHMe | H | H | |
| 4-29 | H | CH═NOEt | H | H | |
| 4-30 | H | c-Pr | H | H | 106-108 |
| 4-31 | H | Br | H | H | 202-203 |
| 4-32 | H | I | H | H | |
| 4-33 | H | NHCO₂Et | H | H | |
| 4-34 | H | 4-F—Ph | H | H | 114-116 |

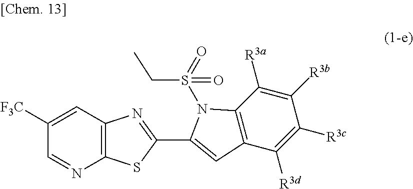

TABLE 5

| Compound No. | R^{3a} | R^{3b} | R^{3c} | R^{3d} | Physical property |
|---|---|---|---|---|---|
| 5-1 | H | H | H | H | |
| 5-2 | Cl | H | H | H | |
| 5-3 | H | Cl | H | H | |
| 5-4 | H | H | Cl | H | |
| 5-5 | H | H | H | Cl | |
| 5-6 | Me | H | H | H | |
| 5-7 | H | Me | H | H | |
| 5-8 | H | H | Me | H | |
| 5-9 | H | H | H | Me | |
| 5-10 | H | CF₃ | H | H | |
| 5-11 | H | H | CF₃ | H | |
| 5-12 | H | CN | H | H | |
| 5-13 | H | H | CN | H | |
| 5-14 | H | OMe | H | H | |
| 5-15 | H | H | OMe | H | |
| 5-16 | H | NH₂ | H | H | |
| 5-17 | H | H | NH₂ | H | |
| 5-18 | H | NMe₂ | H | H | |
| 5-19 | H | H | NMe₂ | H | |
| 5-20 | H | NHAc | H | H | |
| 5-21 | H | H | NHAc | H | |
| 5-22 | H | SMe | H | H | |
| 5-23 | H | H | SMe | H | |
| 5-24 | H | SCF₃ | H | H | |
| 5-25 | H | H | SCF₃ | H | |
| 5-26 | H | CHO | H | H | |
| 5-27 | H | CO₂Et | H | H | |
| 5-28 | H | CONHMe | H | H | |
| 5-29 | H | CH═NOEt | H | H | |
| 5-30 | H | c-Pr | H | H | |
| 5-31 | H | Br | H | H | |
| 5-32 | H | I | H | H | |

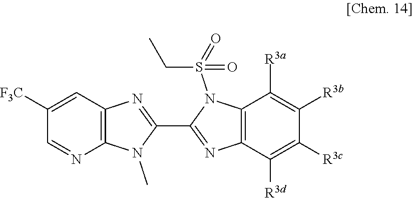

TABLE 6

| Compound No. | R^{3a} | R^{3b} | R^{3c} | R^{3d} | Physical property |
|---|---|---|---|---|---|
| 6-1 | H | H | H | H | NMR |
| 6-2 | H | Br | H | H | 203-206 |
| 6-3 | H | H | Br | H | 184-186 |
| 6-4 | H | Br | Br | H | |
| 6-5 | H | CF₃ | H | H | 172-173 |
| 6-6 | H | H | CF₃ | H | 147-148 |

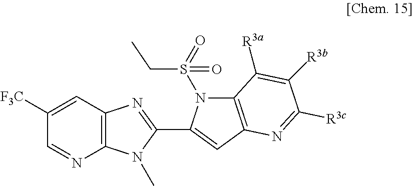

TABLE 7

| Compound No. | R^{3a} | R^{3b} | R^{3c} | Physical property |
|---|---|---|---|---|
| 7-1 | H | CF₃ | H | 159-163 |

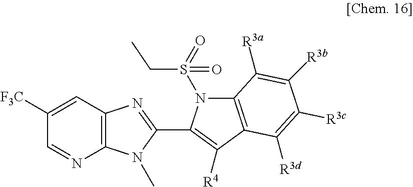

TABLE 8

| Compound | | | | | | Physical |
|---|---|---|---|---|---|---|
| No. | R^{3a} | R^{3b} | R^{3c} | R^{3d} | R⁴ | property |
| 8-1 | H | H | H | H | Me | |
| 8-2 | H | H | H | H | CN | |
| 8-3 | H | H | H | H | OMe | |
| 8-4 | H | H | H | H | OCH₂CF₃ | |
| 8-5 | H | H | H | H | NH₂ | |
| 8-6 | H | H | H | H | NMe₂ | |
| 8-7 | H | H | H | H | NHAc | |
| 8-8 | H | H | H | H | SMe | |
| 8-9 | H | H | H | H | SOMe | |
| 8-10 | H | H | H | H | SO₂Me | |
| 8-11 | H | H | H | H | SCF₃ | |
| 8-12 | H | H | H | H | CHO | |
| 8-13 | H | H | H | H | CO₂Et | |
| 8-14 | H | H | H | H | CONHMe | |
| 8-15 | H | H | H | H | C═NOEt | |
| 8-16 | H | H | H | H | C═NOCH₂CF₃ | |
| 8-17 | H | H | H | H | c-Pr | |
| 8-18 | H | H | Br | H | Cl | NMR |
| 8-19 | H | CI | Br | H | Br | 214-215 |
| 8-20 | H | H | H | Cl | Cl | 101-103 |
| 8-21 | H | CI | OMe | H | Cl | 190-193 |
| 8-22 | H | OMe | Cl | H | Cl | NMR |

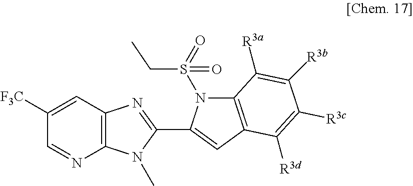

TABLE 9

| | | | | | |
|---|---|---|---|---|---|
| Compound No. | R^{3a} | R^{3b} | R^{3c} | R^{3d} | Physical property |

| | | | | |
|---|---|---|---|---|
| 9-1 | H | H | H | H |
| 9-2 | H | H | Cl | H |
| 9-3 | H | CF₃ | H | H |
| 9-4 | H | H | CF₃ | H |
| 9-5 | H | H | H | CF₃ |
| 9-6 | H | Br | H | H |

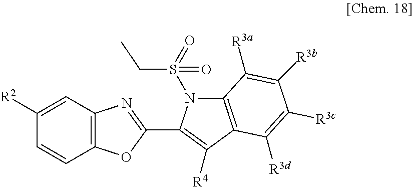

TABLE 10

| Compound | | | | | | Physical |
|---|---|---|---|---|---|---|
| No. | R^{3a} | R^{3b} | R^{3c} | R^{3d} | R⁴ | property |
| 10-1 | H | H | H | H | Me | |
| 10-2 | H | H | H | H | CN | |
| 10-3 | H | H | H | H | OMe | |
| 10-4 | H | H | H | H | OCH₂CF₃ | |
| 10-5 | H | H | H | H | NH₂ | |
| 10-6 | H | H | H | H | NMe₂ | |
| 10-7 | H | H | H | H | NHAc | |
| 10-8 | H | H | H | H | SMe | |
| 10-9 | H | H | H | H | SOMe | |
| 10-10 | H | H | H | H | SO₂Me | |
| 10-11 | H | H | H | H | SCF₃ | |
| 10-12 | H | H | H | H | CHO | |
| 10-13 | H | H | H | H | CO₂Et | |
| 10-14 | H | H | H | H | CONHMe | |
| 10-15 | H | H | H | H | C═NOEt | |
| 10-16 | H | H | H | H | C═NOCH₂CF₃ | |
| 10-17 | H | H | H | H | c-Pr | |
| 10-18 | H | H | Br | H | Cl | |
| 10-19 | H | Cl | Br | H | Br | |
| 10-20 | H | H | Cl | H | Cl | 112-114 |

TABLE 19

| Compound No. | ¹H-NMR data (CDCl₃) |
|---|---|
| 1-36 | δ 8.20 (s, 1H), 8.08 (d, 1H), 7.74 (d, 1H), 7.50 (dd, 1H), |
| | 7.12 (d, 1H), 4.33 (s, 1H), 4.11 (s, 1H), 3.56 (s, 3H), 1.27 (t, 3H) |
| 6-1 | δ 8.80 (s, 1H), 8.37 (s, 1H), 8.07 (d, 1H), 7.92 (d, 1H), |
| | 7.56-7.48 (m, 2H), 4.30 (q, 2H), 4.16 (s, 3H), 1.59 (t, 3H) |
| 8-18 | δ 8.80 (d, 1H), 8.36 (d, 1H), 8.03 (dd, 1H), 7.90 (d, 1H), |
| | 7.64 (dd, 1H), 3.89 (s, 3H), 3.90-3.80 (m, 1H), 3.58-3.47 (m, 1H), |
| | 1.36 (t, 3H) |
| 8-22 | δ 8.79 (d, 1H), 8.35 (d, 1H), 7.74 (s, 1H), 7.71 (s, 1H), 4.03 (s, |
| | 3H), 3.88 (s, 3H), 3.86-3.76 (m, 1H), 3.57-3.48 (m, 1H), |
| | 1.35 (t, 3H) |

The agricultural and horticultural insecticide comprising the compound represented by the general formula (1) of the present invention or a salt thereof as an active ingredient is suitable for controlling a variety of pests which may damage paddy rice, fruit trees, vegetables, other crops and ornamental flowering plants. The target pests are, for example, agricultural and forest pests, horticultural pests, stored grain pests, sanitary pests, nematodes, etc.

Specific examples of the pests, nematodes, etc. include the following:

the species of the order Lepidoptera such as Parasa consocia, Anomis mesogona, Papilio xuthus, Matsumuraeses azukivora, Ostrinia scapulalis, Spodoptera exempta, Hyphantria cunea, Ostrinia furnacalis, Pseudaletia separata, Tinea translucens, Bactra furfurana, Parnara guttata, Marasmia exigua, Parnara guttata, Sesamia inferens, Brachmia triannulella, Monema flavescens, Trichoplusia ni, Pleuroptya ruralis, Cystidia couaggaria, Lampides boeticus, Cephonodes hylas, Helicoverpa armigera, Phalerodonta manleyi, Eumeta japonica, Pieris brassicae, Malacosoma neustria testacea, Stathmopoda masinissa, Cuphodes diospyrosella, Archips xylosteanus, Agrotis segetum, Tetramoera schistaceana, Papilio machaon hippocrates, Endoclyta sinensis, Lyonetia prunifoliella, Phyllonorycter ringoneella, Cydia kurokoi, Eucoenogenes aestuosa, Lobesia botrana, Latoia sinica, Euzophera batangensis, Phalonidia mesotypa, Spilosoma imparilis, Glyphodes pyloalis, Olethreutes mori, Tineola bisselliella, Endoclyta excrescens, Nemapogon granellus, Synanthedon hector, Cydia pomonella, Plutella xylostella, Cnaphalocrocis medinalis, Sesamia calamistis, Scirpophaga incertulas, Pediasia teterrellus, Phthorimaea operculella, Stauropus fagi persimilis, Etiella zinckenella, Spodoptera exigua, Palpifer sexnotata, Spodoptera mauritia, Scirpophaga innotata, Xestia c-nigrum, Spodoptera depravata, Ephestia kuehniella, Angerona prunaria, Clostera anastomosis, Pseudoplusia includens, Matsumuraeses falcana, Helicoverpa assulta, Autographa nigrisigna, Agrotis Euproctis pseudoconspersa, Adoxophyes orana, Caloptilia theivora, Homona magnanima, Ephestia elutella, Eumeta minuscula, Clostera anachoreta, Heliothis maritima, Sparganothis pilleriana, Busseola fusca, Euproctis subflava, Biston robustum, Heliothis zea, Aedia leucomelas, Narosoideus flavidorsalis, Viminia rumicis, Bucculatrix pyrivorella, Grapholita molesta, Spulerina astaurota, Ectomyelois pyrivorella, Chilo suppressalis, Acrolepiopsis sapporensis, Plodia interpunctella, Hellula undalis, Sitotroga cerealella, Spodoptera litura, a species of the family Tortricidae (Eucosma aporema), Acleris comariana, Scopelodes contractus, Orgyia thyellina, Spodoptera frugiperda, Ostrinia zaguliaevi, Naranga aenescens, Andraca bipunctata, Paranthrene regalis, Acosmeryx castanea, Phyllocnistis toparcha, Endopiza viteana, Eupoecillia ambiguella, Anticarsia gemmatalis, Cnephasia cinereipalpana, Lymantria dispar, Dendrolimus spectabilis, Leguminivora glycinivorella, Maruca testulalis, Matsumuraeses phaseoli, Caloptilia soyella, Phyllocnistis citrella, Omiodes indicata, Archips fuscocupreanus, Acanthoplusia agnata, Bambalina sp., Carposina niponensis, Conogethes punctiferalis, Synanthedon sp., Lyonetia clerkella, Papilio helenus, Colias erate poliographus, Phalera flavescens, the species of the family Pieridae such as Pieris rapae crucivora and Pieris rapae, Euproctis similis, Acrolepiopsis suzukiella, Ostrinia nubilalis, Mamestra brassicae, Ascotis selenaria, Phtheochroides clandestina, Hoshinoa adumbratana, Odonestis pruni japonensis, Triaena intermedia, Adoxophyes orana fasciata, Grapholita inopinata, Spilonota ocellana, Spilonota lechriaspis, Illiberis pruni, Argyresthia conjugella, Caloptilia zachrysa, Archips breviplicanus, Anomis flava, Pectinophora gossypiella, Notarcha derogata, Diaphania indica, Heliothis virescens and Earias cupreoviridis;

the species of the order Hemiptera such as Nezara antennata, Stenotus rubrovittatus, Graphosoma rubrolineatum, Trigonotylus coelestialium, Aeschynteles maculatus, Creontiades pallidifer, Dysdercus cingulatus, Chrysomphalus ficus, Aonidiella aurantii, Graptopsaltria nigrofuscata, Blissus leucopterus, Icerya purchasi, Piezodorus hybneri, Lagynotomus elongatus, Thaia subrufa, Scotinophara lurida, Sitobion ibarae, Stariodes iwasakii, Aspidiotus destructor, Taylorilygus pallidulus, Myzus mumecola, Pseudaulacaspis prunicola, Acyrthosiphon pisum, Anacanthocoris striicornis, Ectometopterus micantulus, Eysarcoris lewisi, Molipteryx fuliginosa, Cicadella viridis, Rhopalosophum rufiabdominalis, Saissetia oleae, Trialeurodes vaporariorum, Aguriahana quercus, Lygus spp., Euceraphis punctipennis, Andaspis kashicola, Coccus pseudomagnoliarum, Cavelerius saccharivorus, Galeatus spinifrons, Macrosiphoniella sanborni, Aonidiella citrina, Halyomorpha mista, Stephanitis fasciicarina, Trioza camphorae, Leptocorisa chinensis, Trioza quercicola, Uhlerites latius, Erythroneura comes, Paromius exiguus, Duplaspidiotus claviger, Nephotettix nigropictus, Halticiellus insularis, Perkinsiella saccharicida, Psylla malivorella, Anomomeura mori, Pseudococcus longispinis, Pseudaulacaspis pentagona, Pulvinaria kuwacola, Apolygus lucorum, Togo hemipterus, Toxoptera aurantii, Saccharicoccus sacchari, Geoica lucifuga, Numata muiri, Comstockaspis perniciosa, Unaspis citri, Aulacorthum solani, Eysarcoris ventralis, Bemisia argentifolii, Cicadella spectra, Aspidiotus hederae, Liorhyssus hyalinus, Calophya nigridorsalis, Sogatella furcifera, Megoura crassicauda, Brevicoryne brassicae, Aphis glycines, Leptocorisa oratorios, Nephotettix virescens, Uroeucon formosanum, Cyrtopeltis tennuis, Bemisia tabaci, Lecanium persicae, Parlatoria theae, Pseudaonidia paeoniae, Empoasca onukii, Plautia stali, Dysaphis tulipae, Macrosiphum euphorbiae, Stephanitis pyrioides, Ceroplastes ceriferus, Parlatoria camelliae, Apolygus spinolai, Nephotettix cincticeps, Glaucias subpunctatus, Orthotylus flavosparsus, Rhopalosiphum maidis, Peregrinus maidis, Eysarcoris parvus, Cimex lectularius, Psylla abieti, Nilaparvata lugens, Psylla tobirae, Eurydema rugosum, Schizaphis piricola, Psylla pyricola, Parlatoreopsis pyri, Stephanitis nashi, Dysmicoccus wistariae, Lepholeucaspis japonica, Sappaphis piri, Lipaphis erysimi, Neotoxoptera formosana, Rhopalosophum nymphaeae, Edwardsiana rosae, Pinnaspis aspidistrae, Psylla alni, Speusotettix subfusculus, Alnetoidia alneti, Sogatella panicicola, Adelphocoris lineolatus, Dysdercus poecilus, Parlatoria ziziphi, Uhlerites debile, Laodelphax striatellus, Eurydema pulchrum, Cletus trigones, Clovia punctata, Empoasca spp., Coccus hesperidum, Pachybrachius luridus, Planococcus kraunhiae, Stenotus binotatus, Arboridia apicalis, Macrosteles fascifrons, Dolycoris baccarum, Adelphocoris triannulatus, Viteus vitifolii, Acanthocoris sordidus, Leptocorisa aceta, Macropes obnubilus, Cletus punctiger, Riptortus clavatus, Paratrioza cockerelli, Aphrophora costalis, Lygus disponsi, Lygus saundersi, Crisicoccus pini, Empoasca abietis, Crisicoccus matsumotoi, Aphis craccivora, Megacopta punctatissimum, Eysarcoris guttiger, Lepidosaphes beckii, Diaphorina citri, Toxoptera citricidus, Planococcus citri, Dialeurodes citri, Aleurocanthus spiniferus, Pseudococcus citriculus, Zyginella citri, Pulvinaria citricola, Coccus discrepans, Pseudaonidia duplex, Pulvinaria aurantii, Lecanium corni, Nezara viridula, Stenodema calcaratum, Rhopalosiphum padi, Sitobion akebiae, Schizaphis graminum, Sorhoanus tritici, Brachycaudus helichrysi, Carpocoris purpureipennis, Myzus persicae, Hyalopterus pruni, Aphis farinose yanagicola, Metasalis populi, Unaspis yanonensis, Mesohomotoma camphorae, Aphis spiraecola, Aphis pomi, Lepidosaphes ulmi, Psylla mali, Heterocordylus flavipes, Myzus malisuctus, Aphidonuguis mali, Orientus ishidai, Ovatus malicolens, Eriosoma lanigerum, Ceroplastes rubens and Aphis gossypii;

the species of the order Coleoptera such as Xystrocera globosa, Paederus fuscipes, Eucetonia roelofsi, Callosobruchus chinensis, Cylas formicarius, Hypera postica, Echinocnemus squameus, Oulema oryzae, Donacia provosti, Lissorhoptrus oryzophilus, Colasposoma dauricum, Euscepes postfasciatus, Epilachna varivestis, Acanthoscelides obtectus, Diabrotica virgifera virgifera, Involvulus cupreus, Aulacophora femoralis, Bruchus pisorum, Epilachna vigintioctomaculata, Carpophilus dimidiatus, Cassida nebulosa, Luperomorpha tunebrosa, Phyllotreta striolata, Psacothea hilaris, Aeolesthes chrysothrix, Curculio sikkimensis, Carpophilus hemipterus, Oxycetonia jucunda, Diabrotica spp., Mimela splendens, Sitophilus zeamais, Tribolium castaneum, Sitophilus oryzae, Palorus subdepressus, Melolontha japonica, Anoplophora malasiaca, Meatus picipes, Leptinotarsa decemlineata, Diabrotica undecimpunctata howardi, Sphenophorus venatus, Crioceris quatuordecimpunctata, Conotrachelus nenuphar, Ceuthorhynchidius albosuturalis, Phaedon brassicae, Lasioderma serricorne, Sitona japonicus, Adoretus tenuimaculatus, Tenebrio molitor, Basilepta balyi, Hypera nigrirostris, Chaetocnema concinna, Anomala cuprea, Heptophylla picea, Epilachna vigintioctopunctata, Diabrotica longicornis, Eucetonia pilifera, Agriotes spp., Attagenus unicolor japonicus, Pagria signata, Anomala rufocuprea, Palorus ratzeburgii, Alphitobius laevigatus, Anthrenus verbasci, Lyctus brunneus, Tribolium confusum, Medythia nigrobilineata, Xylotrechus pyrrhoderus, Epitrix cucumeris, Tomicus piniperda, Monochamus alternatus, Popillia japonica, Epicauta gorhami, Sitophilus zeamais, Rhynchites heros, Listroderes costirostris, Callosobruchus maculatus, Phyllobius armatus, Anthonomus pomorum, Linaeidea aenea and Anthonomus grandis;

the species of the order Diptera such as Culex pipiens pallens, Pegomya hyoscyami, Liriomyza huidobrensis, Musca domestica, Chlorops oryzae, Hydrellia sasakii, Agromyza oryzae, Hydrellia griseola, Hydrellia griseola, Ophiomyia phaseoli, Dacus cucurbitae, Drosophila suzukii, Rhacochlaena japonica, Muscina stabulans, the species of the family Phoridae such as Megaselia spiracularis, Clogmia albipunctata, Tipula aino, Phormia regina, Culex tritaeniorhynchus, Anopheles sinensis, Hylemya brassicae, Asphondylia sp., Delia platura, Delia antiqua, Rhagoletis cerasi, Culex pipiens molestus Forskal, Ceratitis capitata, Bradysia agrestis, Pegomya cunicularia, Liriomyza sativae, Liriomyza bryoniae, Chromatomyia horticola, Liriomyza chinensis, Culex quinquefasciatus, Aedes aegypti, Aedes albopictus, Liriomyza trifolii, Liriomyza sativae, Dacus dorsalis, Dacus tsuneonis, Sitodiplosis mosellana, Meromuza nigriventris, Anastrepha ludens and Rhagoletis pomonella;

the species of the order Hymenoptera such as Pristomyrmex pungens, the species of the family Bethylidae, Monomorium pharaonis, Pheidole noda, Athalia rosae, Dryocosmus kuriphilus, Formica fusca japonica, the species of the subfamily Vespinae, Athalia infumata infumata, Arge pagana, Athalia japonica, Acromyrmex spp., Solenopsis spp., Arge mali and Ochetellus glaber;

the species of the order Orthoptera such as Homorocoryphus lineosus, Gryllotalpa sp., Oxya hyla intricata, Oxya yezoensis, Locusta migratoria, Oxya japonica, Homorocoryphus jezoensis and Teleogryllus emma;

the species of the order Thysanoptera such as Selenothrips rubrocinctus, Stenchaetothrips biformis, Haplothrips aculeatus, Ponticulothrips diospyrosi, Thrips flavus, Anaphothrips obscurus, Liothrips floridensis, Thrips simplex, Thrips nigropilosus, Heliothrips haemorrhoidalis, Pseudodendrothrips mori, Microcephalothrips abdominalis, Leeuwenia pasanii, Litotetothrips pasaniae, Scirtothrips citri, Haplothrips chinensis, Mycterothrips glycines, Thrips setosus, Scirtothrips dorsalis, Dendrothrips minowai, Haplothrips niger, Thrips tabaci, Thrips alliorum, Thrips hawaiiensis, Haplothrips kurdjumovi, Chirothrips manicatus, Frankliniella intonsa, Thrips coloratus, Franklinella occidentalis, Thrips palmi, Frankliniella lilivora and Liothrips vaneeckei;

the species of the order Acari such as Leptotrombidium akamushi, Tetranychus ludeni, Dermacentor variabilis, Tetranychus truncatus, Ornithonyssus bacoti, Demodex canis, Tetranychus viennensis, Tetranychus kanzawai, the species of the family Ixodidae such as Rhipicephalus sanguineus, Cheyletus malaccensis, Tyrophagus putrescentiae, Dermatophagoides farinae, Latrodectus hasseltii, Dermacentor taiwanicus, Acaphylla theavagrans, Polyphagotarsonemus latus, Aculops lycopersici, Ornithonyssus sylvairum, Tetranychus urticae, Eriophyes chibaensis, Sarcoptes scabiei, Haemaphysalis longicornis, Ixodes scapularis, Tyrophagus similis, Cheyletus eruditus, Panonychus citri, Cheyletus moorei, Brevipalpus phoenicis, Octodectes cynotis, Dermatophagoides ptrenyssnus, Haemaphysalis flava, Ixodes ovatus, Phyllocoptruta citri, Aculus schlechtendali, Panonychus ulmi, Amblyomma americanum, Dermanyssus gallinae, Rhyzoglyphus robini and Sancassania sp.;

the species of the order Isoptera such as Reticulitermes miyatakei, Incisitermes minor, Coptotermes formosanus, Hodotermopsis japonica, Reticulitermes sp., Reticulitermes flaviceps amamianus, Glyptotermes kushimensis, Coptotermes guangzhoensis, Neotermes koshunensis, Glyptotermes kodamai, Glyptotermes satsumensis, Cryptotermes domesticus, Odontotermes formosanus, Glyptotermes nakajimai, Pericapritermes nitobei and Reticulitermes speratus;

the species of the order Blattodea such as Periplaneta fuliginosa, Blattella germanica, Blatta orientalis, Periplaneta brunnea, Blattella lituricollis, Periplaneta japonica and Periplaneta americana;

the species of the order Siphonaptera such as Pulex irritans, Ctenocephalides felis and Ceratophyllus gallinae;

the species of the phylum Nematoda such as Nothotylenchus acris, Aphelenchoides besseyi, Pratylenchus penetrans, Meloidogyne hapla, Meloidogyne incognita, Globodera rostochiensis, Meloidogyne javanica, Heterodera glycines, Pratylenchus coffeae, Pratylenchus neglectus and Tylenchus semipenetrans; and

the species of the phylum Mollusca such as Pomacea canaliculata, Achatina fulica, Meghimatium bilineatum, Lehmannina valentiana, Limax flavus and Acusta despecta sieboldiana.

In addition, the agricultural and horticultural insecticide of the present invention has a strong insecticidal effect on Tuta absoluta as well.

Further, mites and ticks parasitic on animals are also included in the target pests, and the examples include the species of the family Ixodidae such as Boophilus microplus, Rhipicephalus sanguineus, Haemaphysalis longicornis, Haemaphysalis flava, Haemaphysalis campanulata, Haemaphysalis concinna, Haemaphysalis japonica, Haemaphysalis kitaokai, Haemaphysalis ias, Ixodes ovatus, Ixodes nipponensis, Ixodes persulcatus, Amblyomma testudinarium, Haemaphysalis megaspinosa, Dermacentor reticulatus and Dermacentor taiwanesis; Dermanyssus gallinae; the species of the genus Ornithonyssus such as Ornithonyssus sylviarum and Ornithonyssus bursa; the species of the family Trombiculidae such as Eutrombicula wichmanni, Leptotrombidium akamushi, Leptotrombidium pallidum, Leptotrombidium fuji, Leptotrombidium Cosa, Neotrombicula autumnalis, Eutrombicula alfreddugesi and Helenicula miyagawai; the species of the family Cheyletidae such as Cheyletiella yasguri, Cheyletiella parasitivorax and Cheyletiella blakei; the species of the superfamily Sarcoptoidea such as Psoroptes cuniculi, Chorioptes bovis, Otodectes cynotis, Sarcoptes scabiei and Notoedres cati; and the species of the family Demodicidae such as Demodex canis.

Other target pests include fleas including ectoparasitic wingless insects belonging to the order Siphonaptera, more specifically, the species belonging to the families Pulicidae and Ceratophyllidae. Examples of the species belonging to the family Pulicidae include Ctenocephalides canis, Ctenocephalides felis, Pulex irritans, Echidnophaga gallinacea, Xenopsylla cheopis, Leptopsylla segnis, Nosopsyllus fasciatus and Monopsyllus anisus.

Other target pests include ectoparasites, for example, the species of the suborder Anoplura such as Haematopinus eurysternus, Haematopinus asini, Dalmalinia ovis, Linognathus vituli, Haematopinus suis, Phthirus pubis and Pediculus capitis; the species of the suborder Mallophaga such as Trichodectes canis; and hematophagous Dipteran insect pests such as Tabanus trigonus, Culicoides schultzei and Simulium ornatum. In addition, examples of endoparasites include nematodes such as lungworms, whipworms, nodular worms, endogastric parasitic worms, ascarides and filarial worms; cestodes such as Spirometra erinacei, Diphyllobothrium latum, Dipylidium caninum, Multiceps multiceps, Echinococcus granulosus and Echinococcus multilocularis; trematodes such as Schistosoma japonicum and Fasciola hepatica; and protozoa such as coccidia, Plasmodium, intestinal Sarcocystis, Toxoplasma and Cryptosporidium.

The agricultural and horticultural insecticide comprising the compound represented by the general formula (1) of the present invention or a salt thereof as an active ingredient has a remarkable control effect on the above-described pests which damage lowland crops, field crops, fruit trees, vegetables, other crops, ornamental flowering plants, etc. The desired effect can be obtained when the agricultural and horticultural insecticide is applied to nursery facilities for seedlings, paddy fields, fields, fruit trees, vegetables, other crops, ornamental flowering plants, etc. and their seeds, paddy water, foliage, cultivation media such as soil, or the like around the expected time of pest infestation, i.e., before the infestation or upon the confirmation of the infestation. In particularly preferable embodiments, the application of the agricultural and horticultural insecticide utilizes so-called penetration and translocation. That is, nursery soil, soil in transplanting holes, plant foot, irrigation water, cultivation water in hydroponics, or the like is treated with the agricultural and horticultural insecticide to allow crops, ornamental flowering plants, etc. to absorb the compound of the present invention through the roots via soil or otherwise.

Examples of useful plants to which the agricultural and horticultural insecticide of the present invention can be applied include, but are not particularly limited to, cereals (e.g., rice, barley, wheat, rye, oats, corn, etc.), legumes (e.g., soybeans, azuki beans, broad beans, green peas, kidney beans, peanuts, etc.), fruit trees and fruits (e.g., apples, citrus fruits, pears, grapes, peaches, plums, cherries, walnuts, chestnuts, almonds, bananas, etc.), leaf and fruit vegetables (e.g., cabbages, tomatoes, spinach, broccoli, lettuce, onions, green onions (chives and Welsh onions), green peppers, eggplants, strawberries, pepper crops, okra, Chinese chives, etc.), root vegetables (e.g., carrots, potatoes, sweet potatoes, taros, Japanese radishes, turnips, lotus roots, burdock roots, garlic, Chinese scallions, etc.), crops for processing (e.g., cotton, hemp, beet, hops, sugarcane, sugar beet, olives, rubber, coffee, tobacco, tea, etc.), gourds (e.g., Japanese pumpkins, cucumbers, watermelons, oriental sweet melons, melons, etc.), pasture grass (e.g., orchardgrass, sorghum, timothy, clover, alfalfa, etc.), lawn grass (e.g., Korean lawn grass, bent grass, etc.), spice and aromatic crops and ornamental crops (e.g., lavender, rosemary, thyme, parsley, pepper, ginger, etc.), ornamental flowering plants (e.g., chrysanthemum, rose, carnation, orchid, tulip, lily, etc.), garden trees (e.g., ginkgo trees, cherry trees, Japanese aucuba, etc.) and forest trees (e.g., Abies sachalinensis, Picea jezoensis, pine, yellow cedar, Japanese cedar, hinoki cypress, eucalyptus, etc.).

The above-mentioned “plants” also include plants provided with herbicide tolerance by a classical breeding technique or a gene recombination technique. Examples of such herbicide tolerance include tolerance to HPPD inhibitors, such as isoxaflutole; ALS inhibitors, such as imazethapyr and thifensulfuron-methyl; EPSP synthase inhibitors, such as glyphosate; glutamine synthetase inhibitors, such as glufosinate; acetyl-CoA carboxylase inhibitors, such as sethoxydim; or other herbicides, such as bromoxynil, dicamba and 2,4-D.

Examples of the plants provided with herbicide tolerance by a classical breeding technique include varieties of rapeseed, wheat, sunflower and rice tolerant to the imidazolinone family of ALS-inhibiting herbicides such as imazethapyr, and such plants are sold under the trade name of Clearfield (registered trademark). Also included is a variety of soybean provided with tolerance to the sulfonyl urea family of ALS-inhibiting herbicides such as thifensulfuron-methyl by a classical breeding technique, and this is sold under the trade name of STS soybean. Also included are plants provided with tolerance to acetyl-CoA carboxylase inhibitors such as trione oxime herbicides and aryloxy phenoxy propionic acid herbicides by a classical breeding technique, for example, SR corn and the like.

Plants provided with tolerance to acetyl-CoA carboxylase inhibitors are described in Proc. Natl. Acad. Sci. USA, 87, 7175-7179 (1990), and the like. Further, acetyl-CoA carboxylase mutants resistant to acetyl-CoA carboxylase inhibitors are reported in Weed Science, 53, 728-746 (2005), and the like, and by introducing the gene of such an acetyl-CoA carboxylase mutant into plants by a gene recombination technique, or introducing a resistance-conferring mutation into acetyl-CoA carboxylase of plants, plants tolerant to acetyl-CoA carboxylase inhibitors can be engineered. Alternatively, by introducing a nucleic acid causing base substitution mutation into plant cells (a typical example of this technique is chimeraplasty technique (Gura T. 1999. Repairing the Genome's Spelling Mistakes. Science 285: 316-318.)) to allow site-specific substitution mutation in the amino acids encoded by an acetyl-CoA carboxylase gene, an ALS gene or the like of plants, plants tolerant to acetyl-CoA carboxylase inhibitors, ALS inhibitors or the like can be engineered. The agricultural and horticultural insecticide of the present invention can be applied to these plants as well.

Further, exemplary toxins expressed in genetically modified plants include insecticidal proteins of Bacillus cereus or Bacillus popilliae; Bacillus thuringiensis δ-endotoxins, such as Cry1Ab, Cry1Ac, Cry1F, Cry1Fa2, Cry2Ab, Cry3A, Cry3Bb1 and Cry9C, and other insecticidal proteins, such as VIP1, VIP2, VIP3 and VIP3A; nematode insecticidal proteins; toxins produced by animals, such as scorpion toxins, spider toxins, bee toxins and insect-specific neurotoxins; toxins of filamentous fungi; plant lectins; agglutinin; protease inhibitors, such as trypsin inhibitors, serine protease inhibitors, patatin, cystatin and papain inhibitors; ribosome inactivating proteins (RIP), such as ricin, maize RIP, abrin, luffin, saporin and bryodin; steroid metabolizing enzymes, such as 3-hydroxy steroid oxidase, ecdysteroid-UDP-glucosyltransferase and cholesterol oxidase; ecdysone inhibitors; HMG-CoA reductase; ion channel inhibitors, such as sodium channel inhibitors and calcium channel inhibitors; juvenile hormone esterase; diuretic hormone receptors; stilbene synthase; bibenzyl synthase; chitinase; and glucanase.

Also included are hybrid toxins, partially deficient toxins and modified toxins derived from the following: δ-endotoxin proteins such as Cry1Ab, Cry1Ac, Cry1F, Cry1Fa2, Cry2Ab, Cry3A, Cry3Bb1, Cry9C, Cry34Ab and Cry35Ab, and other insecticidal proteins such as VIP1, VIP2, VIP3 and VIP3A. The hybrid toxin can be produced by combining some domains of these proteins differently from the original combination in nature with the use of a recombination technique. As the partially deficient toxin, a Cry1Ab toxin in which a part of the amino acid sequence is deleted is known. In the modified toxin, one or more amino acids of a naturally occurring toxin are substituted.

Examples of the foregoing toxins and genetically modified plants capable of synthesizing these toxins are described in EP-A-0 374 753, WO 93/07278, WO 95/34656, EP-A-0 427 529, EP-A-451 878, WO 03/052073, etc.

Due to the toxins contained in such genetically modified plants, the plants exhibit resistance to pests, in particular, Coleopteran insect pests, Hemipteran insect pests, Dipteran insect pests, Lepidopteran insect pests and nematodes. The above-described technologies and the agricultural and horticultural insecticide of the present invention can be used in combination or used systematically.

In order to control target pests, the agricultural and horticultural insecticide of the present invention, with or without appropriate dilution or suspension in water etc., is applied to plants potentially infested with the target insect pests or nematodes in an amount effective for the control of the insect pests or nematodes. For example, in order to control insect pests and nematodes that may damage crop plants such as fruit trees, cereals and vegetables, foliar application and seed treatment such as dipping, dust coating and calcium peroxide coating can be performed. Further, treatment of soil or the like may also be performed to allow plants to absorb agrochemicals through their roots. Examples of such treatment include whole soil incorporation, planting row treatment, bed soil incorporation, plug seedling treatment, planting hole treatment, plant foot treatment, top-dressing, treatment of nursery boxes for paddy rice, and submerged application. In addition, application to culture media in hydroponics, smoking treatment, trunk injection and the like can also be performed.

Further, the agricultural and horticultural insecticide of the present invention, with or without appropriate dilution or suspension in water etc., can be applied to sites potentially infested with pests in an amount effective for the control of the pests. For example, it can be directly applied to stored grain pests, house pests, sanitary pests, forest pests, etc., and also be used for coating of residential building materials, for smoking treatment, or as a bait formulation.

Exemplary methods of seed treatment include dipping of seeds in a diluted or undiluted fluid of a liquid or solid formulation for the permeation of agrochemicals into the seeds; mixing or dust coating of seeds with a solid or liquid formulation for the adherence of the formulation onto the surfaces of the seeds; coating of seeds with a mixture of a solid or liquid formulation and an adhesive carrier such as resins and polymers; and application of a solid or liquid formulation to the vicinity of seeds at the same time as seeding.

The term “seed” in the above-mentioned seed treatment refers to a plant body which is in the early stages of cultivation and used for plant propagation. The examples include, in addition to a so-called seed, a plant body for vegetative propagation, such as a bulb, a tuber, a seed potato, a bulbil, a propagule, a discoid stem and a stem used for cuttage.

The term “soil” or “cultivation medium” in the method of the present invention for using an insecticide refers to a support medium for crop cultivation, in particular a support medium which allows crop plants to spread their roots therein, and the materials are not particularly limited as long as they allow plants to grow. Examples of the support medium include what is called soils, seedling mats and water, and specific examples of the materials include sand, pumice, vermiculite, diatomite, agar, gelatinous substances, high-molecular-weight substances, rock wool, glass wool, wood chip and bark.

Exemplary methods of the application to crop foliage or to stored grain pests, house pests, sanitary pests, forest pests, etc. include application of a liquid formulation, such as an emulsifiable concentrate and a flowable, or a solid formulation, such as a wettable powder and a water-dispersible granule, after appropriate dilution in water; dust application; and smoking.

Exemplary methods of soil application include application of a water-diluted or undiluted liquid formulation to the foot of plants, nursery beds for seedlings, or the like; application of a granule to the foot of plants, nursery beds for seedlings, or the like; application of a dust, a wettable powder, a water-dispersible granule, a granule or the like onto soil and subsequent incorporation of the formulation into the whole soil before seeding or transplanting; and application of a dust, a wettable powder, a water-dispersible granule, a granule or the like to planting holes, planting rows or the like before seeding or planting.

To nursery boxes for paddy rice, for example, a dust, a water-dispersible granule, a granule or the like can be applied, although the suitable formulation may vary depending on the application timing, in other words, depending on the cultivation stage such as seeding time, greening period and planting time. A formulation such as a dust, a water-dispersible granule and a granule may be mixed with nursery soil. For example, such a formulation is incorporated into bed soil, covering soil or the whole soil. Simply, nursery soil and such a formulation may be alternately layered.

In the application to paddy fields, a solid formulation, such as a jumbo, a pack, a granule and a water-dispersible granule, or a liquid formulation, such as a flowable and an emulsifiable concentrate, is applied usually to flooded paddy fields. In a rice planting period, a suitable formulation, as it is or after mixed with a fertilizer, may be applied onto soil or injected into soil. In addition, an emulsifiable concentrate, a flowable or the like may be applied to the source of water supply for paddy fields, such as a water inlet and an irrigation device. In this case, treatment can be accomplished with the supply of water and thus achieved in a labor-saving manner.

In the case of field crops, their seeds, cultivation media in the vicinity of their plants, or the like may be treated in the period of seeding to seedling culture. In the case of plants of which the seeds are directly sown in the field, in addition to direct seed treatment, plant foot treatment during cultivation is preferable. Specifically, the treatment can be performed by, for example, applying a granule onto soil, or drenching soil with a formulation in a water-diluted or undiluted liquid form. Another preferable treatment is incorporation of a granule into cultivation media before seeding.

In the case of culture plants to be transplanted, preferable examples of the treatment in the period of seeding to seedling culture include, in addition to direct seed treatment, drench treatment of nursery beds for seedlings with a formulation in a liquid form; and granule application to nursery beds for seedlings. Also included are treatment of planting holes with a granule; and incorporation of a granule into cultivation media in the vicinity of planting points at the time of fix planting.

The agricultural and horticultural insecticide of the present invention is commonly used as a formulation convenient for application, which is prepared in the usual method for preparing agrochemical formulations.

That is, the condensed heterocyclic compound represented by the general formula (1) of the present invention or a salt thereof and an appropriate inactive carrier, and if needed an adjuvant, are blended in an appropriate ratio, and through the step of dissolution, separation, suspension, mixing, impregnation, adsorption and/or adhesion, are formulated into an appropriate form for application, such as a suspension concentrate, an emulsifiable concentrate, a soluble concentrate, a wettable powder, a water-dispersible granule, a granule, a dust, a tablet and a pack.

The composition (agricultural and horticultural insecticide or animal parasite control agent) of the present invention can optionally contain an additive usually used for agrochemical formulations or animal parasite control agents in addition to the active ingredient. Examples of the additive include carriers such as solid or liquid carriers, surfactants, dispersants, wetting agents, binders, tackifiers, thickeners, colorants, spreaders, sticking/spreading agents, antifreezing agents, anti-caking agents, disintegrants and stabilizing agents. If needed, preservatives, plant fragments, etc. may also be used as the additive. One of these additives may be used alone, and also two or more of them may be used in combination.

Examples of the solid carriers include natural minerals, such as quartz, clay, kaolinite, pyrophyllite, sericite, talc, bentonite, acid clay, attapulgite, zeolite and diatomite; inorganic salts, such as calcium carbonate, ammonium sulfate, sodium sulfate and potassium chloride; organic solid carriers, such as synthetic silicic acid, synthetic silicates, starch, cellulose and plant powders (for example, sawdust, coconut shell, corn cob, tobacco stalk, etc.); plastics carriers, such as polyethylene, polypropylene and polyvinylidene chloride; urea; hollow inorganic materials; hollow plastic materials; and fumed silica (white carbon). One of these solid carriers may be used alone, and also two or more of them may be used in combination.

Examples of the liquid carriers include alcohols including monohydric alcohols, such as methanol, ethanol, propanol, isopropanol and butanol, and polyhydric alcohols, such as ethylene glycol, diethylene glycol, propylene glycol, hexylene glycol, polyethylene glycol, polypropylene glycol and glycerin; polyol compounds, such as propylene glycol ether; ketones, such as acetone, methyl ethyl ketone, methyl isobutyl ketone, diisobutyl ketone and cyclohexanone; ethers, such as ethyl ether, dioxane, ethylene glycol monoethyl ether, dipropyl ether and tetrahydrofuran (THF); aliphatic hydrocarbons, such as normal paraffin, naphthene, isoparaffin, kerosene and mineral oil; aromatic hydrocarbons, such as benzene, toluene, xylene, solvent naphtha and alkyl naphthalene; halogenated hydrocarbons, such as dichloromethane, chloroform and carbon tetrachloride; esters, such as ethyl acetate, diisopropyl phthalate, dibutyl phthalate, dioctyl phthalate and dimethyl adipate; lactones, such as γ-butyrolactone; amides, such as dimethylformamide, diethylformamide, dimethylacetamide and N-alkyl pyrrolidinone; nitriles, such as acetonitrile; sulfur compounds, such as dimethyl sulfoxide; vegetable oils, such as soybean oil, rapeseed oil, cotton seed oil and castor oil; and water. One of these liquid carriers may be used alone, and also two or more of them may be used in combination.

Exemplary surfactants used as the dispersant or the wetting/spreading agent include nonionic surfactants, such as sorbitan fatty acid ester, polyoxyethylene sorbitan fatty acid ester, sucrose fatty acid ester, polyoxyethylene fatty acid ester, polyoxyethylene resin acid ester, polyoxyethylene fatty acid diester, polyoxyethylene alkyl ether, polyoxyethylene alkyl aryl ether, polyoxyethylene alkyl phenyl ether, polyoxyethylene dialkyl phenyl ether, polyoxyethylene alkyl phenyl ether-formaldehyde condensates, polyoxyethylene-polyoxypropylene block copolymers, polystyrene-polyoxyethylene block polymers, alkyl polyoxyethylene-polypropylene block copolymer ether, polyoxyethylene alkylamine, polyoxyethylene fatty acid amide, polyoxyethylene fatty acid bis(phenyl ether), polyalkylene benzyl phenyl ether, polyoxyalkylene styryl phenyl ether, acetylene diol, polyoxyalkylene-added acetylene diol, polyoxyethylene ether-type silicone, ester-type silicone, fluorosurfactants, polyoxyethylene castor oil and polyoxyethylene hydrogenated castor oil; anionic surfactants, such as alkyl sulfates, polyoxyethylene alkyl ether sulfates, polyoxyethylene alkyl phenyl ether sulfates, polyoxyethylene styryl phenyl ether sulfates, alkylbenzene sulfonates, alkylaryl sulfonates, lignosulfonates, alkyl sulfosuccinates, naphthalene sulfonates, alkylnaphthalene sulfonates, salts of naphthalenesulfonic acid-formaldehyde condensates, salts of alkylnaphthalenesulfonic acid-formaldehyde condensates, fatty acid salts, polycarboxylic acid salts, polyacrylates, N-methyl-fatty acid sarcosinates, resinates, polyoxyethylene alkyl ether phosphates and polyoxyethylene alkyl phenyl ether phosphates; cationic surfactants including alkyl amine salts, such as lauryl amine hydrochloride, stearyl amine hydrochloride, oleyl amine hydrochloride, stearyl amine acetate, stearyl aminopropyl amine acetate, alkyl trimethyl ammonium chloride and alkyl dimethyl benzalkonium chloride; and amphoteric surfactants, such as amino acid-type or betaine-type amphoteric surfactants. One of these surfactants may be used alone, and also two or more of them may be used in combination.

Examples of the binders or the tackifiers include carboxymethyl cellulose or salts thereof, dextrin, soluble starch, xanthan gum, guar gum, sucrose, polyvinyl pyrrolidone, gum arabic, polyvinyl alcohol, polyvinyl acetate, sodium polyacrylate, polyethylene glycols with an average molecular weight of 6,000 to 20,000, polyethylene oxides with an average molecular weight of 100,000 to 5,000,000, phospholipids (for example, cephalin, lecithin, etc.), cellulose powder, dextrin, modified starch, polyaminocarboxylic acid chelating compounds, cross-linked polyvinyl pyrrolidone, maleic acid-styrene copolymers, (meth)acrylic acid copolymers, half esters of polyhydric alcohol polymer and dicarboxylic anhydride, water soluble polystyrene sulfonates, paraffin, terpene, polyamide resins, polyacrylates, polyoxyethylene, waxes, polyvinyl alkyl ether, alkylphenol-formaldehyde condensates and synthetic resin emulsions.

Examples of the thickeners include water soluble polymers, such as xanthan gum, guar gum, diutan gum, carboxymethyl cellulose, polyvinyl pyrrolidone, carboxyvinyl polymers, acrylic polymers, starch compounds and polysaccharides; and inorganic fine powders, such as high grade bentonite and fumed silica (white carbon).

Examples of the colorants include inorganic pigments, such as iron oxide, titanium oxide and Prussian blue; and organic dyes, such as alizarin dyes, azo dyes and metal phthalocyanine dyes.

Examples of the antifreezing agents include polyhydric alcohols, such as ethylene glycol, diethylene glycol, propylene glycol and glycerin.

Examples of the adjuvants serving to prevent caking or facilitate disintegration include polysaccharides (starch, alginic acid, mannose, galactose, etc.), polyvinyl pyrrolidone, fumed silica (white carbon), ester gum, petroleum resin, sodium tripolyphosphate, sodium hexametaphosphate, metal stearates, cellulose powder, dextrin, methacrylate copolymers, polyvinyl pyrrolidone, polyaminocarboxylic acid chelating compounds, sulfonated styrene-isobutylene-maleic anhydride copolymers and starch-polyacrylonitrile graft copolymers.

Examples of the stabilizing agents include desiccants, such as zeolite, quicklime and magnesium oxide; antioxidants, such as phenolic compounds, amine compounds, sulfur compounds and phosphoric acid compounds; and ultraviolet absorbers, such as salicylic acid compounds and benzophenone compounds.

Examples of the preservatives include potassium sorbate and 1,2-benzothiazolin-3-one.

Further, other adjuvants including functional spreading agents, activity enhancers such as metabolic inhibitors (piperonyl butoxide etc.), antifreezing agents (propylene glycol etc.), antioxidants (BHT etc.) and ultraviolet absorbers can also be used if needed.

The amount of the active ingredient compound in the agricultural and horticultural insecticide of the present invention can be adjusted as needed, and basically, the amount of the active ingredient compound is appropriately selected from the range of 0.01 to 90 parts by weight in 100 parts by weight of the agricultural and horticultural insecticide. For example, in the case where the agricultural and horticultural insecticide is a dust, a granule, an emulsifiable concentrate or a wettable powder, it is suitable that the amount of the active ingredient compound is 0.01 to 50 parts by weight (0.01 to 50% by weight relative to the total weight of the agricultural and horticultural insecticide).

The application rate of the agricultural and horticultural insecticide of the present invention may vary with various factors, for example, the purpose, the target pest, the growing conditions of crops, the tendency of pest infestation, the weather, the environmental conditions, the dosage form, the application method, the application site, the application timing, etc., but basically, the application rate of the active ingredient compound is appropriately selected from the range of 0.001 g to 10 kg, and preferably 0.01 g to 1 kg per 10 ares depending on the purpose.

Furthermore, for the expansion of the range of target pests and the appropriate time for pest control, or for dose reduction, the agricultural and horticultural insecticide of the present invention can be used after mixed with other agricultural and horticultural insecticides, acaricides, nematicides, microbicides, biopesticides and/or the like. Further, the agricultural and horticultural insecticide can be used after mixed with herbicides, plant growth regulators, fertilizers and/or the like depending on the situation.

Examples of such additional agricultural and horticultural insecticides, acaricides and nematicides used for the above-mentioned purposes include 3,5-xylyl methylcarbamate (XMC), crystalline protein toxins produced by Bacillus thuringiensis such as Bacillus thuringiensis aizawai, Bacillus thuringiensis israelensis, Bacillus thuringiensis japonensis, Bacillus thuringiensis kurstaki and Bacillus thuringiensis tenebrionis, BPMC, Bt toxin-derived insecticidal compounds, CPCBS (chlorfenson), DCIP (dichlorodiisopropyl ether), D-D (1,3-dichloropropene), DDT, NAC, O-4-dimethylsulfamoylphenyl O,O-diethyl phosphorothioate (DSP), O-ethyl O-4-nitrophenyl phenylphosphonothioate (EPN), tripropylisocyanurate (TPIC), acrinathrin, azadirachtin, azinphos-methyl, acequinocyl, acetamiprid, acetoprole, acephate, abamectin, avermectin-B, amidoflumet, amitraz, alanycarb, aldicarb, aldoxycarb, aldrin, alpha-endosulfan, alpha-cypermethrin, albendazole, allethrin, isazofos, isamidofos, isoamidofos isoxathion, isofenphos, isoprocarb (MIPC), ivermectin, imicyafos, imidacloprid, imiprothrin, indoxacarb, esfenvalerate, ethiofencarb, ethion, ethiprole, etoxazole, ethofenprox, ethoprophos, etrimfos, emamectin, emamectin-benzoate, endosulfan, empenthrin, oxamyl, oxydemeton-methyl, oxydeprofos (ESP), oxibendazole, oxfendazole, potassium oleate, sodium oleate, cadusafos, cartap, carbaryl, carbosulfan, carbofuran, gamma-cyhalothrin, xylylcarb, quinalphos, kinoprene, chinomethionat, cloethocarb, clothianidin, clofentezine, chromafenozide, chlorantraniliprole, chlorethoxyfos, chlordimeform, chlordane, chlorpyrifos, chlorpyrifos-methyl, chlorphenapyr, chlorfenson, chlorfenvinphos, chlorfluazuron, chlorobenzilate, chlorobenzoate, kelthane (dicofol), salithion, cyanophos (CYAP), diafenthiuron, diamidafos, cyantraniliprole, theta-cypermethrin, dienochlor, cyenopyrafen, dioxabenzofos, diofenolan, sigma-cypermethrin, dichlofenthion (ECP), cycloprothrin, dichlorvos (DDVP), disulfoton, dinotefuran, cyhalothrin, cyphenothrin, cyfluthrin, diflubenzuron, cyflumetofen, diflovidazin, cyhexatin, cypermethrin, dimethylvinphos, dimethoate, dimefluthrin, silafluofen, cyromazine, spinetoram, spinosad, spirodiclofen, spirotetramat, spiromesifen, sulfluramid, sulprofos, sulfoxaflor, zeta-cypermethrin, diazinon, tau-fluvalinate, dazomet, thiacloprid, thiamethoxam, thiodicarb, thiocyclam, thiosultap, thiosultap-sodium, thionazin, thiometon, deet, dieldrin, tetrachlorvinphos, tetradifon, tetramethylfluthrin, tetramethrin, tebupirimfos, tebufenozide, tebufenpyrad, tefluthrin, teflubenzuron, demeton-S-methyl, temephos, deltamethrin, terbufos, tralopyril, tralomethrin, transfluthrin, triazamate, triazuron, trichlamide, trichlorphon (DEP), triflumuron, tolfenpyrad, naled (BRP), nithiazine, nitenpyram, novaluron, noviflumuron, hydroprene, vaniliprole, vamidothion, parathion, parathion-methyl, halfenprox, halofenozide, bistrifluron, bisultap, hydramethylnon, hydroxy propyl starch, binapacryl, bifenazate, bifenthrin, pymetrozine, pyraclofos, pyrafluprole, pyridafenthion, pyridaben, pyridalyl, pyrifluquinazon, pyriprole, pyriproxyfen, pirimicarb, pyrimidifen, pirimiphos-methyl, pyrethrins, fipronil, fenazaquin, fenamiphos, bromopropylate, fenitrothion (MEP), fenoxycarb, fenothiocarb, phenothrin, fenobucarb, fensulfothion, fenthion (MPP), phenthoate (PAP), fenvalerate, fenpyroximate, fenpropathrin, fenbendazole, fosthiazate, formetanate, butathiofos, buprofezin, furathiocarb, prallethrin, fluacrypyrim, fluazinam, fluazuron, fluensulfone, flucycloxuron, flucythrinate, fluvalinate, flupyrazofos, flufenerim, flufenoxuron, flufenzine, flufenprox, fluproxyfen, flubrocythrinate, flubendiamide, flumethrin, flurimfen, prothiofos, protrifenbute, flonicamid, propaphos, propargite (BPPS), profenofos, profluthrin, propoxur (PHC), bromopropylate, beta-cyfluthrin, hexaflumuron, hexythiazox, heptenophos, permethrin, benclothiaz, bendiocarb, bensultap, benzoximate, benfuracarb, phoxim, phosalone, fosthiazate, fosthietan, phosphamidon, phosphocarb, phosmet (PMP), polynactins, formetanate, formothion, phorate, machine oil, malathion, milbemycin, milbemycin-A, milbemectin, mecarbam, mesulfenfos, methomyl, metaldehyde, metaflumizone, methamidophos, metam-ammonium, metam-sodium, methiocarb, methidathion (DMTP), methylisothiocyanate, methylneodecanamide, methylparathion, metoxadiazone, methoxychlor, methoxyfenozide, metofluthrin, methoprene, metolcarb, meperfluthrin, mevinphos, monocrotophos, monosultap, lambda-cyhalothrin, ryanodine, lufenuron, resmethrin, lepimectin, rotenone, levamisole hydrochloride, fenbutatin oxide, morantel tartarate, methyl bromide, tricyclohexyltin hydroxide (cyhexatin), calcium cyanamide, calcium polysulfide, sulfur and nicotine-sulfate.

Exemplary agricultural and horticultural microbicides used for the same purposes as above include aureofungin, azaconazole, azithiram, acypetacs, acibenzolar, acibenzolar-S-methyl, azoxystrobin, anilazine, amisulbrom, ampropylfos, ametoctradin, allyl alcohol, aldimorph, amobam, isotianil, isovaledione, isopyrazam, isoprothiolane, ipconazole, iprodione, iprovalicarb, iprobenfos, imazalil, iminoctadine, iminoctadine-albesilate, iminoctadine-triacetate, imibenconazole, uniconazole, uniconazole-P, echlomezole, edifenphos, etaconazole, ethaboxam, ethirimol, etem, ethoxyquin, etridiazole, enestroburin, epoxiconazole, oxadixyl, oxycarboxin, copper-8-quinolinolate, oxytetracycline, copper-oxinate, oxpoconazole, oxpoconazole-fumarate, oxolinic acid, octhilinone, ofurace, orysastrobin, soil fungicides such as metam-sodium, kasugamycin, carbamorph, carpropamid, carbendazim, carboxin, carvone, quinazamid, quinacetol, quinoxyfen, quinomethionate, captafol, captan, kiralaxyl, quinconazole, quintozene, guazatine, cufraneb, cuprobam, glyodin, griseofulvin, climbazole, cresol, kresoxim-methyl, chlozolinate, clotrimazole, chlobenthiazone, chloraniformethan, chloranil, chlorquinox, chloropicrin, chlorfenazole, chlorodinitronaphthalene, chlorothalonil, chloroneb, zarilamid, salicylanilide, cyazofamid, diethyl pyrocarbonate, diethofencarb, cyclafuramid, diclocymet, dichlozoline, diclobutrazol, dichlofluanid, cycloheximide, diclomezine, dicloran, dichlorophen, dichlone, disulfiram, ditalimfos, dithianon, diniconazole, diniconazole-M, zineb, dinocap, dinocton, dinosulfon, dinoterbon, dinobuton, dinopenton, dipyrithione, diphenylamine, difenoconazole, cyflufenamid, diflumetorim, cyproconazole, cyprodinil, cyprofuram, cypendazole, simeconazole, dimethirimol, dimethomorph, cymoxanil, dimoxystrobin, methyl bromide, ziram, silthiofam, streptomycin, spiroxamine, sultropen, sedaxane, zoxamide, dazomet, thiadiazin, tiadinil, thiadifluor, thiabendazole, tioxymid, thiochlorfenphim, thiophanate, thiophanate-methyl, thicyofen, thioquinox, chinomethionat, thifluzamide, thiram, decafentin, tecnazene, tecloftalam, tecoram, tetraconazole, debacarb, dehydroacetic acid, tebuconazole, tebufloquin, dodicin, dodine, dodecyl benzensulfonate bis-ethylene diamine copper(II) (DBEDC), dodemorph, drazoxolon, triadimenol, triadimefon, triazbutil, triazoxide, triamiphos, triarimol, trichlamide, tricyclazole, triticonazole, tridemorph, tributyltin oxide, triflumizole, trifloxystrobin, triforine, tolylfluanid, tolclofos-methyl, natamycin, nabam, nitrothal-isopropyl, nitrostyrene, nuarimol, copper nonylphenol sulfonate, halacrinate, validamycin, valifenalate, harpin protein, bixafen, picoxystrobin, picobenzamide, bithionol, bitertanol, hydroxyisoxazole, hydroxyisoxazole-potassium, binapacryl, biphenyl, piperalin, hymexazol, pyraoxystrobin, pyracarbolid, pyraclostrobin, pyrazophos, pyrametostrobin, pyriofenone, pyridinitril, pyrifenox, pyribencarb, pyrimethanil, pyroxychlor, pyroxyfur, pyroquilon, vinclozolin, famoxadone, fenapanil, fenamidone, fenaminosulf, fenarimol, fenitropan, fenoxanil, ferimzone, ferbam, fentin, fenpiclonil, fenpyrazamine, fenbuconazole, fenfuram, fenpropidin, fenpropimorph, fenhexamid, phthalide, buthiobate, butylamine, bupirimate, fuberidazole, blasticidin-S, furametpyr, furalaxyl, fluacrypyrim, fluazinam, fluoxastrobin, fluotrimazole, fluopicolide, fluopyram, fluoroimide, furcarbanil, fluxapyroxad, fluquinconazole, furconazole, furconazole-cis, fludioxonil, flusilazole, flusulfamide, flutianil, flutolanil, flutriafol, furfural, furmecyclox, flumetover, flumorph, proquinazid, prochloraz, procymidone, prothiocarb, prothioconazole, propamocarb, propiconazole, propineb, furophanate, probenazole, bromuconazole, hexachlorobutadiene, hexaconazole, hexylthiofos, bethoxazin, benalaxyl, benalaxyl-M, benodanil, benomyl, pefurazoate, benquinox, penconazole, benzamorf, pencycuron, benzohydroxamic acid, bentaluron, benthiazole, benthiavalicarb-isopropyl, penthiopyrad, penflufen, boscalid, phosdiphen, fosetyl, fosetyl-Al, polyoxins, polyoxorim, polycarbamate, folpet, formaldehyde, machine oil, maneb, mancozeb, mandipropamid, myclozolin, myclobutanil, mildiomycin, milneb, mecarbinzid, methasulfocarb, metazoxolon, metam, metam-sodium, metalaxyl, metalaxyl-M, metiram, methyl isothiocyanate, meptyldinocap, metconazole, metsulfovax, methfuroxam, metominostrobin, metrafenone, mepanipyrim, mefenoxam, meptyldinocap, mepronil, mebenil, iodomethane, rabenzazole, benzalkonium chloride, basic copper chloride, basic copper sulfate, inorganic microbicides such as silver, sodium hypochlorite, cupric hydroxide, wettable sulfur, calcium polysulfide, potassium hydrogen carbonate, sodium hydrogen carbonate, sulfur, copper sulfate anhydride, nickel dimethyldithiocarbamate, copper compounds such as copper-8-quinolinolate (oxine copper), zinc sulfate and copper sulfate pentahydrate.

Exemplary herbicides used for the same purposes as above include 1-naphthylacetamide, 2,4-PA, 2,3,6-TBA, 2,4,5-T, 2,4,5-TB, 2,4-D, 2,4-DB, 2,4-DEB, 2,4-DEP, 3,4-DA, 3,4-DB, 3,4-DP, 4-CPA, 4-CPB, 4-CPP, MCP, MCPA, MCPA-thioethyl, MCPB, ioxynil, aclonifen, azafenidin, acifluorfen, aziprotryne, azimsulfuron, asulam, acetochlor, atrazine, atraton, anisuron, anilofos, aviglycine, abscisic acid, amicarbazone, amidosulfuron, amitrole, aminocyclopyrachlor, aminopyralid, amibuzin, amiprophos-methyl, ametridione, ametryn, alachlor, allidochlor, alloxydim, alorac, isouron, isocarbamid, isoxachlortole, isoxapyrifop, isoxaflutole, isoxaben, isocil, isonoruron, isoproturon, isopropalin, isopolinate, isomethiozin, inabenfide, ipazine, ipfencarbazone, iprymidam, imazaquin, imazapic, imazapyr, imazamethapyr, imazamethabenz, imazamethabenz-methyl, imazamox, imazethapyr, imazosulfuron, indaziflam, indanofan, indolebutyric acid, uniconazole-P, eglinazine, esprocarb, ethametsulfuron, ethametsulfuron-methyl, ethalfluralin, ethiolate, ethychlozate-ethyl, ethidimuron, etinofen, ethephon, ethoxysulfuron, ethoxyfen, etnipromid, ethofumesate, etobenzanid, epronaz, erbon, endothal, oxadiazon, oxadiargyl, oxaziclomefone, oxasulfuron, oxapyrazon, oxyfluorfen, oryzalin, orthosulfamuron, orbencarb, cafenstrole, cambendichlor, carbasulam, carfentrazone, carfentrazone-ethyl, karbutilate, carbetamide, carboxazole, quizalofop, quizalofop-P, quizalofop-ethyl, xylachlor, quinoclamine, quinonamid, quinclorac, quinmerac, cumyluron, cliodinate, glyphosate, glufosinate, glufosinate-P, credazine, clethodim, cloxyfonac, clodinafop, clodinafop-propargyl, chlorotoluron, clopyralid, cloproxydim, cloprop, chlorbromuron, clofop, clomazone, chlomethoxynil, chlomethoxyfen, clomeprop, chlorazifop, chlorazine, cloransulam, chloranocryl, chloramben, cloransulam-methyl, chloridazon, chlorimuron, chlorimuron-ethyl, chlorsulfuron, chlorthal, chlorthiamid, chlortoluron, chlornitrofen, chlorfenac, chlorfenprop, chlorbufam, chlorflurazole, chlorflurenol, chlorprocarb, chlorpropham, chlormequat, chloreturon, chloroxynil, chloroxuron, chloropon, saflufenacil, cyanazine, cyanatryn, di-allate, diuron, diethamquat, dicamba, cycluron, cycloate, cycloxydim, diclosulam, cyclosulfamuron, dichlorprop, dichlorprop-P, dichlobenil, diclofop, diclofop-methyl, dichlormate, dichloralurea, diquat, cisanilide, disul, siduron, dithiopyr, dinitramine, cinidon-ethyl, dinosam, cinosulfuron, dinoseb, dinoterb, dinofenate, dinoprop, cyhalofop-butyl, diphenamid, difenoxuron, difenopenten, difenzoquat, cybutryne, cyprazine, cyprazole, diflufenican, diflufenzopyr, dipropetryn, cypromid, cyperquat, gibberellin, simazine, dimexano, dimethachlor, dimidazon, dimethametryn, dimethenamid, simetryn, simeton, dimepiperate, dimefuron, cinmethylin, swep, sulglycapin, sulcotrione, sulfallate, sulfentrazone, sulfosulfuron, sulfometuron, sulfometuron-methyl, secbumeton, sethoxydim, sebuthylazine, terbacil, daimuron, dazomet, dalapon, thiazafluron, thiazopyr, thiencarbazone, thiencarbazone-methyl, tiocarbazil, tioclorim, thiobencarb, thidiazimin, thidiazuron, thifensulfuron, thifensulfuron-methyl, desmedipham, desmetryn, tetrafluron, thenylchlor, tebutam, tebuthiuron, terbumeton, tepraloxydim, tefuryltrione, tembotrione, delachlor, terbacil, terbucarb, terbuchlor, terbuthylazine, terbutryn, topramezone, tralkoxydim, triaziflam, triasulfuron, tri-allate, trietazine, tricamba, triclopyr, tridiphane, tritac, tritosulfuron, triflusulfuron, triflusulfuron-methyl, trifluralin, trifloxysulfuron, tripropindan, tribenuron-methyl, tribenuron, trifop, trifopsime, trimeturon, naptalam, naproanilide, napropamide, nicosulfuron, nitralin, nitrofen, nitrofluorfen, nipyraclofen, neburon, norflurazon, noruron, barban, paclobutrazol, paraquat, parafluron, haloxydine, haloxyfop, haloxyfop-P, haloxyfop-methyl, halosafen, halosulfuron, halosulfuron-methyl, picloram, picolinafen, bicyclopyrone, bispyribac, bispyribac-sodium, pydanon, pinoxaden, bifenox, piperophos, hymexazol, pyraclonil, pyrasulfotole, pyrazoxyfen, pyrazosulfuron, pyrazosulfuron-ethyl, pyrazolate, bilanafos, pyraflufen-ethyl, pyriclor, pyridafol, pyrithiobac, pyrithiobac-sodium, pyridate, pyriftalid, pyributicarb, pyribenzoxim, pyrimisulfan, primisulfuron, pyriminobac-methyl, pyroxasulfone, pyroxsulam, fenasulam, phenisopham, fenuron, fenoxasulfone, fenoxaprop, fenoxaprop-P, fenoxaprop-ethyl, phenothiol, fenoprop, phenobenzuron, fenthiaprop, fenteracol, fentrazamide, phenmedipham, phenmedipham-ethyl, butachlor, butafenacil, butamifos, buthiuron, buthidazole, butylate, buturon, butenachlor, butroxydim, butralin, flazasulfuron, flamprop, furyloxyfen, prynachlor, primisulfuron-methyl, fluazifop, fluazifop-P, fluazifop-butyl, fluazolate, fluroxypyr, fluothiuron, fluometuron, fluoroglycofen, flurochloridone, fluorodifen, fluoronitrofen, fluoromidine, flucarbazone, flucarbazone-sodium, fluchloralin, flucetosulfuron, fluthiacet, fluthiacet-methyl, flupyrsulfuron, flufenacet, flufenican, flufenpyr, flupropacil, flupropanate, flupoxam, flumioxazin, flumiclorac, flumiclorac-pentyl, flumipropyn, flumezin, fluometuron, flumetsulam, fluridone, flurtamone, fluroxypyr, pretilachlor, proxan, proglinazine, procyazine, prodiamine, prosulfalin, prosulfuron, prosulfocarb, propaquizafop, propachlor, propazine, propanil, propyzamide, propisochlor, prohydrojasmon, propyrisulfuron, propham, profluazol, profluralin, prohexadione-calcium, propoxycarbazone, propoxycarbazone-sodium, profoxydim, bromacil, brompyrazon, prometryn, prometon, bromoxynil, bromofenoxim, bromobutide, bromobonil, florasulam, hexachloroacetone, hexazinone, pethoxamid, benazolin, penoxsulam, pebulate, beflubutamid, vernolate, perfluidone, bencarbazone, benzadox, benzipram, benzylaminopurine, benzthiazuron, benzfendizone, bensulide, bensulfuron-methyl, benzoylprop, benzobicyclon, benzofenap, benzofluor, bentazone, pentanochlor, benthiocarb, pendimethalin, pentoxazone, benfluralin, benfuresate, fosamine, fomesafen, foramsulfuron, forchlorfenuron, maleic hydrazide, mecoprop, mecoprop-P, medinoterb, mesosulfuron, mesosulfuron-methyl, mesotrione, mesoprazine, methoprotryne, metazachlor, methazole, metazosulfuron, methabenzthiazuron, metamitron, metamifop, metam, methalpropalin, methiuron, methiozolin, methiobencarb, methyldymron, metoxuron, metosulam, metsulfuron, metsulfuron-methyl, metflurazon, metobromuron, metobenzuron, methometon, metolachlor, metribuzin, mepiquat-chloride, mefenacet, mefluidide, monalide, monisouron, monuron, monochloroacetic acid, monolinuron, molinate, morfamquat, iodosulfuron, iodosulfuron-methyl-sodium, iodobonil, iodomethane, lactofen, linuron, rimsulfuron, lenacil, rhodethanil, calcium peroxide and methyl bromide.

Exemplary biopesticides used for the same purposes as above include viral formulations such as nuclear polyhedrosis viruses (NPV), granulosis viruses (GV), cytoplasmic polyhedrosis viruses (CPV) and entomopox viruses (EPV); microbial pesticides used as an insecticide or a nematicide, such as Monacrosporium phymatophagum, Steinernema carpocapsae, Steinernema kushidai and Pasteuria penetrans; microbial pesticides used as a microbicide, such as Trichoderma lignorum, Agrobacterium radiobactor, avirulent Erwinia carotovora and Bacillus subtilis; and biopesticides used as a herbicide, such as Xanthomonas campestris. Such a combined use of the agricultural and horticultural insecticide of the present invention with the foregoing biopesticide as a mixture can be expected to provide the same effect as above.

Other examples of the biopesticides include natural predators such as Encarsia formosa, Aphidius colemani, Aphidoletes aphidimyza, Diglyphus isaea, Dacnusa sibirica, Phytoseiulus persimilis, Amblyseius cucumeris and Orius sauteri; microbial pesticides such as Beauveria brongniartii; and pheromones such as (Z)-10-tetradecenyl acetate, (E,Z)-4,10-tetradecadienyl acetate, (Z)-8-dodecenyl acetate, (Z)-11-tetradecenyl acetate, (Z)-13-icosen-10-one and 14-methyl-1-octadecene.

### EXAMPLES

Hereinafter, representative Examples in connection with the present invention are shown, but the present invention is not limited thereto.

### Production Example 1

### Production of 2-(1-ethylsulfonylindol-2-yl)-3-methyl-6-pentafluoroethyl-3H-imidazo[4,5-c]pyridazine (Compound Number 1-33)

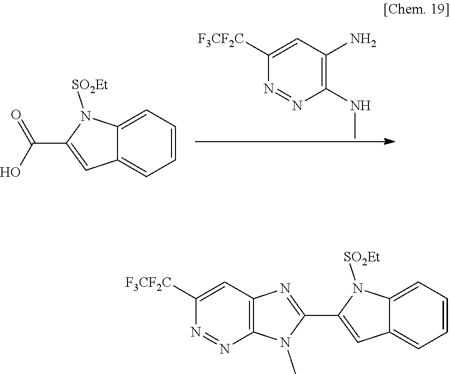

1-Ethylsulfonylindole-2-carboxylic acid (0.32 g, 1.3 mmol) was dissolved in 5 mL of toluene. To this, thionyl chloride (0.19 mL, 2.5 mmol) and three drops of dimethylformamide were added. The mixture was stirred at 100° C. for 1.5 hours, and the solvent was evaporated off in vacuo. The residue was dissolved in 3 mL of toluene. To this, 4-amino-3-methylamino-6-pentafluoroethyl pyridazine (0.31 g, 1.3 mmol) synthesized by the method described in the literature (WO 2016/039441) and pyridine (0.20 mL, 2.5 mmol) were added, and the mixture was stirred at 50° C. After the completion of the reaction, the reaction mixture was diluted with ethyl acetate and washed with water and a saturated aqueous sodium chloride solution. The organic layer was dried over anhydrous sodium sulfate, and the solvent was evaporated off. The residue was purified by silica gel column chromatography to give a crude product.

The crude product was dissolved in 5 mL of toluene and 5 mL of acetic acid, and the solution was heated under reflux for 30 minutes. After the completion of the reaction, the reaction mixture was diluted with ethyl acetate and washed with water and a saturated aqueous sodium chloride solution. The organic layer was dried over anhydrous sodium sulfate, and the solvent was evaporated off. The residue was purified by silica gel column chromatography to give the title compound (0.16 g, 0.34 mmol).

Yield: 27% (overall yield in 2 steps)
Physical property: Melting point 145 to 146° C.

### Production Example 2

### Production of 2-(1-ethylsulfonyl-6-fluoroindol-2-yl)-3-methyl-6-pentafluoroethyl-3H-imidazo[4,5-c]pyridazine (Compound Number 1-65)

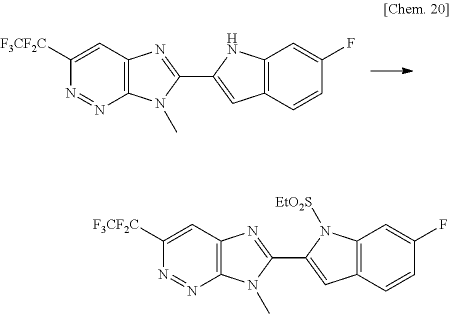

2-(6-Fluoro-1H-indol-2-yl)-3-methyl-6-pentafluoroethyl-3H-imidazo[4,5-c]pyridazine (45.7 mg, 0.119 mmol) was dissolved in 2.0 mL of N,N-dimethylformamide. To this, 60% sodium hydride (7.16 mg, 0.179 mmol) was added under ice-cooling, and the mixture was stirred for 30 minutes. A solution of ethylsulfonyl chloride (30.6 mg, 0.238 mmol) in N,N-dimethylformamide (1.0 mL) was further added dropwise at 0° C., and the mixture was stirred at room temperature for 20 hours. A saturated aqueous ammonium chloride solution was added to the reaction mixture, and extraction with ethyl acetate was performed, followed by washing with water and a saturated aqueous sodium chloride solution. The organic layer was dried over anhydrous sodium sulfate, and the solvent was evaporated off. The residue was purified by silica gel column chromatography to give the title compound (14.5 mg).

### Yield: 26%

Physical property: Melting point 82 to 86° C.

### Production Example 3

### Production of 2-(1-ethylsulfonyl-5-trifluoromethylindol-2-yl)-3-methyl-6-trifluoromethyl-3H-imidazo[4,5-b]pyridine (Compound Number 2-11)

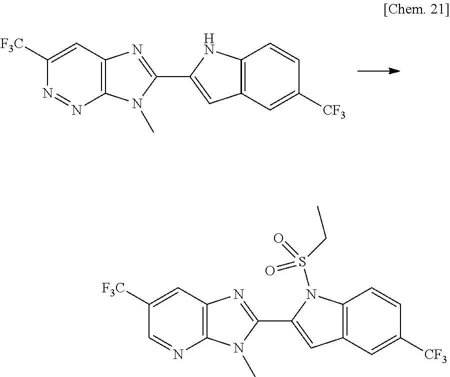 [SMILES: Cn1c(-c2cc3cc(C(F)(F)F)ccc3[nH]2)nc2cc(C(F)(F)F)nnc21 ; CCS(=O)(=O)n1c(-c2nc3cc(C(F)(F)F)cnc3n2C)cc2cc(C(F)(F)F)ccc21]

2-(5-Trifluoromethyl-1H-indol-2-yl)-3-methyl-6-trifluoromethyl-3H-imidazo[4,5-b]pyridine (100 mg, 0.260 mmol) was dissolved in 2.6 mL of N,N-dimethylformamide. To this, 60% sodium hydride (20.8 mg, 0.520 mmol) was added under ice-cooling, and the mixture was stirred for 15 minutes. Ethylsulfonyl chloride (73.5 mg, 0.572 mmol) was further added dropwise at 0° C., and the mixture was stirred at room temperature for 15 hours. A saturated aqueous ammonium chloride solution was added to the reaction mixture, and extraction with ethyl acetate was performed, followed by washing with water and a saturated aqueous sodium chloride solution. The organic layer was dried over anhydrous sodium sulfate, and the solvent was evaporated off. The residue was purified by silica gel column chromatography to give the title compound (69.1 mg).

### Yield: 50%

Physical property: Melting point 134 to 135° C.

### Production Example 4

### Production of 2-(1-ethylsulfonylindol-2-yl)-5-trifluoromethylthiobenzo[d]oxazole (Compound Number 3-1)

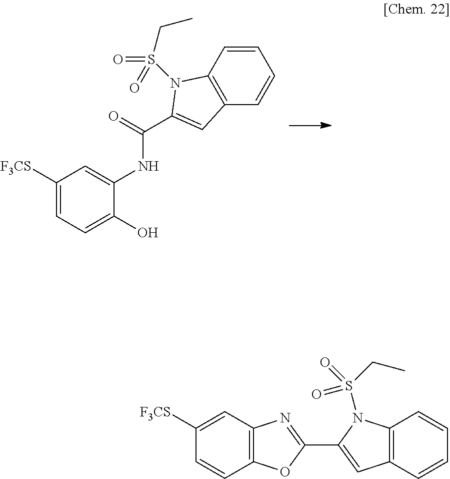

N-(2-Hydroxy-5-trifluoromethylthiophenyl)-1-ethylsulfonylindole-2-carboxylic amide (114 mg, 0.257 mmol) and triphenylphosphine (101 mg, 0.385 mmol) were dissolved in 2.5 mL of tetrahydrofuran. To this, bis(2-methoxyethyl) azodicarboxylate (0.270 mg, 4.50 mmol) was added at 60° C. The mixture was stirred at the same temperature for 1 hour and then allowed to cool. To the reaction mixture, an aqueous ammonium chloride solution was added. Extraction with ethyl acetate was performed, followed by washing with water and a saturated aqueous sodium chloride solution. The organic layer was dried over anhydrous sodium sulfate, and the solvent was evaporated off. The residue was purified by silica gel column chromatography to give the title compound (22 mg).

### Yield: 26%

Physical property: Melting point 118 to 119° C.

### Reference Example 1

### Production of methyl 1-ethylsulfonylindole-2-carboxylate

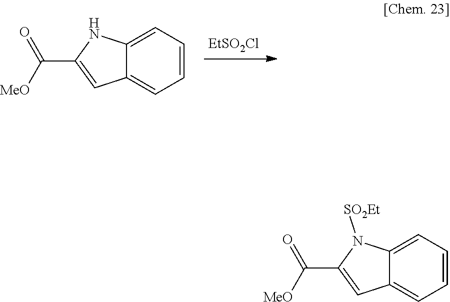

Methyl 1H-indole-2-carboxylate (3.5 g, 20 mmol) synthesized by the method described in the literature (WO 1999/007351) was dissolved in 30 mL of THF. To this, 60% sodium hydride (0.88 g, 22 mmol) was added under ice-cooling, and the mixture was stirred at ordinary temperature for 10 minutes. After ice-cooling again, ethanesulfonyl chloride (3.1 g, 24 mmol) was added dropwise, and the mixture was stirred at ordinary temperature for 15 minutes. Water was added under ice-cooling to stop the reaction. The reaction mixture was diluted with ethyl acetate and washed with water and a saturated aqueous sodium chloride solution. The organic layer was dried over anhydrous sodium sulfate, and the solvent was evaporated off. The residue was purified by silica gel column chromatography to give the title compound (2.4 g, 9.0 mmol).

### Yield: 45%

Physical property: Melting point 57 to 58° C.

### Reference Example 2

### Production of 1-ethylsulfonylindole-2-carboxylic Acid

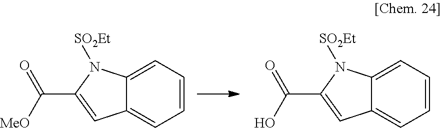

Methyl 1-ethylsulfonylindole-2-carboxylate (1.7 g, 6.2 mmol) was dissolved in 15 mL of THF. To this, a saturated aqueous sodium hydroxide solution containing sodium hydroxide (1.2 g, 30 mmol) was slowly added under ice-cooling, and the mixture was stirred at ordinary temperature. After the completion of the reaction, 2 M hydrochloric acid (15 mL) was added under ice-cooling. Extraction with ethyl acetate was performed, followed by washing with a saturated aqueous sodium chloride solution. The organic layer was dried over anhydrous sodium sulfate, and the solvent was evaporated off to give the title compound (1.5 g, 5.9 mmol) as a crude product.

Physical property: Melting point 136 to 140° C.

### Reference Example 3

### Production of methyl 1-methoxymethyl-6-fluoroindole-2-carboxylate

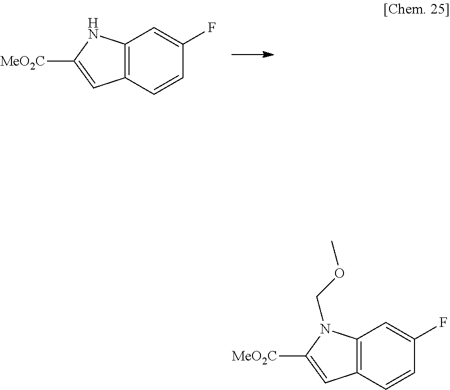 [SMILES: COC(=O)c1cc2ccc(F)cc2[nH]1 ; COCn1c(C(=O)OC)cc2ccc(F)cc21]

Methyl 6-fluoro-1H-indole-2-carboxylate (316 mg, 1.64 mmol) synthesized according to the method described in the literature (J. O. C., 2001, 66, 3474.) was dissolved in N,N-dimethylformamide (5.5 mL). To this, 60% sodium hydride (72.2 mg, 1.96 mmol) was added under ice-cooling, and the mixture was stirred for 10 minutes. Chloromethyl methyl ether (158 mg, 1.80 mmol) was added dropwise, and the mixture was stirred at room temperature for 1 hour. An aqueous ammonium chloride solution was added under ice-cooling, and extraction with ethyl acetate was performed, followed by washing with water and a saturated aqueous sodium chloride solution. The organic layer was dried over anhydrous sodium sulfate, and the solvent was evaporated off. The residue was purified by silica gel column chromatography to give the title compound (380 mg).

### Yield: 98%

Physical property: Melting point 85° C.

### Reference Example 4

### Production of N-(3-methylamino-6-pentafluoroethylpyridazin-4-yl)-1-methoxymethyl-6-fluoroindole-2-carboxylic amide

### [Chem. 26]

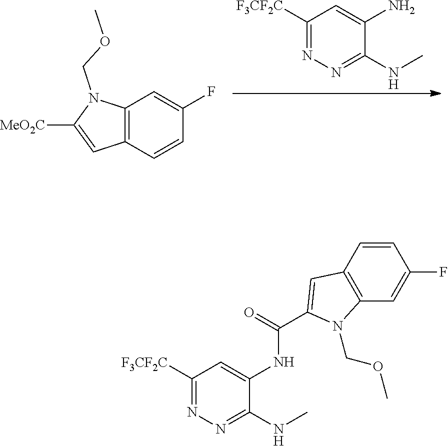

Methyl 1-methoxymethyl-6-fluoroindole-2-carboxylate (357 mg, 1.50 mmol) and 4-amino-3-methylamino-6-pentafluoroethyl pyridazine (413 mg, 1.43 mmol, purity 84%) synthesized by the method described in the literature (WO 2016/039441) were dissolved in 14.3 mL of tetrahydrofuran. To this, 60% sodium hydride (68.6 mg, 1.72 mmol) was added under ice-cooling. The mixture was stirred at the same temperature for 10 minutes, heated to 35° C., and further stirred for 3 hours. An aqueous ammonium chloride solution was added to the reaction mixture under ice-cooling, and extraction with ethyl acetate was performed, followed by washing with water and a saturated aqueous sodium chloride solution. The organic layer was dried over anhydrous sodium sulfate, and the solvent was evaporated off. The residue was purified by silica gel column chromatography to give the title compound (217 mg).

### Reference Example 5

### Production of 2-(1-methoxymethyl-6-fluoroindol-2-yl)-3-methyl-6-pentafluoroethyl-3H-imidazo[4,5-c]pyridazine

### [Chem. 27]

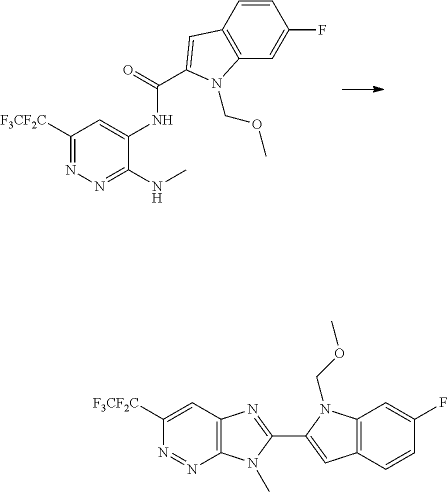

N-(3-Methylamino-6-pentafluoroethylpyridazin-4-yl)-1-methoxymethyl-6-fluoroindole-2-carboxylic amide (217 mg, 0.485 mmol) was dissolved in 8.0 mL of toluene and 1.5 mL of acetic acid. The solution was heated under reflux for 1.5 hours. The reaction mixture was allowed to cool and concentrated in vacuo. The resulting solid was washed to give the title compound (187 mg).

### Yield: 90%

Physical property: Melting point 165 to 166° C.

### Reference Example 6

### Production of 2-(6-fluoro-1H-indol-2-yl)-3-methyl-6-pentafluoroethyl-3H-imidazo[4,5-c]pyridazine

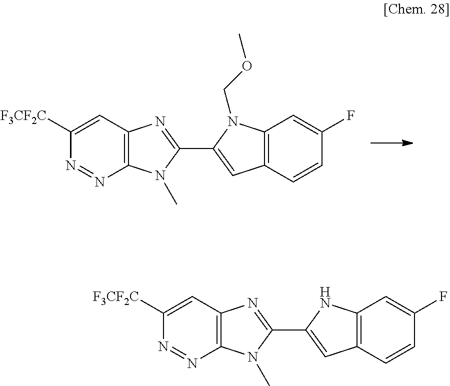

2-(1-Methoxymethyl-6-fluoroindol-2-yl)-3-methyl-6-heptafluoropropyl-3H-imidazo[4,5-c]pyridazine (165 mg, 0.384 mmol) was dissolved in 2.0 mL of trifluoroacetic acid. The solution was stirred at room temperature for 15 hours. The reaction mixture was concentrated in vacuo and diluted with ethyl acetate. To this, a saturated aqueous sodium bicarbonate solution was added. Extraction with ethyl acetate was performed, followed by washing with water and a saturated aqueous sodium chloride solution. The organic layer was dried over anhydrous sodium sulfate, and the solvent was evaporated off. The residue was purified by silica gel column chromatography to give the title compound (67.7 mg).

### Yield: 46%

Physical property: ¹H-NMR δ 9.62 (s, 1H), 8.06 (s, 1H), 7.71 (dd, 1H), 7.37 (dd, 1H), 7.19 (dd, 1H), 7.01 (ddd, 1H), 4.40 (s, 1H)

### Reference Example 7

### Production of methyl 1-benzyloxymethyl-5-trifluoromethylindole-2-carboxylate

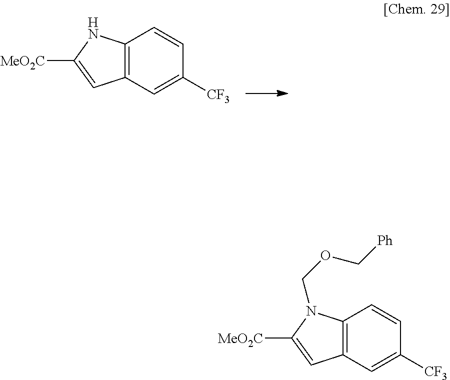 [SMILES: COC(=O)c1cc2cc(C(F)(F)F)ccc2[nH]1 ; COC(=O)c1cc2cc(C(F)(F)F)ccc2n1COCc1ccccc1]

Methyl 5-trifluoromethyl-1H-indole-2-carboxylate (1.30 g, 5.35 mmol) synthesized according to the method described in the literature (WO 2014/073627) was dissolved in 17.8 mL of N,N-dimethylformamide. To this, 60% sodium hydride (0.235 g, 5.89 mmol) was added under ice-cooling, and the mixture was stirred for 5 minutes. Benzyl chloromethyl ether (1.00 g, 6.42 mmol) was added dropwise, and the mixture was stirred at room temperature for 1 hour. An aqueous ammonium chloride solution was added under ice-cooling, and extraction with ethyl acetate was performed, followed by washing with water and a saturated aqueous sodium chloride solution. The organic layer was dried over anhydrous sodium sulfate, and the solvent was evaporated off. The residue was purified by silica gel column chromatography to give the title compound (1.99 g).

Yield: quantitative
Physical property: Refractive index 1.4900 (26.8° C.)

### Reference Example 8

### Production of N-(2-methylamino-5-trifluoromethylpyridin-3-yl)-1-benzyloxymethyl-5-trifluoromethylindole-2-carboxylic Amide

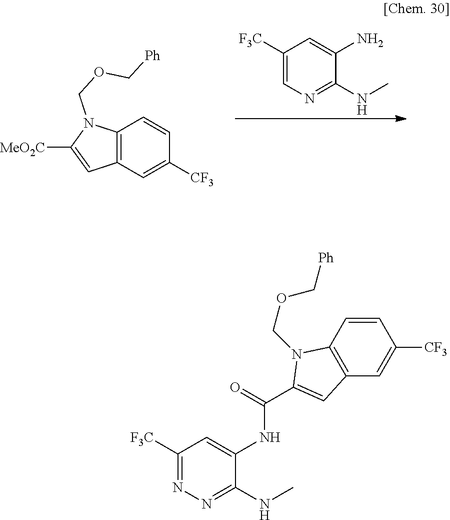 [SMILES: COC(=O)c1cc2cc(C(F)(F)F)ccc2n1COCc1ccccc1 ; CNc1ncc(C(F)(F)F)cc1N ; CNc1nnc(C(F)(F)F)cc1NC(=O)c1cc2cc(C(F)(F)F)ccc2n1COCc1ccccc1]

Methyl 1-benzyloxymethyl-5-trifluoromethylindole-2-carboxylate (1.27 g, 3.50 mmol) and 3-amino-2-methylamino-5-trifluoromethylpyridine (0.669 g, 3.50 mmol) were dissolved in 17.5 mL of tetrahydrofuran. To this, 60% sodium hydride (0.168 g, 4.20 mmol) was added under ice-cooling. The mixture was stirred at the same temperature for 15 minutes, heated to 35° C., and further stirred for 1 hour. An aqueous ammonium chloride solution was added to the reaction mixture under ice-cooling, and extraction with ethyl acetate was performed, followed by washing with water and a saturated aqueous sodium chloride solution. The organic layer was dried over anhydrous sodium sulfate, and the solvent was evaporated off. The residue was purified by silica gel column chromatography to give the title compound (0.95 g).

### Reference Example 9

### Production of 2-(5-trifluoromethyl-1H-indol-2-yl)-3-methyl-6-trifluoromethyl-3H-imidazo[4,5-b]pyridine

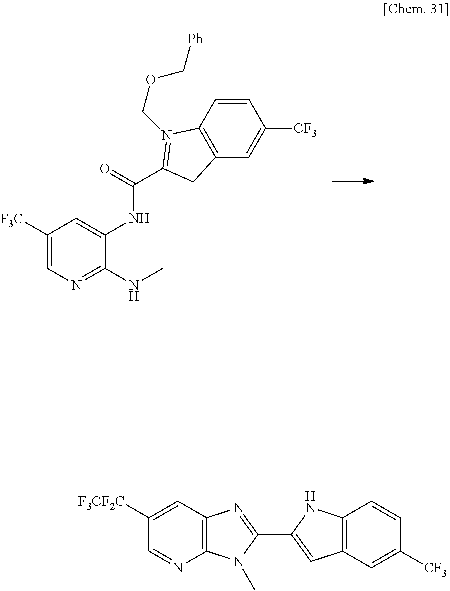

N-(2-Methylamino-5-trifluoromethylpyridin-3-yl)-1-benzyloxymethyl-5-trifluoromethylindole-2-carboxylic amide (0.95 g, 1.82 mmol) was dissolved in 10 mL of N-methyl-2-pyrrolidone. To this, p-toluenesulfonic acid monohydrate (1.04 g, 5.46 mmol) was added, and the mixture was stirred at 170° C. for 3 hours. The reaction mixture was allowed to cool. A saturated aqueous sodium bicarbonate solution was added to the reaction mixture, and extraction with ethyl acetate was performed, followed by washing with water and a saturated aqueous sodium chloride solution. The organic layer was dried over anhydrous sodium sulfate, and the solvent was evaporated off. The residue was purified by silica gel column chromatography. The resulting solid was washed with hexane to give the title compound (0.227 g).

### Yield: 33%

Physical property: ¹H-NMR δ 9.83 (s, 1H), 8.69 (d, 1H), 8.24 (d, 1H), 8.05 (s, 1H), 7.57 (s, 2H), 7.29 (d, 1H), 4.26 (s, 3H)

### Reference Example 10

### Production of N-(2-hydroxy-5-trifluoromethylthiophenyl)-1-ethylsulfonylindole-2-carboxylic Amide

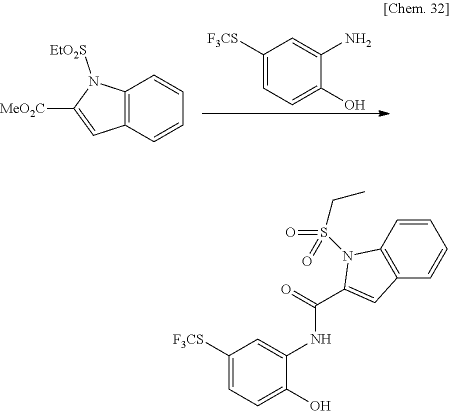

1-Ethylsulfonylindole-2-carboxylic acid (249 mg, 0.98 mmol), 2-hydroxy-5-trifluoromethyl thioaniline (205 mg, 0.98 mmol) and 1-(3-dimethylaminopropyl)-3-ethylcarbodiimide hydrochloride (226 mg, 1.18 mmol) were dissolved in 5.0 mL of pyridine. The solution was stirred at room temperature for 14 hours. Water was added to the reaction mixture, and extraction with ethyl acetate was performed, followed by washing with water and a saturated aqueous sodium chloride solution. The organic layer was dried over anhydrous sodium sulfate, and the solvent was evaporated off. The residue was purified by silica gel column chromatography to give the title compound (114 mg).

### Yield: 26%

Hereinafter, formulation examples are shown, but the present invention is not limited thereto. In the formulation examples, “part” means part by weight.

### Formulation Example 1

| | |
|---|---|
| Compound of the present invention | 10 parts |
| Xylene | 70 parts |
| N-methylpyrrolidone | 10 parts |
| Mixture of polyoxyethylene nonylphenyl | 10 parts |
| ether and calcium alkylbenzene sulfonate | |

The above ingredients are uniformly mixed for dissolution to give an emulsifiable concentrate formulation.

### Formulation Example 2

| | |
|---|---|
| Compound of the present invention | 3 parts |
| Clay powder | 82 parts |
| Diatomite powder | 15 parts |

The above ingredients are uniformly mixed and then pulverized to give a dust formulation.

### Formulation Example 3

| | |
|---|---|
| Compound of the present invention | 5 parts |
| Mixture of bentonite powder and clay powder | 90 parts |
| Calcium lignosulfonate | 5 parts |

The above ingredients are uniformly mixed. After addition of an appropriate volume of water, the mixture is kneaded, granulated and dried to give a granular formulation.

### Formulation Example 4

| | |
|---|---|
| Compound of the present invention | 20 parts |
| Kaolin and synthetic high-dispersion | 75 parts |
| silicic acid | |
| Mixture of polyoxyethylene nonylphenyl | 5 parts |
| ether and calcium alkylbenzene sulfonate | |

The above ingredients are uniformly mixed and then pulverized to give a wettable powder formulation.

Hereinafter, biological test examples are shown, but the present invention is not limited thereto.

### Test Example 1

Test for Control Efficacy on Myzus persicae

Chinese cabbage plants were planted in plastic pots (diameter: 8 cm, height: 8 cm), Green peach aphids (Myzus persicae) were propagated on the plants, and the number of surviving Green peach aphids in each pot was counted. The compound represented by the general formula (1) of the present invention or salts thereof were separately dispersed in water and diluted to 500 ppm. The agrochemical dispersions were applied to the foliage of the potted Chinese cabbage plants. After the plants were air-dried, the pots were kept in a greenhouse. At 6 days after the foliar application, the number of surviving Green peach aphids on the Chinese cabbage plant in each pot was counted, the control rate was calculated according to the formula shown below, and the control efficacy was evaluated according to the criteria shown below.

Control rate=100−{(T×Ca)/(Ta×C)}×100 [Math. 1]

Ta: the number of survivors before the foliar application in a treatment plot
T: the number of survivors after the foliar application in a treatment plot
Ca: the number of survivors before the foliar application in a non-treatment plot
C: the number of survivors after the foliar application in a non-treatment plot

### Criteria

A: the control rate is 100%.
B: the control rate is 90 to 99%.
C: the control rate is 80 to 89%.
D: the control rate is 50 to 79%.

As a result, among the compounds represented by the general formula (1) of the present invention, the compounds 1-1, 1-33, 1-35, 1-36, 1-43, 1-63, 1-65, 2-1, 2-3, 2-5, 2-10, 2-11, 2-31, 2-64, 2-65, 2-66, 2-67, 6-1, 6-2, 6-3, 7-1, 8-18 and 8-20 showed excellent insecticidal efficacy on Myzus persicae, which was evaluated as A.

### Test Example 2

Insecticidal Test on Laodelphax striatellus

The compounds represented by the general formula (1) of the present invention or salts thereof were separately dispersed in water and diluted to 500 ppm. Rice plant seedlings (variety: Nihonbare) were dipped in the agrochemical dispersions for 30 seconds. After air-dried, each seedling was put into a separate glass test tube and inoculated with ten 3rd-instar larvae of Laodelphax striatellus, and then the glass test tubes were capped with cotton plugs. At 8 days after the inoculation, the numbers of surviving larvae and dead larvae were counted, the corrected mortality rate was calculated according to the formula shown below, and the insecticidal efficacy was evaluated according to the criteria shown below.

Corrected mortality rate (%)=100×(Survival rate in a non-treatment plot−Survival rate in a treatment plot)/Survival rate in a non-treatment plot [Math. 2]

### Criteria

A: the corrected mortality rate is 100%.
B: the corrected mortality rate is 90 to 99%.
C: the corrected mortality rate is 80 to 89%.
D: the corrected mortality rate is 50 to 79%.

As a result, the compounds 1-33, 1-35, 1-36, 1-43, 1-63, 1-65, 1-66, 2-1, 2-3, 2-5, 2-10, 2-11, 2-31, 2-64, 2-65, 2-67, 6-1, 7-1 and 8-20 of the present invention showed the activity level evaluated as A.

### Test Example 3

Insecticidal Test on Plutella xylostella

Adults of Plutella xylostella were released onto Chinese cabbage seedlings and allowed to lay eggs thereon. At 2 days after the release of the adults, the Chinese cabbage seedlings with laid eggs were dipped for about 30 seconds in agrochemical formulations diluted to 500 ppm, each of which contained a different compound represented by the general formula (1) of the present invention as an active ingredient. After air-dried, the seedlings were kept in a thermostatic chamber at 25° C. At 6 days after the dip treatment, the number of hatched larvae per plot was counted, the mortality rate was calculated according to the formula shown below, and the insecticidal efficacy was evaluated according to the criteria of Test Example 2. This test was conducted in triplicate using 10 adults of Plutella xylostella per plot.

Corrected mortality rate (%)=100×(Number of hatched larvae in a non-treatment plot−Number of hatched larvae in a treatment plot)/Number of hatched larvae in a non-treatment plot [Math. 3]

As a result, the compounds 1-1, 1-33, 1-35, 1-36, 1-43, 1-63, 1-65, 1-66, 2-1, 2-3, 2-5, 2-10, 2-11, 2-15, 2-31, 2-64, 2-65, 2-66, 2-67, 3-1, 3-4, 3-11, 6-1, 6-2, 6-3, 7-1, 8-18, 8-19, 8-20, 8-21 and 8-22 of the present invention showed the activity level evaluated as A.

### INDUSTRIAL APPLICABILITY

The compound of the present invention or a salt thereof is highly effective as an agricultural and horticultural insecticide.

1. A compound represented by the general formula (1): 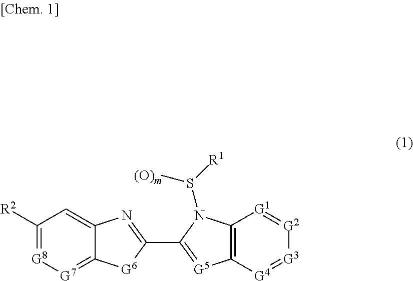
{wherein
R¹ represents:
(a1) a (C₁-C₆) alkyl group;
(a2) a (C₃-C₆) cycloalkyl group;
(a3) a (C₂-C₆) alkenyl group; or
(a4) a (C₂-C₆) alkynyl group,
R² represents:
(b1) a halogen atom;
(b2) a cyano group;
(b3) a nitro group;
(b4) a (C₁-C₆) alkyl group;
(b5) a (C₁-C₆) alkoxy group;
(b6) a (C₂-C₆) alkenyloxy group;
(b7) a (C₂-C₆) alkynyloxy group;
(b8) a halo (C₁-C₆) alkyl group;
(b9) a halo (C₁-C₆) alkoxy group;
(b10) a halo (C₂-C₆) alkenyloxy group;
(b11) a halo (C₂-C₆) alkynyloxy group;
(b12) a (C₁-C₆) alkylthio group;
(b13) a (C₁-C₆) alkylsulfinyl group;
(b14) a (C₁-C₆) alkylsulfonyl group;
(b15) a halo (C₁-C₆) alkylthio group;
(b16) a halo (C₁-C₆) alkylsulfinyl group; or
(b17) a halo (C₁-C₆) alkylsulfonyl group,
G¹, G², G³ and G⁴ independently represent C—R³ or N
(wherein
each R³ independently represents:
(c1) a hydrogen atom;
(c2) a halogen atom;
(c3) a hydroxyl group;
(c4) a formyl group;
(c5) a cyano group;
(c6) a (C₁-C₆) alkyl group;
(c7) a (C₃-C₆) cycloalkyl group;
(c8) a (C₁-C₆) alkoxy group;
(c9) a halo (C₁-C₆) alkyl group;
(c10) a halo (C₃-C₆) cycloalkyl group;
(c11) a halo (C₁-C₆) alkoxy group;
(c12) a (C₁-C₆) alkylthio group;
(c13) a (C₁-C₆) alkylsulfinyl group;
(c14) a (C₁-C₆) alkylsulfonyl group;
(c15) a halo (C₁-C₆) alkylthio group;
(c16) a halo (C₁-C₆) alkylsulfinyl group;
(c17) a halo (C₁-C₆) alkylsulfonyl group;
(c18) a (C₃-C₆) cycloalkyl (C₁-C₆) alkoxy group;
(c19) an R⁹(R¹⁰)N group (wherein R⁹ and R¹⁰ independently represent a hydrogen atom; a (C₁-C₆) alkyl group; a (C₃-C₆) cycloalkyl (C₁-C₆) alkyl group; a halo (C₁-C₆) alkyl group; a (C₁-C₆) alkylcarbonyl group; a (C₁-C₆) alkoxycarbonyl group; or a phenylcarbonyl group);
(c20) a mono (C₁-C₆) alkylaminocarbonyl NH group;
(c21) a mono (C₁-C₆) alkylaminothiocarbonyl NH group;
(c22) an R¹⁰ON═C(R⁹) group (wherein R⁹ and R¹⁰ are as defined above);
(c23) a carboxyl group;
(c24) a (C₁-C₆) alkoxycarbonyl group;
(c25) an R⁹(R¹⁰)N carbonyl group (wherein R⁹ and R¹⁰ are as defined above);
(c26) an aryl group;
(c27) an aryl group having, on the ring, 1 to 5 substituting groups independently selected from the group consisting of a halogen atom, a (C₁-C₆) alkyl group, a halo (C₁-C₆) alkyl group, a (C₁-C₆) alkoxy group, a halo (C₁-C₆) alkoxy group, a (C₁-C₆) alkylthio group, a halo (C₁-C₆) alkylthio group, a (C₁-C₆) alkylsulfinyl group, a halo (C₁-C₆) alkylsulfinyl group, a (C₁-C₆) alkylsulfonyl group and a halo (C₁-C₆) alkylsulfonyl group;
(c28) an aryl (C₁-C₆) alkyl group;
(c29) an aryl (C₁-C₆) alkyl group having, on the ring, 1 to 5 substituting groups independently selected from the group consisting of a halogen atom, a (C₁-C₆) alkyl group, a halo (C₁-C₆) alkyl group, a (C₁-C₆) alkoxy group, a halo (C₁-C₆) alkoxy group, a (C₁-C₆) alkylthio group, a halo (C₁-C₆) alkylthio group, a (C₁-C₆) alkylsulfinyl group, a halo (C₁-C₆) alkylsulfinyl group, a (C₁-C₆) alkylsulfonyl group and a halo (C₁-C₆) alkylsulfonyl group;
(c30) an aryl (C₁-C₆) alkoxy group;
(c31) an aryl (C₁-C₆) alkoxy group having, on the ring, 1 to 5 substituting groups independently selected from the group consisting of a halogen atom, a (C₁-C₆) alkyl group, a halo (C₁-C₆) alkyl group, a (C₁-C₆) alkoxy group, a halo (C₁-C₆) alkoxy group, a (C₁-C₆) alkylthio group, a halo (C₁-C₆) alkylthio group, a (C₁-C₆) alkylsulfinyl group, a halo (C₁-C₆) alkylsulfinyl group, a (C₁-C₆) alkylsulfonyl group and a halo (C₁-C₆) alkylsulfonyl group;
(c32) an aryl (C₁-C₆) alkyl NH group;
(c33) an aryl (C₁-C₆) alkyl NH group having, on the ring, 1 to 5 substituting groups independently selected from the group consisting of a halogen atom, a (C₁-C₆) alkyl group, a halo (C₁-C₆) alkyl group, a (C₁-C₆) alkoxy group, a halo (C₁-C₆) alkoxy group, a (C₁-C₆) alkylthio group, a halo (C₁-C₆) alkylthio group, a (C₁-C₆) alkylsulfinyl group, a halo (C₁-C₆) alkylsulfinyl group, a (C₁-C₆) alkylsulfonyl group and a halo (C₁-C₆) alkylsulfonyl group;
(c34) a heterocyclic group;
(c35) a heterocyclic group having, on the ring, 1 to 3 substituting groups independently selected from the group consisting of a halogen atom, a (C₁-C₆) alkyl group, a halo (C₁-C₆) alkyl group, a (C₁-C₆) alkoxy group, a halo (C₁-C₆) alkoxy group, a (C₁-C₆) alkylthio group, a halo (C₁-C₆) alkylthio group, a (C₁-C₆) alkylsulfinyl group, a halo (C₁-C₆) alkylsulfinyl group, a (C₁-C₆) alkylsulfonyl group and a halo (C₁-C₆) alkylsulfonyl group; or
(c36) a (C₁-C₆) alkoxycarbonyl NH group),
G⁵ represents C—R⁴ or N
(wherein
R⁴ represents:
(d1) a hydrogen atom;
(d2) a halogen atom;
(d3) a (C₁-C₆) alkyl group;
(d4) a (C₃-C₆) cycloalkyl group;
(d5) a (C₂-C₆) alkenyl group;
(d6) a (C₂-C₆) alkynyl group;
(d7) a (C₁-C₆) alkoxy group;
(d8) a halo (C₁-C₆) alkyl group;
(d9) a halo (C₃-C₆) cycloalkyl group;
(d10) a halo (C₂-C₆) alkenyl group;
(d11) a halo (C₂-C₆) alkynyl group;
(d12) a halo (C₁-C₆) alkoxy group;
(d13) a cyano group;
(d14) an R⁹(R¹⁰)N group (wherein R⁹ and R¹⁰ are as defined above);
(d15) a (C₁-C₆) alkylthio group;
(d16) a (C₁-C₆) alkylsulfinyl group;
(d17) a (C₁-C₆) alkylsulfonyl group;
(d18) a (C₁-C₆) alkylthio group;
(d19) a formyl group;
(d20) a (C₁-C₆) alkoxycarbonyl group;
(d21) an R⁹(R¹⁰)N carbonyl group (wherein R⁹ and R¹⁰ are as defined above); or
(d22) an R¹⁰ON═C(R⁹) group (wherein R⁹ and R¹⁰ are as defined above)),
G⁶ represents N—R⁵; O; or S
(wherein
R⁵ represents:
(e1) a hydrogen atom;
(e2) a (C₁-C₆) alkyl group;
(e3) a (C₃-C₆) cycloalkyl group;
(e4) a (C₂-C₆) alkenyl group; or
(e5) a (C₂-C₆) alkynyl group),
G⁷ and G⁸ independently represent C—H or N, and
m represents an integer of 0 to 2}, or
a salt thereof.

2-14. (canceled)

15. The compound or the salt according to claim 1, wherein
R¹ is (a1) a (C₁-C₆) alkyl group,
R² is:
(b8) a halo (C₁-C₆) alkyl group;
(b15) a halo (C₁-C₆) alkylthio group; or
(b17) a halo (C₁-C₆) alkylsulfonyl group,
each R³ is independently
(c1) a hydrogen atom;
(c2) a halogen atom;
(c4) a formyl group;
(c5) a cyano group;
(c6) a (C₁-C₆) alkyl group;
(c7) a (C₃-C₆) cycloalkyl group;
(c8) a (C₁-C₆) alkoxy group;
(c9) a halo (C₁-C₆) alkyl group;
(c11) a halo (C₁-C₆) alkoxy group;
(c12) a (C₁-C₆) alkylthio group;
(c13) a (C₁-C₆) alkylsulfinyl group;
(c14) a (C₁-C₆) alkylsulfonyl group;
(c15) a halo (C₁-C₆) alkylthio group;
(c19) an R⁹(R¹⁰)N group (wherein R⁹ and R¹⁰ are as defined above);
(c22) an R¹⁰ON═C(R⁹) group (wherein R⁹ and R¹⁰ are as defined above);
(c24) a (C₁-C₆) alkoxycarbonyl group;
(c25) an R⁹(R¹⁰)N carbonyl group (wherein R⁹ and R¹⁰ are as defined above);
(c27) an aryl group having, on the ring, 1 to 5 substituting groups independently selected from the group consisting of a halogen atom, a (C₁-C₆) alkyl group, a halo (C₁-C₆) alkyl group, a (C₁-C₆) alkoxy group, a halo (C₁-C₆) alkoxy group, a (C₁-C₆) alkylthio group, a halo (C₁-C₆) alkylthio group, a (C₁-C₆) alkylsulfinyl group, a halo (C₁-C₆) alkylsulfinyl group, a (C₁-C₆) alkylsulfonyl group and a halo (C₁-C₆) alkylsulfonyl group; or
(c36) a (C₁-C₆) alkoxycarbonyl NH group,
R⁴ is:
(d1) a hydrogen atom;
(d2) a halogen atom;
(d3) a (C₁-C₆) alkyl group;
(d4) a (C₃-C₆) cycloalkyl group;
(d7) a (C₁-C₆) alkoxy group;
(d12) a halo (C₁-C₆) alkoxy group;
(d13) a cyano group;
(d14) an R⁹(R¹⁰)N group (wherein R⁹ and R¹⁰ are as defined above);
(d15) a (C₁-C₆) alkylthio group;
(d16) a (C₁-C₆) alkylsulfinyl group;
(d17) a (C₁-C₆) alkylsulfonyl group;
(d18) a (C₁-C₆) alkylthio group;
(d19) a formyl group;
(d20) a (C₁-C₆) alkoxycarbonyl group;
(d21) an R⁹(R¹⁰)N carbonyl group (wherein R⁹ and R¹⁰ are as defined above); or
(d22) an R¹⁰ON═C(R⁹) group (wherein R⁹ and R¹⁰ are as defined above),
G⁶ is N—R⁵ or O, and
R⁵ is (e2) a (C₁-C₆) alkyl group.

16. The compound or the salt according to claim 1, wherein
R² is:
(b8) a halo (C₁-C₆) alkyl group or
(b15) a halo (C₁-C₆) alkylthio group,
each R³ is independently:
(c1) a hydrogen atom;
(c2) a halogen atom;
(c8) a (C₁-C₆) alkoxy group;
(c9) a halo (C₁-C₆) alkyl group; or
(c11) a halo (C₁-C₆) alkoxy group, and
R⁴ is:
(d1) a hydrogen atom or
(d2) a halogen atom.

17. An insecticide comprising the compound or the salt according to claim 1 as an active ingredient.

18. The insecticide of claim 17, wherein said insecticide is an agricultural or horticultural insecticide.

19. The insecticide of claim 17, wherein said insecticide is an animal ectoparasite control agent.

20. A method for using an insecticide, comprising contacting plants or soil with an effective amount of the insecticide according to claim 17.

21. A condensed heterocyclic compound represented by the general formula (1): 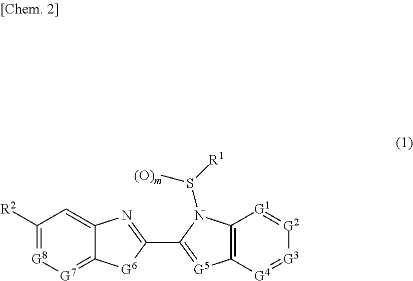
{wherein
R¹ represents:
(a1) a (C₁-C₆) alkyl group;
(a2) a (C₃-C₆) cycloalkyl group;
(a3) a (C₂-C₆) alkenyl group; or
(a4) a (C₂-C₆) alkynyl group,
R² represents:
(b1) a halogen atom;
(b2) a cyano group;
(b3) a nitro group;
(b4) a (C₁-C₆) alkyl group;
(b5) a (C₁-C₆) alkoxy group;
(b6) a (C₂-C₆) alkenyloxy group;
(b7) a (C₂-C₆) alkynyloxy group;
(b8) a halo (C₁-C₆) alkyl group;
(b9) a halo (C₁-C₆) alkoxy group;
(b10) a halo (C₂-C₆) alkenyloxy group;
(b11) a halo (C₂-C₆) alkynyloxy group;
(b12) a (C₁-C₆) alkylthio group;
(b13) a (C₁-C₆) alkylsulfinyl group;
(b14) a (C₁-C₆) alkylsulfonyl group;
(b15) a halo (C₁-C₆) alkylthio group;
(b16) a halo (C₁-C₆) alkylsulfinyl group; or
(b17) a halo (C₁-C₆) alkylsulfonyl group,
G¹, G², G³ and G⁴ may be the same or different and each represent C—R³ or N
(wherein
R³s may be the same or different and represents:
(c1) a hydrogen atom;
(c2) a halogen atom;
(c3) a hydroxyl group;
(c4) a formyl group;
(c5) a cyano group;
(c6) a (C₁-C₆) alkyl group;
(c7) a (C₃-C₆) cycloalkyl group;
(c8) a (C₁-C₆) alkoxy group;
(c9) a halo (C₁-C₆) alkyl group;
(c10) a halo (C₃-C₆) cycloalkyl group;
(c11) a halo (C₁-C₆) alkoxy group;
(c12) a (C₁-C₆) alkylthio group;
(c13) a (C₁-C₆) alkylsulfinyl group;
(c14) a (C₁-C₆) alkylsulfonyl group;
(c15) a halo (C₁-C₆) alkylthio group;
(c16) a halo (C₁-C₆) alkylsulfinyl group;
(c17) a halo (C₁-C₆) alkylsulfonyl group;
(c18) a (C₃-C₆) cycloalkyl (C₁-C₆) alkoxy group;
(c19) an R⁹(R¹⁰)N group (wherein R⁹ and R¹⁰ may be the same or different and each represent a hydrogen atom; a (C₁-C₆) alkyl group; a (C₃-C₆) cycloalkyl (C₁-C₆) alkyl group; a halo (C₁-C₆) alkyl group; a (C₁-C₆) alkylcarbonyl group; a (C₁-C₆) alkoxycarbonyl group; or a phenylcarbonyl group);
(c20) a mono (C₁-C₆) alkylaminocarbonyl NH group;
(c21) a mono (C₁-C₆) alkylaminothiocarbonyl NH group;
(c22) a C(R⁹)═NOR¹⁰ group (wherein R⁹ and R¹⁰ are as defined above);
(c23) a carboxyl group;
(c24) a (C₁-C₆) alkoxycarbonyl group;
(c25) an R⁹(R¹⁰)N carbonyl group (wherein R⁹ and R¹⁰ are as defined above);
(c26) an aryl group;
(c27) an aryl group having, on the ring, 1 to 5 substituting groups which may be the same or different and are selected from the group consisting of a halogen atom, a (C₁-C₆) alkyl group, a halo (C₁-C₆) alkyl group, a (C₁-C₆) alkoxy group, a halo (C₁-C₆) alkoxy group, a (C₁-C₆) alkylthio group, a halo (C₁-C₆) alkylthio group, a (C₁-C₆) alkylsulfinyl group, a halo (C₁-C₆) alkylsulfinyl group, a (C₁-C₆) alkylsulfonyl group and a halo (C₁-C₆) alkylsulfonyl group;
(c28) an aryl (C₁-C₆) alkyl group;
(c29) an aryl (C₁-C₆) alkyl group having, on the ring, 1 to 5 substituting groups which may be the same or different and are selected from the group consisting of a halogen atom, a (C₁-C₆) alkyl group, a halo (C₁-C₆) alkyl group, a (C₁-C₆) alkoxy group, a halo (C₁-C₆) alkoxy group, a (C₁-C₆) alkylthio group, a halo (C₁-C₆) alkylthio group, a (C₁-C₆) alkylsulfinyl group, a halo (C₁-C₆) alkylsulfinyl group, a (C₁-C₆) alkylsulfonyl group and a halo (C₁-C₆) alkylsulfonyl group;
(c30) an aryl (C₁-C₆) alkoxy group;
(c31) an aryl (C₁-C₆) alkoxy group having, on the ring, 1 to 5 substituting groups which may be the same or different and are selected from the group consisting of a halogen atom, a (C₁-C₆) alkyl group, a halo (C₁-C₆) alkyl group, a (C₁-C₆) alkoxy group, a halo (C₁-C₆) alkoxy group, a (C₁-C₆) alkylthio group, a halo (C₁-C₆) alkylthio group, a (C₁-C₆) alkylsulfinyl group, a halo (C₁-C₆) alkylsulfinyl group, a (C₁-C₆) alkylsulfonyl group and a halo (C₁-C₆) alkylsulfonyl group;
(c32) an aryl (C₁-C₆) alkyl NH group;
(c33) an aryl (C₁-C₆) alkyl NH group having, on the ring, 1 to 5 substituting groups which may be the same or different and are selected from the group consisting of a halogen atom, a (C₁-C₆) alkyl group, a halo (C₁-C₆) alkyl group, a (C₁-C₆) alkoxy group, a halo (C₁-C₆) alkoxy group, a (C₁-C₆) alkylthio group, a halo (C₁-C₆) alkylthio group, a (C₁-C₆) alkylsulfinyl group, a halo (C₁-C₆) alkylsulfinyl group, a (C₁-C₆) alkylsulfonyl group and a halo (C₁-C₆) alkylsulfonyl group;
(c34) a heterocyclic group; or
(c35) a heterocyclic group having, on the ring, 1 to 3 substituting groups which may be the same or different and are selected from the group consisting of a halogen atom, a (C₁-C₆) alkyl group, a halo (C₁-C₆) alkyl group, a (C₁-C₆) alkoxy group, a halo (C₁-C₆) alkoxy group, a (C₁-C₆) alkylthio group, a halo (C₁-C₆) alkylthio group, a (C₁-C₆) alkylsulfinyl group, a halo (C₁-C₆) alkylsulfinyl group, a (C₁-C₆) alkylsulfonyl group and a halo (C₁-C₆) alkylsulfonyl group),
G⁵ represents C—R⁴ or N
(wherein
R⁴ represents:
(d1) a hydrogen atom;
(d2) a halogen atom;
(d3) a (C₁-C₆) alkyl group;
(d4) a (C₃-C₆) cycloalkyl group;
(d5) a (C₂-C₆) alkenyl group;
(d6) a (C₂-C₆) alkynyl group;
(d7) a (C₁-C₆) alkoxy group;
(d8) a halo (C₁-C₆) alkyl group;
(d9) a halo (C₃-C₆) cycloalkyl group;
(d10) a halo (C₂-C₆) alkenyl group;
(d11) a halo (C₂-C₆) alkynyl group; or
(d12) a halo (C₁-C₆) alkoxy group),
G⁶ represents N—R⁵, O or S
(wherein
R⁵ represents:
(e1) a hydrogen atom;
(e2) a (C₁-C₆) alkyl group;
(e3) a (C₃-C₆) cycloalkyl group;
(e4) a (C₂-C₆) alkenyl group; or
(e5) a (C₂-C₆) alkynyl group),
G⁷ and G⁸ may be the same or different and each represent C—H or N, and
m represents an integer of 0 to 2}, or
a salt thereof.

22. The condensed heterocyclic compound or the salt according to claim 21, wherein
R¹ represents (a1) a (C₁-C₆) alkyl group,
R² is:
(b8) a halo (C₁-C₆) alkyl group;
(b15) a halo (C₁-C₆) alkylthio group; or
(b17) a halo (C₁-C₆) alkylsulfonyl group,
G¹, G², G³ and G⁴ each represent C—R³
(wherein
R³s may be the same or different and represents:
(c1) a hydrogen atom;
(c2) a halogen atom;
(c4) a formyl group;
(c5) a cyano group;
(c6) a (C₁-C₆) alkyl group;
(c7) a (C₃-C₆) cycloalkyl group;
(c8) a (C₁-C₆) alkoxy group;
(c9) a halo (C₁-C₆) alkyl group;
(c12) a (C₁-C₆) alkylthio group;
(c15) a halo (C₁-C₆) alkylthio group;
(c19) an R⁹(R¹⁰)N group (wherein R⁹ and R¹⁰ may be the same or different and each represent a hydrogen atom; a (C₁-C₆) alkyl group; a (C₃-C₆) cycloalkyl (C₁-C₆) alkyl group; a halo (C₁-C₆) alkyl group; a (C₁-C₆) alkylcarbonyl group; a (C₁-C₆) alkoxycarbonyl group; or a phenylcarbonyl group);
(c22) a C(R⁹)═NOR¹⁰ group (wherein R⁹ and R¹⁰ are as defined above);
(c24) a (C₁-C₆) alkoxycarbonyl group; or
(c25) an R⁹(R¹⁰)N carbonyl group (wherein R⁹ and R¹⁰ are as defined above)),
G⁵ represents C—R⁴ (wherein R⁴ represents (d1) a hydrogen atom), and
G⁶ represents N—R⁵, O or S (wherein R⁵ represents (e2) a (C₁-C₆) alkyl group).

23. The condensed heterocyclic compound or the salt according to claim 21, wherein
R² is:
(b8) a halo (C₁-C₆) alkyl group or
(b15) a halo (C₁-C₆) alkylthio group,
G¹, G², G³ and G⁴ each represent C—R³
(wherein
R³s may be the same or different and represents:
(c1) a hydrogen atom;
(c2) a halogen atom; or
(c9) a halo (C₁-C₆) alkyl group), and
G⁶ represents N—R⁵ or O (wherein R⁵ represents (e2) a (C₁-C₆) alkyl group).

24. An agricultural and horticultural insecticide comprising the condensed heterocyclic compound or the salt according to claim 21 as an active ingredient.

25. A method for using an agricultural and horticultural insecticide, comprising treating plants or soil with an effective amount of the agricultural and horticultural insecticide according to claim 24.
